(19) United States
(12) Patent Application Publication
(10) Pub. No.: US 2024/0409566 A1
(43) Pub. Date: Dec. 12, 2024

(54) DELIVERY OF THERAPEUTIC ALKALOID COMPOUNDS

(71) Applicant: Sensorium Therapeutics, Inc., Boston, MA (US)

(72) Inventors: Michael S. Placzek, Tewksbury, MA (US); Simon N. Pettit, Colchester (GB)

(21) Appl. No.: 18/806,984

(22) Filed: Aug. 16, 2024

Related U.S. Application Data
(63) Continuation of application No. PCT/US23/85277, filed on Dec. 21, 2023.
(60) Provisional application No. 63/434,723, filed on Dec. 22, 2022.

(51) Int. Cl. C07F 9/572; A61K 31/404; A61K 31/675; C07D 209/12

(52) U.S. Cl. CPC C07F 9/5728; A61K 31/404; A61K 31/675; C07D 209/12

(57) ABSTRACT

Disclosed are compounds that can be converted to mesembrine under biologically relevant conditions, such as acid hydrolysis at body temperature; and related methods of preparing and using these compounds. Stable preparations of isolated mesembrine stereoisomers are also provided.

### RELATED APPLICATIONS

This application claims the benefit of priority to U.S. Provisional Patent Application Ser. No. 63/434,723, filed Dec. 22, 2022; the contents of which are hereby incorporated by reference in their entirety.

### TECHNICAL FIELD

The present disclosure relates to the field of medicine, including the discovery of alkaloid compounds useful for inhibiting the serotonin transporter protein (5-HTT).

### BACKGROUND

Plants of the genus Sceletium contain indole alkaloids having biological activity useful in treating mental health conditions such as mild to moderate depression. Natural extracts of Sceletium tortuosum, an indigenous herb of South Africa also referred to as “kougoed”, “channa” or “kanna,” can contain the pharmacologically active alkaloids. Mesembrine and mesembrenol, among the alkaloids shown below, are present in Sceletium tortuosum extracts used for treatment of anxiety, stress and mental health conditions. Mesembranol has lower concentrations in the extracts compared to the other alkaloids.

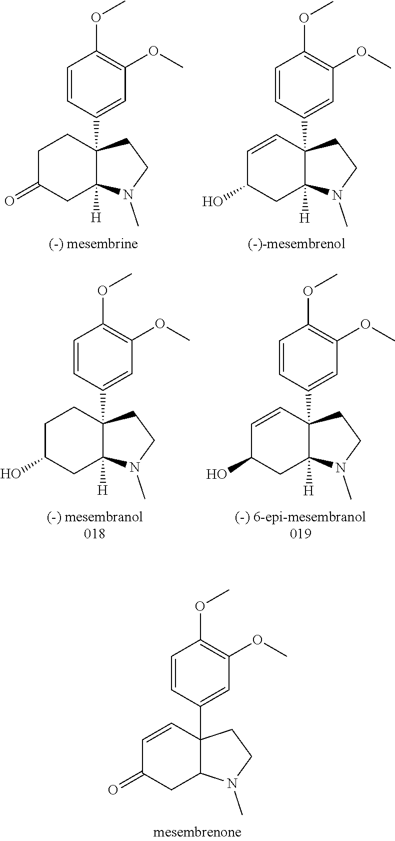 [SMILES: COc1ccc([C@@]23CCC(=O)C[C@@H]2N(C)CC3)cc1OC ; COc1ccc([C@@]23C=C[C@@H](O)C[C@@H]2N(C)CC3)cc1OC ; COc1ccc([C@@]23CC[C@@H](O)C[C@@H]2N(C)CC3)cc1OC ; COc1ccc([C@@]23C=C[C@H](O)C[C@@H]2N(C)CC3)cc1OC ; COc1ccc(C23C=CC(=O)CC2N(C)CC3)cc1OC]

An analysis of a standardized commercial extract of Sceletium tortuosum was reported in 2011 (obtained as a product under the tradename, Zembrin®) as having 0.35%-0.45% total alkaloids, with mesembrenone and mesembrenol comprising ≥60%, and mesembrine contributing <20% (See Harvey et al., “Pharmacological actions of the South African medicinal and functional food plant Sceletium tortuosum and its principal alkaloids,” Journal of Ethnopharmacology 137 (2011) 1124-11292011 and Murbach et. al., “A toxicological safety assessment of a standardized extract of Sceletium tortuosum (Zembrin®) in rats,” Food and Chemical Toxicology 74 (2014) 190-199). The extract gave >80% inhibition at serotonin (5-HT) transporter with potency of the isolated alkaloids at the 5-HT transporter reported as shown in Table A below (Harvey et al., 2011). Referring to the data in Table A, concentration-dependent inhibition was found, with mesembrine being the more active compound (i.e., 20 times more potent than mesembrenone and 87 times more active than mesembrenol) in the 5-HT transporter assay. A toxicological safety assessment of this standardized extract was subsequently reported in 2014 (Murbach et al., 2014).

Table A. Summary of analysis of the concentration response curves of alkaloids on binding to the 5-HT transporter (Harvey et al., 2011)

5-HT transporter (SERT)

| Compound | Ki (nM) | nH |
|---|---|---|
| Mesembrine | 1.4 | 1.0 |
| Mesembrenone | 27 | 1.0 |
| Mesembrenol | 62 | 1.1 |

However, bioactive plant extracts for therapeutic consumption can vary widely both seasonally and between different Sceletium tortuosum plants, and fail to provide a sufficiently reproducible and stable phytochemical profile of desired biologically active components. Plants of the genus Sceletium and extracts thereof can vary widely in terms of the total alkaloid content, as well as the chemistry and relative concentrations of individual Sceletium plant derived alkaloids. In addition, it has been reported that mesembranol concentrations in Sceletium tortuosum can vary across regions of South Africa, and are relatively low in most plant extracts that were tested. Lastly, Sceletium alkaloids may be unstable under a variety of conditions that can occur during extraction from plant material, as well as during storage and formulation of the extract.

In Sceletium tortuosum extract, mesembranol has low concentrations compared to the other major alkaloids, and 6-epi-mesembranol is only detected in trace amounts. The therapeutic use of mesembranol and 6-epi-mesembranol has been limited by the abundance, variability, and instability of these alkaloids in natural extract products, and the instability and pharmacokinetic profile of these compounds as obtained from natural products. Lastly, reported plasma concentrations of Sceletium tortuosum alkaloids in a rodent pharmacokinetic study were low. To improve oral bioavailability of Sceletium tortuosum alkaloids, there is a need for prodrugs of mesembranol and 6-epi-mesembranol.

### SUMMARY

Described herein are compounds of formula (I):

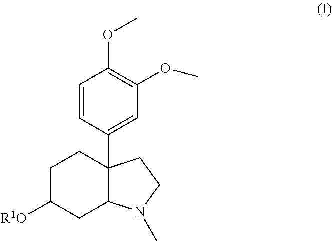

or a pharmaceutically acceptable salt thereof,
wherein:
R¹ is —C(O)NR²R³, —P(O)OR³OR⁴, —C(O)OR⁵, —C(O)R⁶, or —CH₂OC(O)R⁷;
R² is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy;
R³ is H, C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;
or R² and R³ taken together with the nitrogen atom to which they are attached form a 4-7 membered heterocycle, wherein each hydrogen atom in the 4-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;
R⁴ is H, C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;
or R³ and R⁴ taken together with the —O—P(O)—O— to which they are attached form a 5-7 membered heterocycle, wherein each hydrogen atom in the 5-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;
R⁵ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy;
R⁶ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, —OC(O)C₁-C₆ alkyl, nitro, —N(C₁-C₃ alkyl)₂, —NH₂, —N(H)C₁-C₃ alkyl, C₁-C₃ haloalkyl, —COOH, cyano, phenyl, or phenoxy;
R⁷ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy; and
n is 1 or 2.

In certain embodiments, the compound is of formula (I-A) or (I-B):

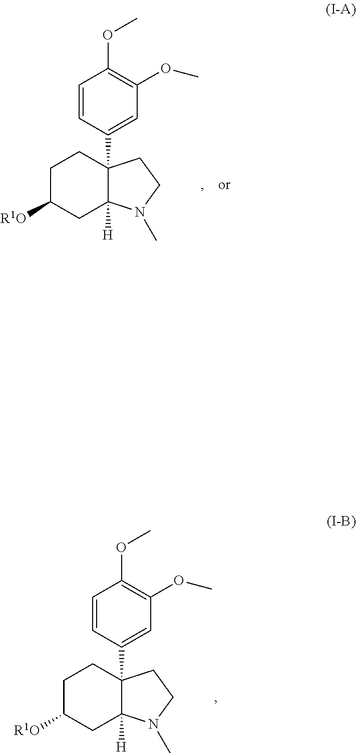

or a pharmaceutically acceptable salt thereof, wherein R¹ is as defined herein.

In certain embodiments, the compound is of formula (I-A) or (I-B):

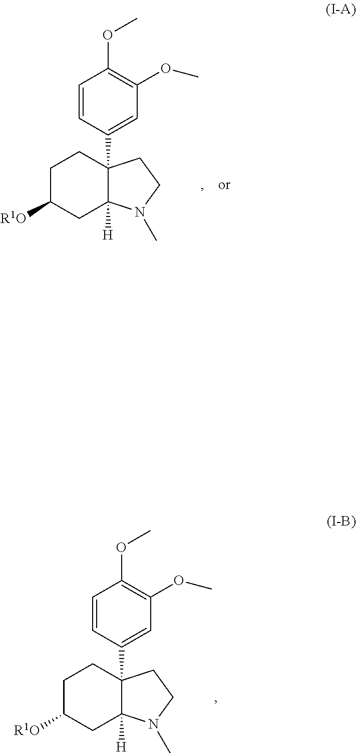

or a pharmaceutically acceptable salt thereof, wherein the compound has the absolute stereochemistry shown, and R¹ is as defined herein.

In certain embodiments, the compound is of formula (II):

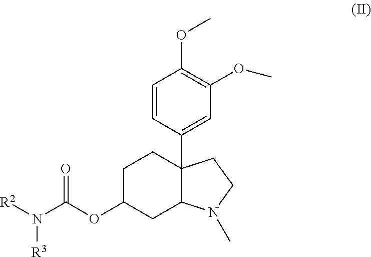

or a pharmaceutically acceptable salt thereof,
wherein:
R² is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy;
R³ is H, C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;
or R² and R³ taken together with the nitrogen atom to which they are attached form a 4-7 membered heterocycle, wherein each hydrogen atom in the 4-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy; and
n is 1 or 2.

In certain embodiments, the compound is of formula (II-A) or (II-B):

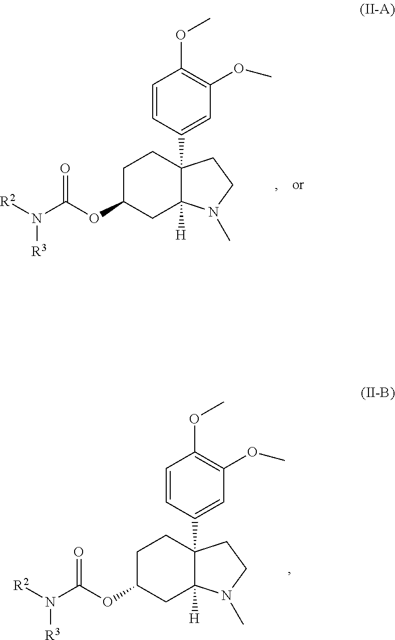

or a pharmaceutically acceptable salt thereof, wherein R² and R³ are as defined herein.

In certain embodiments, the compound is of formula (II-A) or (II-B):

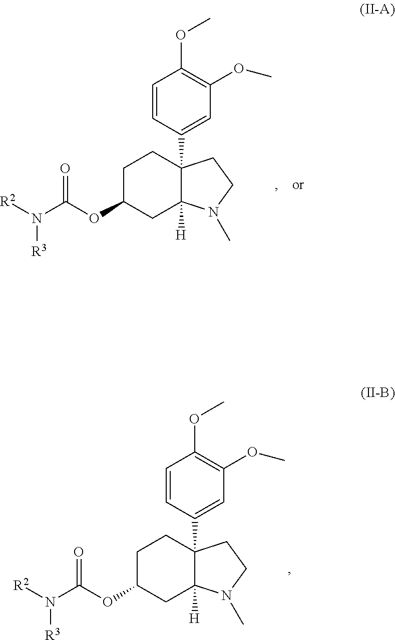

or a pharmaceutically acceptable salt thereof, wherein the compound has the absolute stereochemistry shown and R² and R³ are as defined herein.

In certain embodiments, the compound is of formula (III):

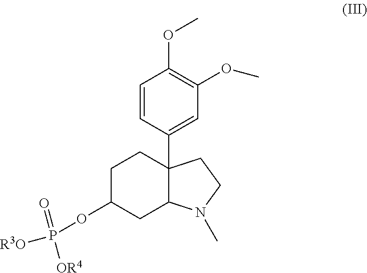

or a pharmaceutically acceptable salt thereof,
wherein:
R³ is H, C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by halo, hydroxy, C₁-C₃ heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;
R⁴ is H, C₁-C₆ alkyl, phenyl, —(CH₂O)ₙ—C(O)OC₁-C₆ alkyl, or —(CH₂O)ₙ—C(O) C₁-C₆ alkyl, wherein each hydrogen atom in C₁-C₆ alkyl is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;
or R³ and R⁴ taken together with the —O—P(O)—O— to which they are attached form a 5-7 membered heterocycle, wherein each hydrogen atom in the 5-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy; and
n is 1 or 2.

In certain embodiments, the compound is of formula (III-A) or (III-B):

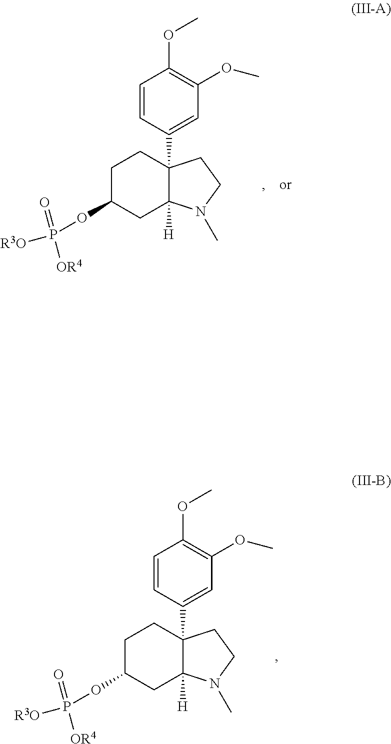

or a pharmaceutically acceptable salt thereof, wherein R³ and R⁴ are as defined herein.

In certain embodiments, the compound is of formula (III-A) or (III-B):

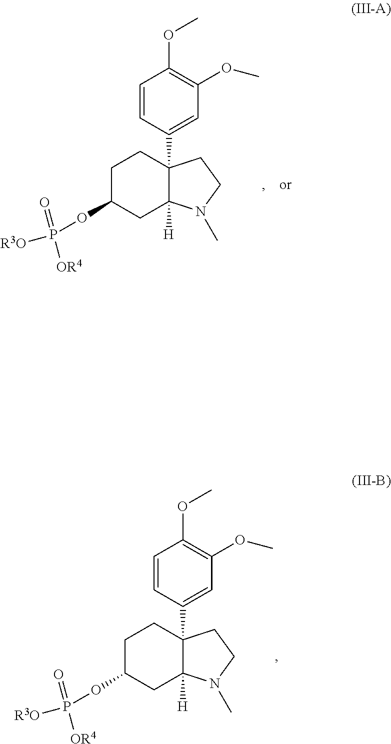

or a pharmaceutically acceptable salt thereof, wherein the compound has the absolute stereochemistry shown and R³ and R⁴ are as defined herein.

In certain embodiments, the compound is of formula (IV):

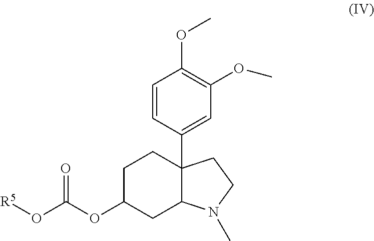

or a pharmaceutically acceptable salt thereof,
wherein:
R⁵ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy.

In certain embodiments, the compound is of formula (IV-A) or (IV-B):

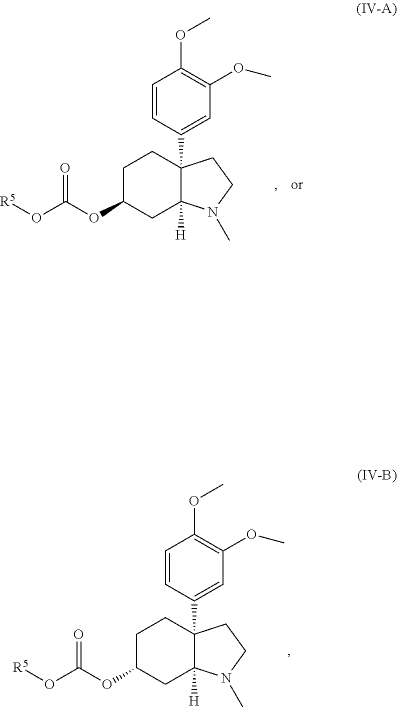

or a pharmaceutically acceptable salt thereof, wherein R⁵ is as defined herein.

In certain embodiments, the compound is of formula (IV-A) or (IV-B):

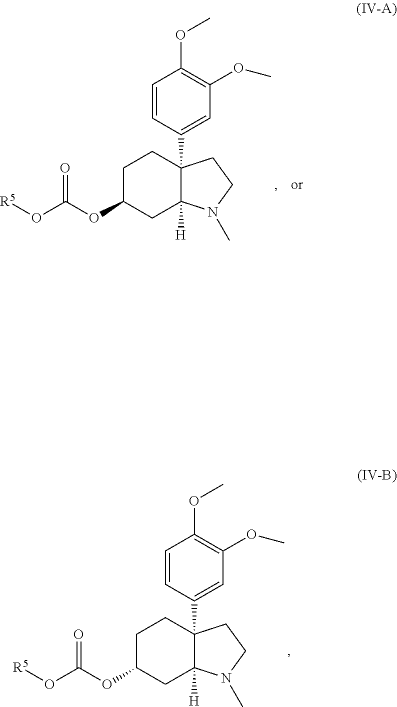

or a pharmaceutically acceptable salt thereof, wherein the compound has the absolute stereochemistry shown and R⁵ is as defined herein.

In certain embodiments, the compound is of formula (V):

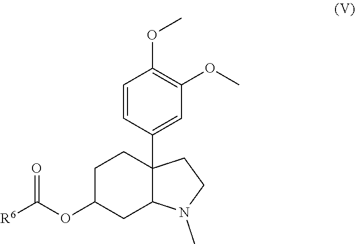

or a pharmaceutically acceptable salt thereof,
wherein:
R⁶ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, —OC(O)C₁-C₆ alkyl, nitro, —N(C₁-C₃ alkyl)₂, —NH₂, —N(H)C₁-C₃ alkyl, C₁-C₃ haloalkyl, —COOH, cyano, phenyl, or phenoxy.

In certain embodiments, the compound is of formula (V-A) or (V-B):

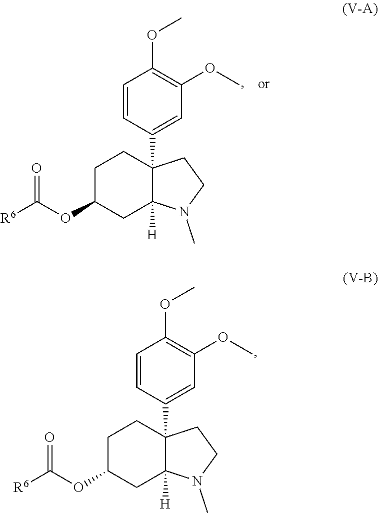

or a pharmaceutically acceptable salt thereof, wherein R⁶ is as defined herein.

In certain embodiments, the compound is of formula (V-A) or (V-B):

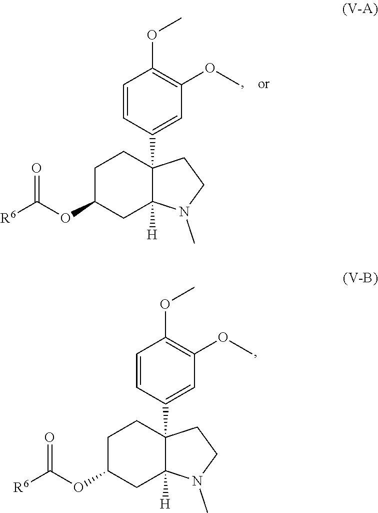

or a pharmaceutically acceptable salt thereof, wherein the compound has the absolute stereochemistry shown and R⁶ is as defined herein.

In certain embodiments, the compound is of formula (VI):

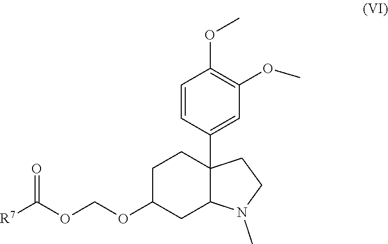

or a pharmaceutically acceptable salt thereof,
wherein:
R⁷ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy.

In certain embodiments, the compound is of formula (VI-A) or (VI-B):

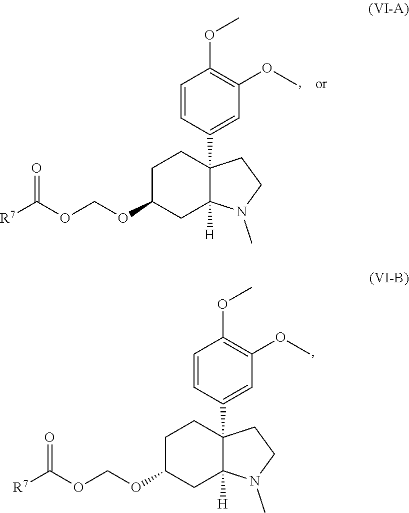

or a pharmaceutically acceptable salt thereof, wherein R⁷ is as defined herein.

In certain embodiments, the compound is of formula (VI-A) or (VI-B):

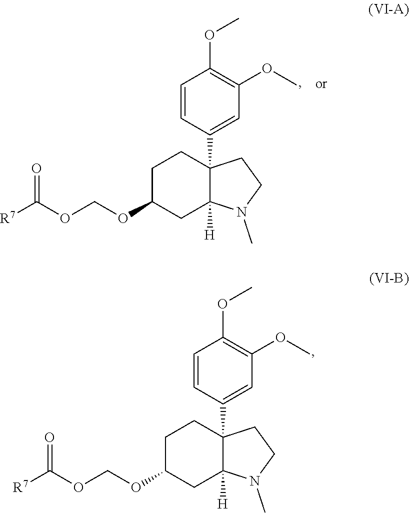

or a pharmaceutically acceptable salt thereof, wherein the compound has the absolute stercochemistry shown and R⁷ is as defined herein.

In certain embodiments, the compound is selected from:

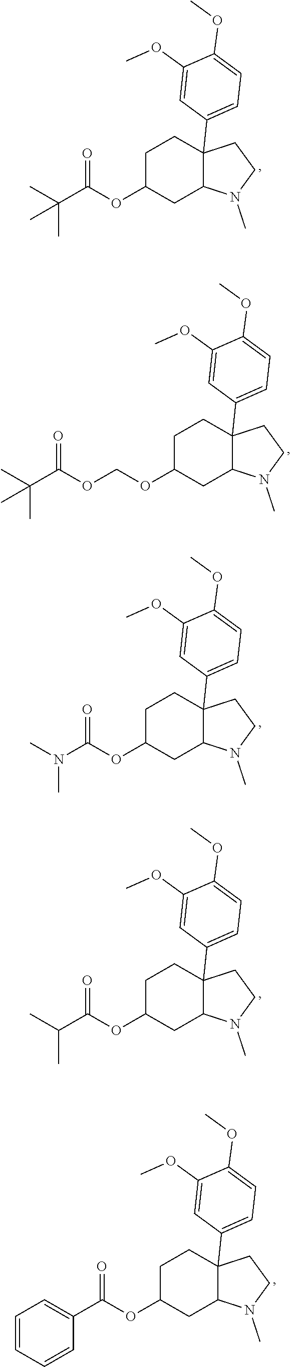 [SMILES: COc1ccc(C23CCC(OC(=O)C(C)(C)C)CC2N(C)CC3)cc1OC ; COc1ccc(C23CCC(OCOC(=O)C(C)(C)C)CC2N(C)CC3)cc1OC ; COc1ccc(C23CCC(OC(=O)N(C)C)CC2N(C)CC3)cc1OC ; COc1ccc(C23CCC(OC(=O)C(C)C)CC2N(C)CC3)cc1OC ; COc1ccc(C23CCC(OC(=O)c4ccccc4)CC2N(C)CC3)cc1OC]

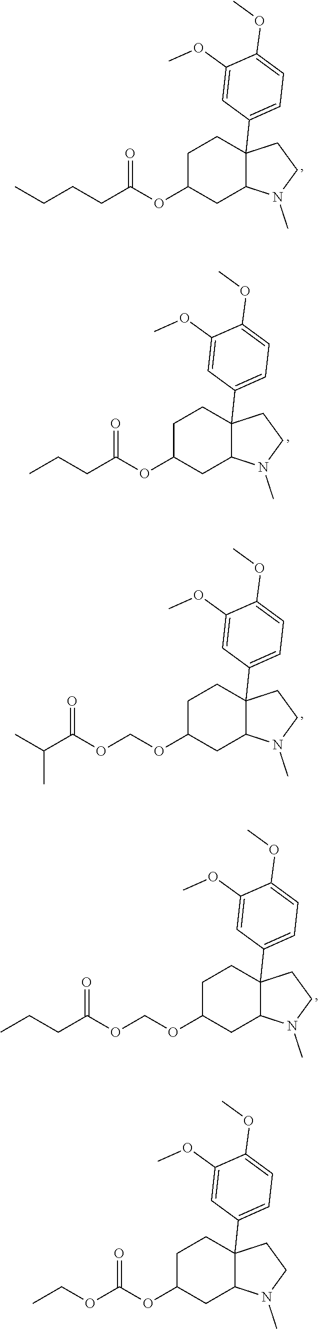 [SMILES: CCCCC(=O)OC1CCC2(c3ccc(OC)c(OC)c3)CCN(C)C2C1 ; CCCC(=O)OC1CCC2(c3ccc(OC)c(OC)c3)CCN(C)C2C1 ; COc1ccc(C23CCC(OCOC(=O)C(C)C)CC2N(C)CC3)cc1OC ; CCCC(=O)OCOC1CCC2(c3ccc(OC)c(OC)c3)CCN(C)C2C1 ; CCOC(=O)OC1CCC2(c3ccc(OC)c(OC)c3)CCN(C)C2C1]

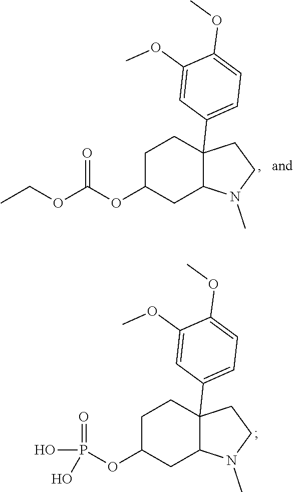 [SMILES: CCOC(=O)OC1CCC2(c3ccc(OC)c(OC)c3)CCN(C)C2C1 ; COc1ccc(C23CCC(OP(=O)(O)O)CC2N(C)CC3)cc1OC]

or a pharmaceutically acceptable salt thereof.

In certain embodiments, the compound is selected from:

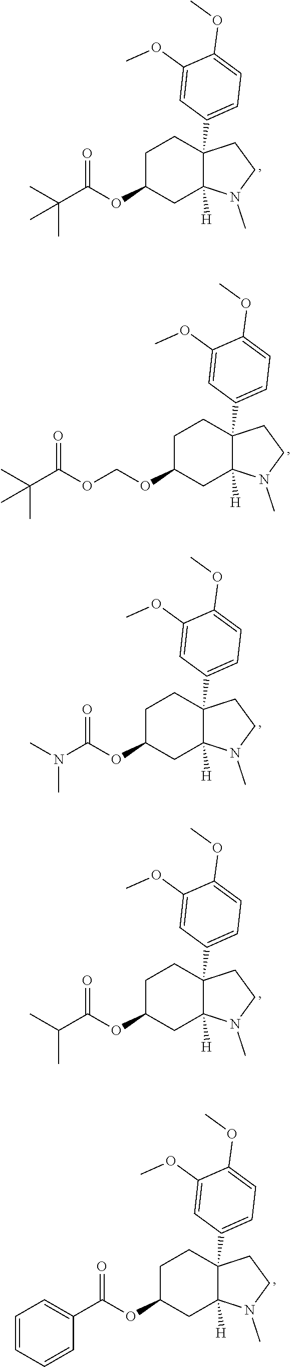 [SMILES: COc1ccc([C@@]23CC[C@H](OC(=O)C(C)(C)C)C[C@@H]2N(C)CC3)cc1OC ; COc1ccc([C@@]23CC[C@H](OCOC(=O)C(C)(C)C)C[C@@H]2N(C)CC3)cc1OC ; COc1ccc([C@@]23CC[C@H](OC(=O)N(C)C)C[C@@H]2N(C)CC3)cc1OC ; COc1ccc([C@@]23CC[C@H](OC(=O)C(C)C)C[C@@H]2N(C)CC3)cc1OC ; COc1ccc([C@@]23CC[C@H](OC(=O)c4ccccc4)C[C@@H]2N(C)CC3)cc1OC]

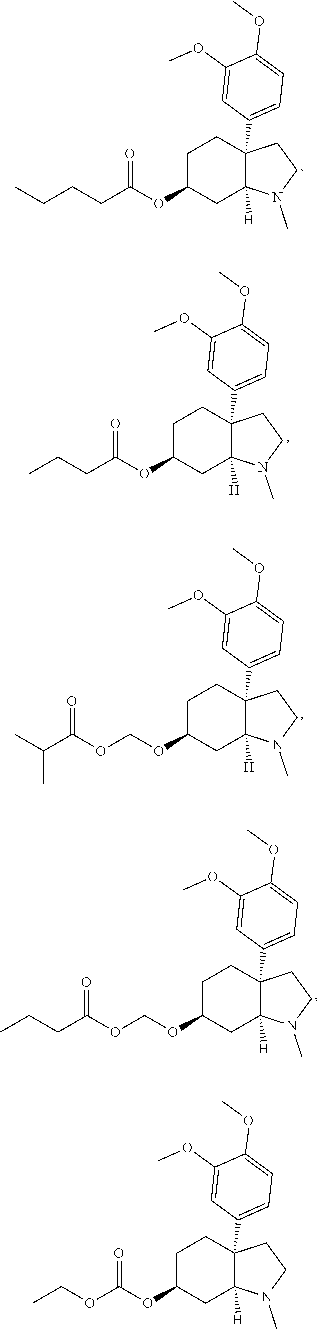 [SMILES: CCCCC(=O)O[C@H]1CC[C@@]2(c3ccc(OC)c(OC)c3)CCN(C)[C@H]2C1 ; CCCC(=O)O[C@H]1CC[C@@]2(c3ccc(OC)c(OC)c3)CCN(C)[C@H]2C1 ; COc1ccc([C@@]23CC[C@H](OCOC(=O)C(C)C)C[C@@H]2N(C)CC3)cc1OC ; CCCC(=O)OCO[C@H]1CC[C@@]2(c3ccc(OC)c(OC)c3)CCN(C)[C@H]2C1 ; CCOC(=O)O[C@H]1CC[C@@]2(c3ccc(OC)c(OC)c3)CCN(C)[C@H]2C1]

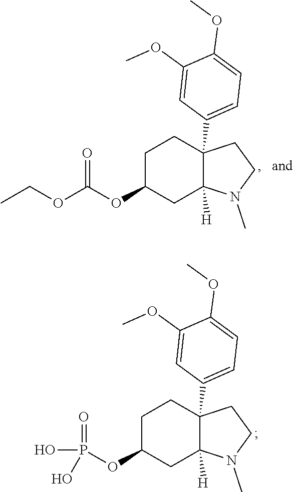 [SMILES: CCOC(=O)O[C@H]1CC[C@@]2(c3ccc(OC)c(OC)c3)CCN(C)[C@H]2C1 ; COc1ccc([C@@]23CC[C@H](OP(=O)(O)O)C[C@@H]2N(C)CC3)cc1OC]

or a pharmaceutically acceptable salt thereof.

In certain embodiments, the compound is selected from:

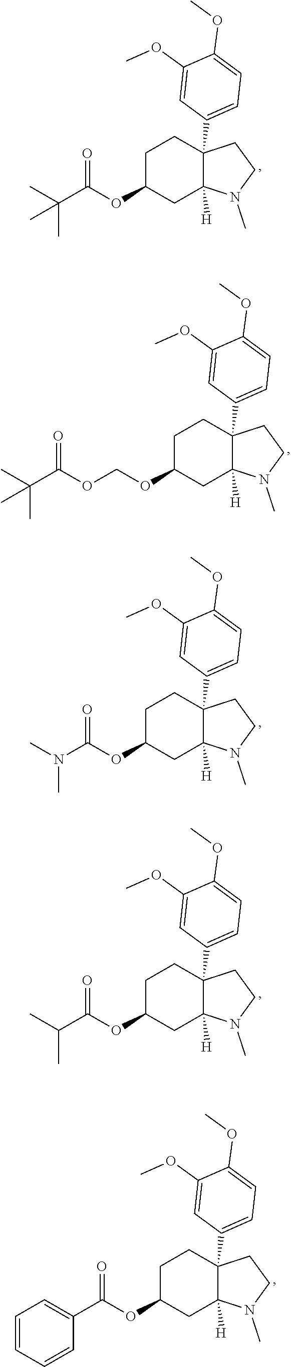 [SMILES: COc1ccc([C@@]23CC[C@H](OC(=O)C(C)(C)C)C[C@@H]2N(C)CC3)cc1OC ; COc1ccc([C@@]23CC[C@H](OCOC(=O)C(C)(C)C)C[C@@H]2N(C)CC3)cc1OC ; COc1ccc([C@@]23CC[C@H](OC(=O)N(C)C)C[C@@H]2N(C)CC3)cc1OC ; COc1ccc([C@@]23CC[C@H](OC(=O)C(C)C)C[C@@H]2N(C)CC3)cc1OC ; COc1ccc([C@@]23CC[C@H](OC(=O)c4ccccc4)C[C@@H]2N(C)CC3)cc1OC]

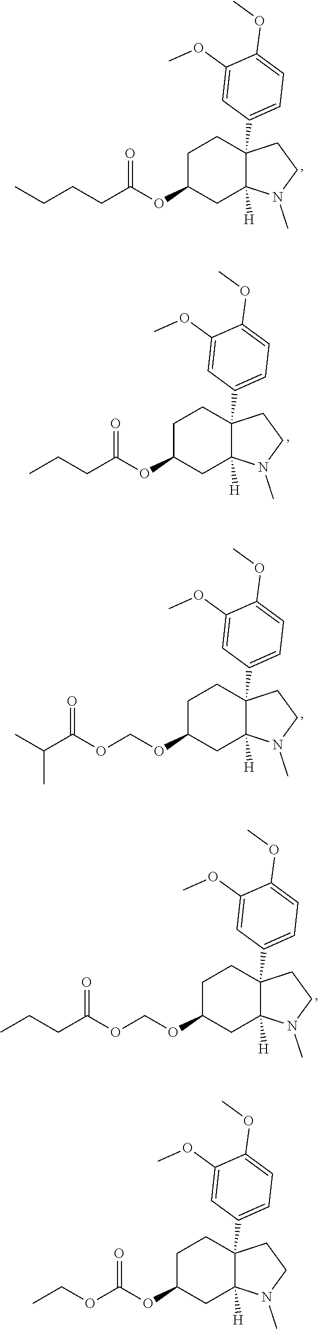 [SMILES: CCCCC(=O)O[C@H]1CC[C@@]2(c3ccc(OC)c(OC)c3)CCN(C)[C@H]2C1 ; CCCC(=O)O[C@H]1CC[C@@]2(c3ccc(OC)c(OC)c3)CCN(C)[C@H]2C1 ; COc1ccc([C@@]23CC[C@H](OCOC(=O)C(C)C)C[C@@H]2N(C)CC3)cc1OC ; CCCC(=O)OCO[C@H]1CC[C@@]2(c3ccc(OC)c(OC)c3)CCN(C)[C@H]2C1 ; CCOC(=O)O[C@H]1CC[C@@]2(c3ccc(OC)c(OC)c3)CCN(C)[C@H]2C1]

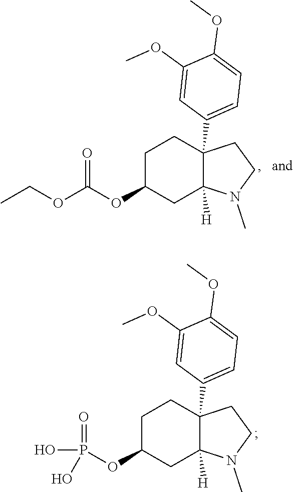 [SMILES: CCOC(=O)O[C@H]1CC[C@@]2(c3ccc(OC)c(OC)c3)CCN(C)[C@H]2C1 ; COc1ccc([C@@]23CC[C@H](OP(=O)(O)O)C[C@@H]2N(C)CC3)cc1OC]

or a pharmaceutically acceptable salt thereof; wherein the compound has the absolute stercochemistry shown.

In certain embodiments, the compound is selected from:

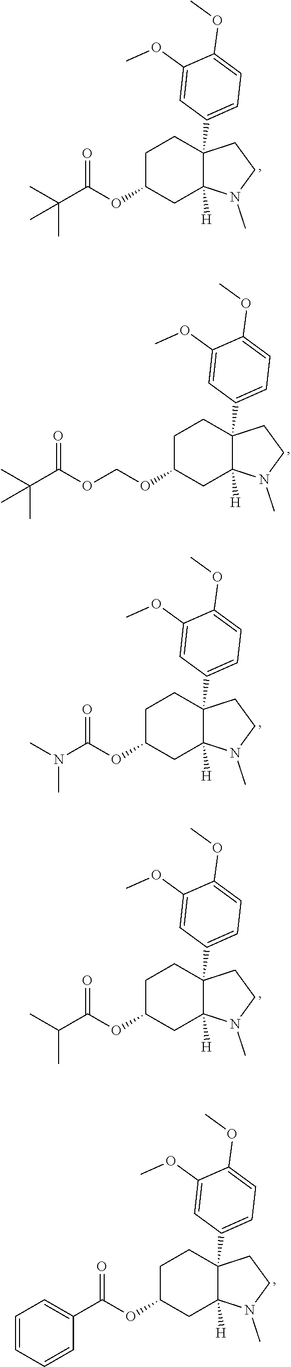 [SMILES: COc1ccc([C@@]23CC[C@@H](OC(=O)C(C)(C)C)C[C@@H]2N(C)CC3)cc1OC ; COc1ccc([C@@]23CC[C@@H](OCOC(=O)C(C)(C)C)C[C@@H]2N(C)CC3)cc1OC ; COc1ccc([C@@]23CC[C@@H](OC(=O)N(C)C)C[C@@H]2N(C)CC3)cc1OC ; COc1ccc([C@@]23CC[C@@H](OC(=O)C(C)C)C[C@@H]2N(C)CC3)cc1OC ; COc1ccc([C@@]23CC[C@@H](OC(=O)c4ccccc4)C[C@@H]2N(C)CC3)cc1OC]

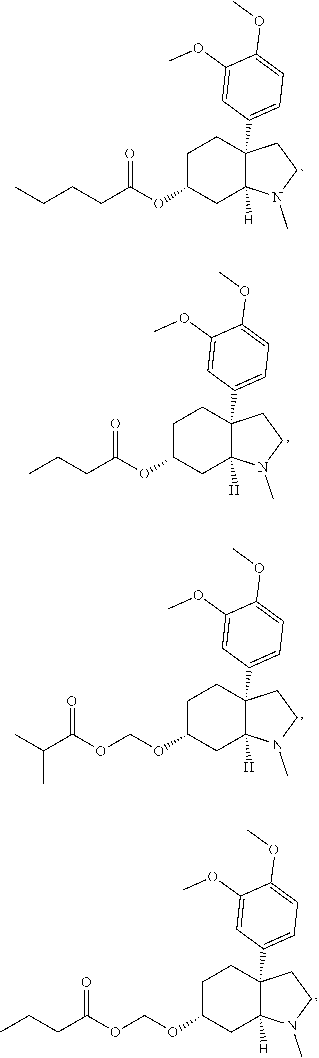 [SMILES: CCCCC(=O)O[C@@H]1CC[C@@]2(c3ccc(OC)c(OC)c3)CCN(C)[C@H]2C1 ; CCCC(=O)O[C@@H]1CC[C@@]2(c3ccc(OC)c(OC)c3)CCN(C)[C@H]2C1 ; COc1ccc([C@@]23CC[C@@H](OCOC(=O)C(C)C)C[C@@H]2N(C)CC3)cc1OC ; CCCC(=O)OCO[C@@H]1CC[C@@]2(c3ccc(OC)c(OC)c3)CCN(C)[C@H]2C1]

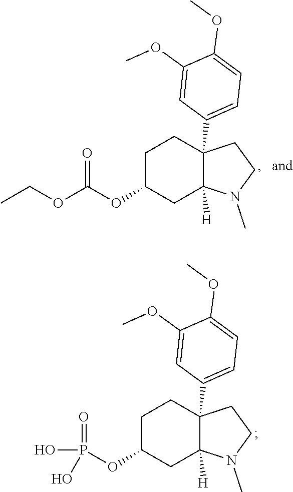 [SMILES: CCOC(=O)O[C@@H]1CC[C@@]2(c3ccc(OC)c(OC)c3)CCN(C)[C@H]2C1 ; COc1ccc([C@@]23CC[C@@H](OP(=O)(O)O)C[C@@H]2N(C)CC3)cc1OC]

or a pharmaceutically acceptable salt thereof.

In certain embodiments, the compound is selected from:

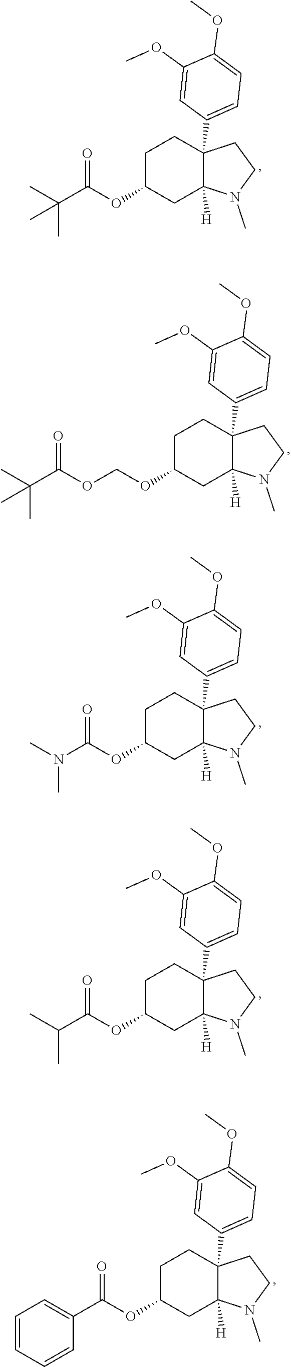 [SMILES: COc1ccc([C@@]23CC[C@@H](OC(=O)C(C)(C)C)C[C@@H]2N(C)CC3)cc1OC ; COc1ccc([C@@]23CC[C@@H](OCOC(=O)C(C)(C)C)C[C@@H]2N(C)CC3)cc1OC ; COc1ccc([C@@]23CC[C@@H](OC(=O)N(C)C)C[C@@H]2N(C)CC3)cc1OC ; COc1ccc([C@@]23CC[C@@H](OC(=O)C(C)C)C[C@@H]2N(C)CC3)cc1OC ; COc1ccc([C@@]23CC[C@@H](OC(=O)c4ccccc4)C[C@@H]2N(C)CC3)cc1OC]

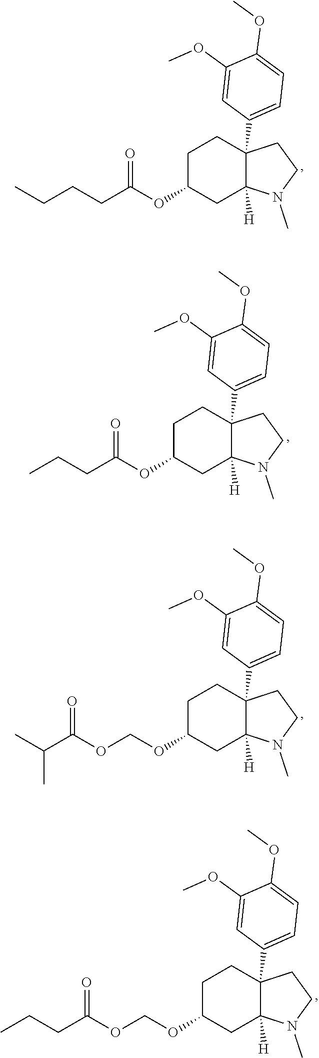 [SMILES: CCCCC(=O)O[C@@H]1CC[C@@]2(c3ccc(OC)c(OC)c3)CCN(C)[C@H]2C1 ; CCCC(=O)O[C@@H]1CC[C@@]2(c3ccc(OC)c(OC)c3)CCN(C)[C@H]2C1 ; COc1ccc([C@@]23CC[C@@H](OCOC(=O)C(C)C)C[C@@H]2N(C)CC3)cc1OC ; CCCC(=O)OCO[C@@H]1CC[C@@]2(c3ccc(OC)c(OC)c3)CCN(C)[C@H]2C1]

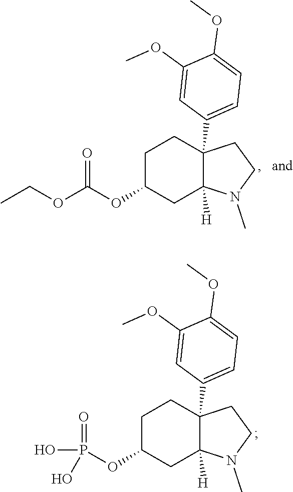 [SMILES: CCOC(=O)O[C@@H]1CC[C@@]2(c3ccc(OC)c(OC)c3)CCN(C)[C@H]2C1 ; COc1ccc([C@@]23CC[C@@H](OP(=O)(O)O)C[C@@H]2N(C)CC3)cc1OC]

or a pharmaceutically acceptable salt thereof; wherein the compound has the absolute stereochemistry shown.

In certain embodiments, the present disclosure provides a method of treating a mental disorder, comprising administering to the subject a compound of the present disclosure.

Numerous embodiments are further provided that can be applied to any aspect of the present invention described herein.

### DETAILED DESCRIPTION

It has been discovered that mesembranol is a potent inhibitor of serotonin transporter (SERT). Therefore, we sought to develop mesembranol compounds as oral therapeutics for mood disorders. Prodrugs of mesembranol, which release mesembranol in vivo with improved oral bioavailability and/or pharmacokinetics compared to the natural product, have been developed.

The present invention is based, at least in part, on mesembranol and 6-epi-mesembranol and prodrugs thereof. To take advantage of the desirable properties of mesembranol (e.g., (−) mesembranol or (−) 6-epi-mesembranol) and improve upon absorption, distribution, metabolism and excretion (ADME) that impact pharmacokinetics (PK), compounds and compositions have been developed and described here.

### EXEMPLARLY COMPOUNDS OF THE INVENTION

In certain embodiments, compounds described herein can form mesembranol (e.g., (−) mesembranol or (−) 6-epi-mesembranol) under biologically relevant conditions. For example, in some embodiments, compounds of disclosed herein (e.g., compounds of Formula (I)) can hydrolyze in highly acidic environments (e.g., pH of about 2 at room temperature or more comparably stringent conditions typically encountered within the alimentary canal of a mammal) and/or can undergo enzymatic hydrolysis (e.g., in the blood) at a rate that is advantageous for providing a desired bioabsorption (% F) following oral administration by a mammal and leading to a desired pharmacokinetic profile of mesembranol (e.g., (−) mesembranol or (−) 6-epi-mesembranol) to the mammal.

In some embodiments, a compound according to the present disclosure is of formula (I):

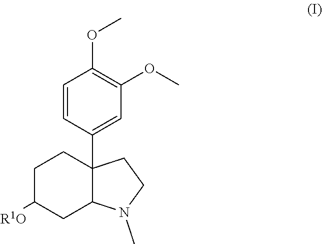

or a pharmaceutically acceptable salt thereof,
wherein:
R¹ is —C(O)NR²R³, —P(O)OR³OR⁴, —C(O)OR⁵, —C(O)R⁶, or —CH₂OC(O)R⁷;
R² is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy;
R³ is H, C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;
or R² and R³ taken together with the nitrogen atom to which they are attached form a 4-7 membered heterocycle, wherein each hydrogen atom in the 4-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;
R⁴ is C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;
or R³ and R⁴ taken together with the —O—P(O)—O— to which they are attached form a 5-7 membered heterocycle, wherein each hydrogen atom in the 5-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;
R⁵ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy;
R⁶ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, —OC(O)C₁-C₆ alkyl, nitro, —N(C₁-C₃ alkyl)₂, —NH₂, —N(H)C₁-C₃ alkyl, C₁-C₃ haloalkyl, —COOH, cyano, phenyl, or phenoxy;
R⁷ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy; and
n is 1 or 2.

In certain embodiments, the compound is of formula (I-A) or (I-B):

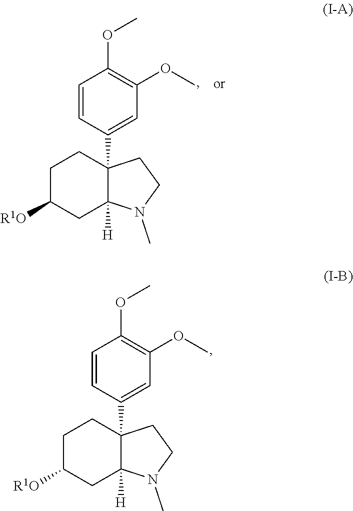

or a pharmaceutically acceptable salt thereof, wherein R¹ is as defined herein.

In certain embodiments, the compound is of formula (I-A) or (I-B):

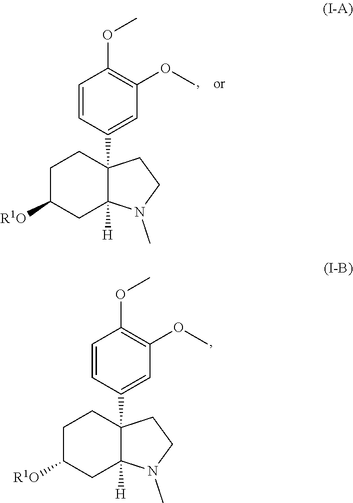

or a pharmaceutically acceptable salt thereof, wherein the compound has the absolute stereochemistry shown, and R¹ is as defined herein.

In certain embodiments, the compound is of formula (II):

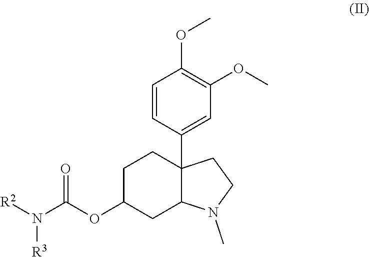

or a pharmaceutically acceptable salt thereof,
wherein:
R² is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy;
R³ is H, C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;
or R² and R³ taken together with the nitrogen atom to which they are attached form a 4-7 membered heterocycle, wherein each hydrogen atom in the 4-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy; and
n is 1 or 2.

In certain embodiments, the compound is of formula (II-A) or (II-B):

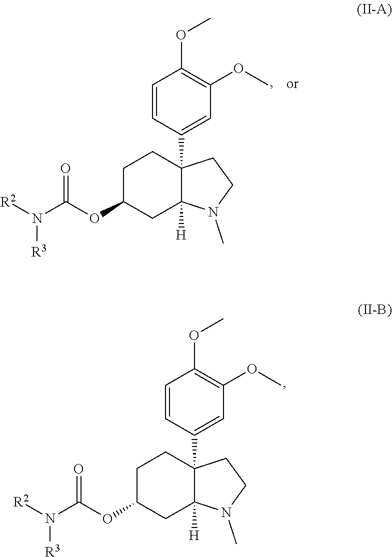

or a pharmaceutically acceptable salt thereof, wherein R² and R³ are as defined herein.

In certain embodiments, the compound is of formula (II-A) or (II-B):

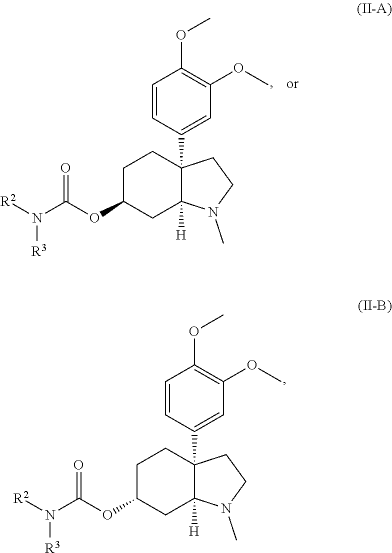

or a pharmaceutically acceptable salt thereof, wherein the compound has the absolute stereochemistry shown and R² and R³ are as defined herein.

In certain embodiments, the compound is of formula (III):

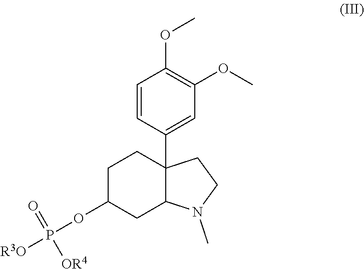

or a pharmaceutically acceptable salt thereof,
wherein:
R³ is H, C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by halo, hydroxy, C₁-C₃ heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;
R⁴ is C₁-C₆ alkyl, phenyl, —(CH₂O)ₙ—C(O)OC₁-C₆ alkyl, or —(CH₂O)ₙ—C(O) C₁-C₆ alkyl, wherein each hydrogen atom in C₁-C₆ alkyl is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;
or R³ and R⁴ taken together with the —O—P(O)—O— to which they are attached form a 5-7 membered heterocycle, wherein each hydrogen atom in the 5-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy; and
n is 1 or 2.

In certain embodiments, the compound is of formula (III-A) or (III-B):

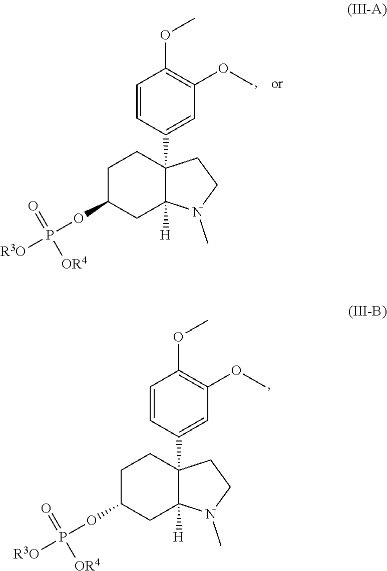

or a pharmaceutically acceptable salt thereof, wherein R³ and R⁴ are as defined herein.

In certain embodiments, the compound is of formula (III-A) or (III-B):

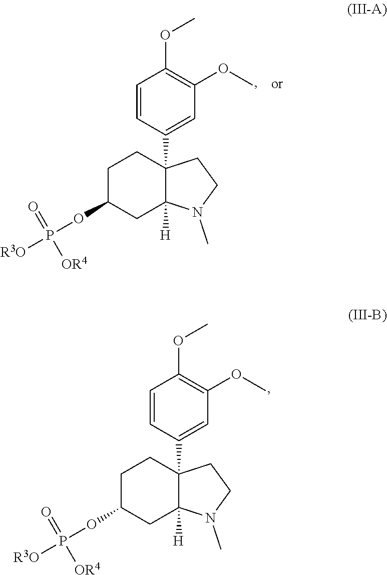

or a pharmaceutically acceptable salt thereof, wherein the compound has the absolute stereochemistry shown and R³ and R⁴ are as defined herein.

In certain embodiments, the compound is of formula (IV):

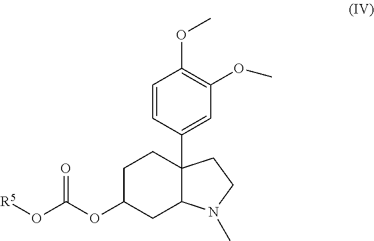

or a pharmaceutically acceptable salt thereof,
wherein:
R⁵ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy.

In certain embodiments, the compound is of formula (IV-A) or (IV-B):

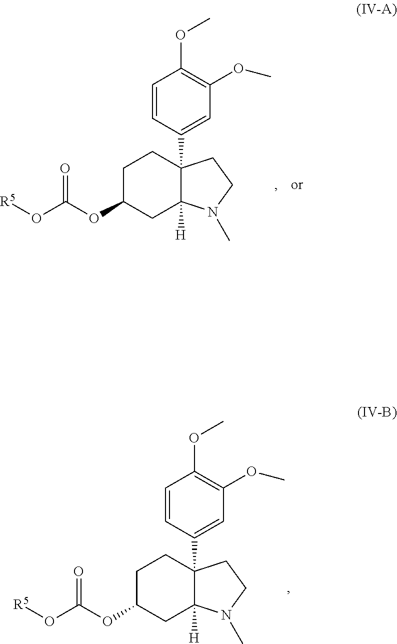

or a pharmaceutically acceptable salt thereof, wherein R⁵ is as defined herein.

In certain embodiments, the compound is of formula (IV-A) or (IV-B):

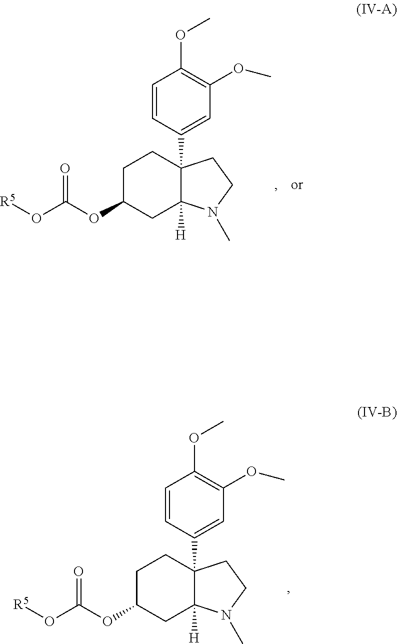

or a pharmaceutically acceptable salt thereof, wherein the compound has the absolute stereochemistry shown and R⁵ is as defined herein.

In certain embodiments, the compound is of formula (V):

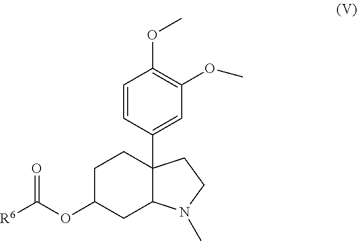

or a pharmaceutically acceptable salt thereof,
wherein:
R⁶ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, —OC(O)C₁-C₆ alkyl, nitro, —N(C₁-C₃ alkyl)₂, —NH₂, —N(H)C₁-C₃ alkyl, C₁-C₃ haloalkyl, —COOH, cyano, phenyl, or phenoxy.

In certain embodiments, the compound is of formula (V-A) or (V-B):

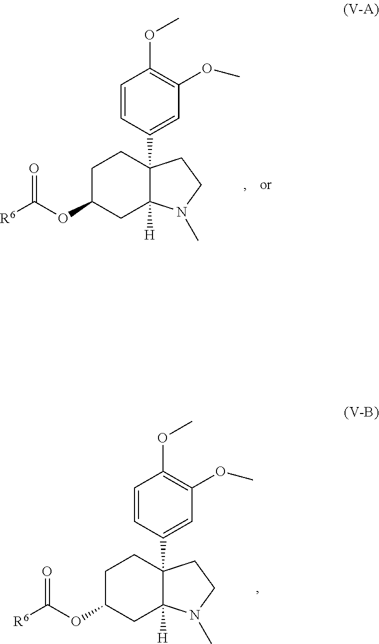

or a pharmaceutically acceptable salt thereof, wherein R⁶ is as defined herein.

In certain embodiments, the compound is of formula (V-A) or (V-B):

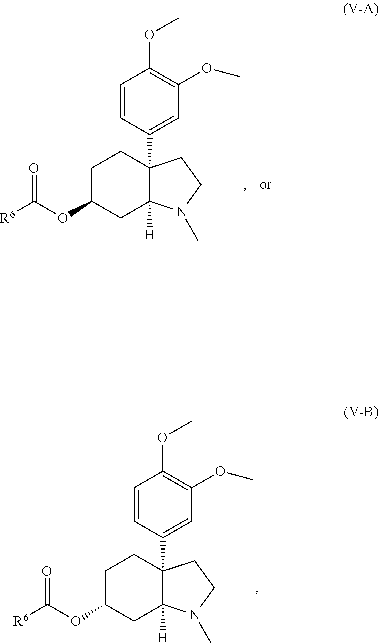

or a pharmaceutically acceptable salt thereof, wherein the compound has the absolute stereochemistry shown and R⁶ is as defined herein.

In certain embodiments, the compound is of formula (VI):

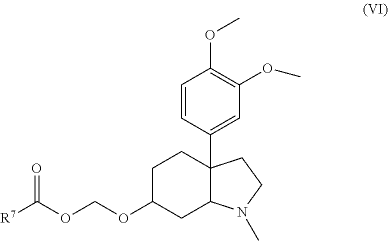

or a pharmaceutically acceptable salt thereof,
wherein:
R⁷ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy.

In certain embodiments, the compound is of formula (VI-A) or (VI-B):

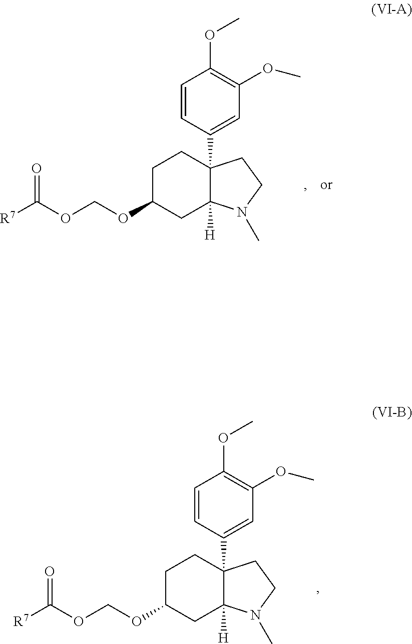

or a pharmaceutically acceptable salt thereof, wherein R⁷ is as defined herein.

In certain embodiments, the compound is of formula (VI-A) or (VI-B):

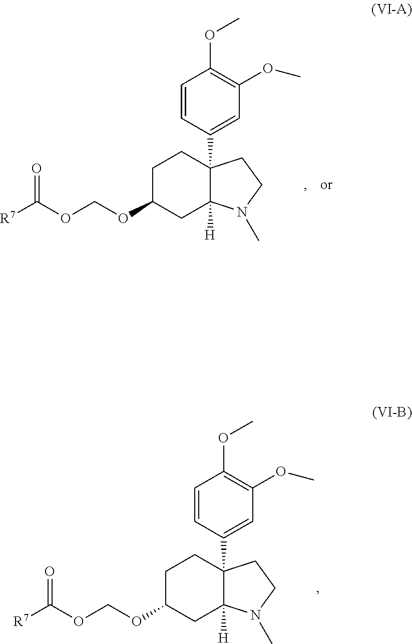

or a pharmaceutically acceptable salt thereof, wherein the compound has the absolute stercochemistry shown and R⁷ is as defined herein.

In certain embodiments, R¹ is —C(O)NR²R³. In some embodiments, R² is C₁-C₆ alkyl (e.g., methyl), C₂-C₆ alkenyl, C₃-C₁₀ cycloalkyl, C₁-C₃ alkyl-C₃-C₁₀ cycloalkyl, phenyl, or 5- to 7-membered heteroaryl. In some embodiments, R³ is C₁-C₆ alkyl (e.g., methyl), phenyl, —(CH₂O)ₙ—C(O)OC₁-C₆ alkyl, or —(CH₂O)ₙ—C(O)C₁-C₆ alkyl. In some embodiments, R³ is H. In some embodiments, each of R² and R³ is methyl. In some embodiments, R² and R³ are taken together with the nitrogen atom to which they are attached form a 4-7 membered heterocycle.

In certain embodiments, R¹ is —P(O)OR³OR⁴. In some embodiments, each of R³ and R⁴ is independently C₁-C₆ alkyl, phenyl, —(CH₂O)ₙ—C(O)OC₁-C₆ alkyl, or —(CH₂O)ₙ—C(O)C₁-C₆ alkyl. In some embodiments, R⁴ is C₁-C₆ alkyl, phenyl, —(CH₂O)ₙ—C(O)OC₁-C₆ alkyl, or —(CH₂O), —C(O)C₁-C₆ alkyl. In some embodiments, R³ is H. In some embodiments, n is 1 such that R¹ is —CH₂O—C(O)OC₁-C₆ alkyl. In some embodiments, R³ and R⁴ are taken together with the —O—P(O)—O— to which they are attached form a 5-7 membered heterocycle.

In certain embodiments, R¹ is —C(O)OR⁵. In some embodiments, R⁵ is C₁-C₆ alkyl (e.g., ethyl).

In certain embodiments, R¹ is —C(O)R⁶. In some embodiments, R⁶ is C₁-C₆ alkyl or phenyl. In some embodiments, R⁶ is C₁-C₆ alkyl (e.g., isopropyl, propyl, butyl, or t-butyl). In some embodiments, R⁶ is phenyl.

In certain embodiments, R¹ is —CH₂OC(O)R⁷. In some embodiments, R⁷ is C₁-C₆ alkyl (e.g., unsubstituted C₁-C₆ alkyl). In some embodiments, R⁷ is isopropyl, propyl, or t-butyl.

In certain embodiments, the compound is selected from:

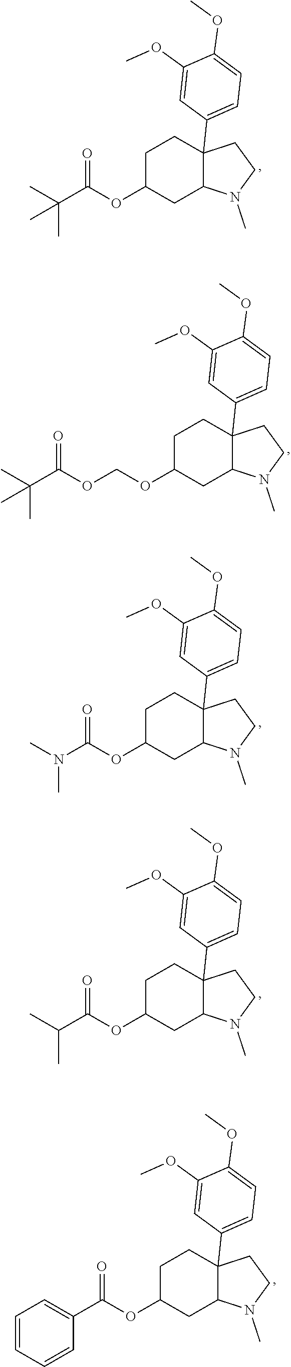 [SMILES: COc1ccc(C23CCC(OC(=O)C(C)(C)C)CC2N(C)CC3)cc1OC ; COc1ccc(C23CCC(OCOC(=O)C(C)(C)C)CC2N(C)CC3)cc1OC ; COc1ccc(C23CCC(OC(=O)N(C)C)CC2N(C)CC3)cc1OC ; COc1ccc(C23CCC(OC(=O)C(C)C)CC2N(C)CC3)cc1OC ; COc1ccc(C23CCC(OC(=O)c4ccccc4)CC2N(C)CC3)cc1OC]

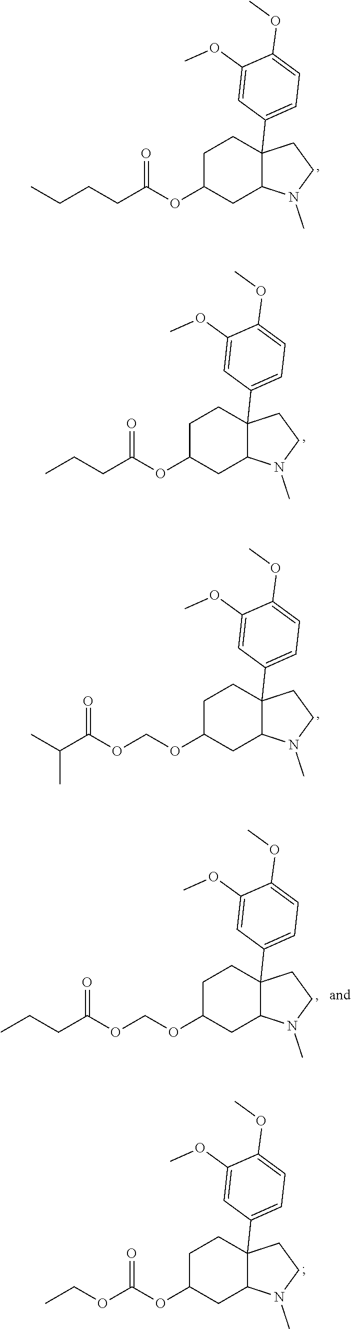 [SMILES: CCCCC(=O)OC1CCC2(c3ccc(OC)c(OC)c3)CCN(C)C2C1 ; CCCC(=O)OC1CCC2(c3ccc(OC)c(OC)c3)CCN(C)C2C1 ; COc1ccc(C23CCC(OCOC(=O)C(C)C)CC2N(C)CC3)cc1OC ; CCCC(=O)OCOC1CCC2(c3ccc(OC)c(OC)c3)CCN(C)C2C1 ; CCOC(=O)OC1CCC2(c3ccc(OC)c(OC)c3)CCN(C)C2C1]

or a pharmaceutically acceptable salt thereof.

In certain embodiments, the compound is selected from:

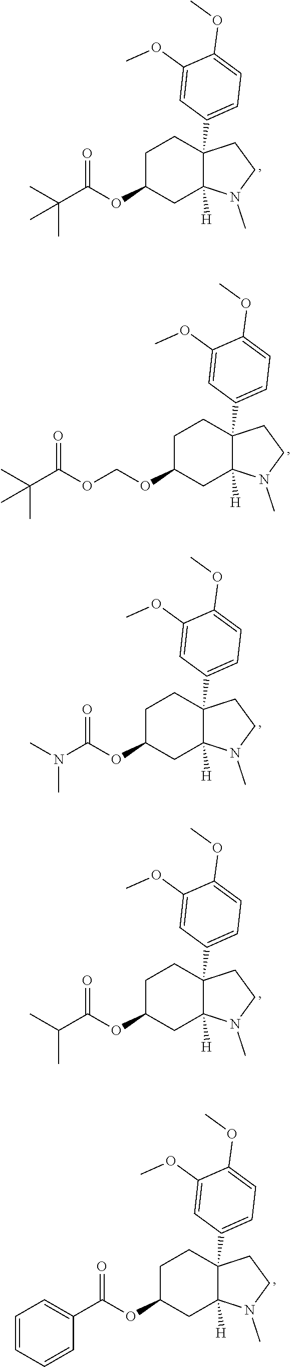 [SMILES: COc1ccc([C@@]23CC[C@H](OC(=O)C(C)(C)C)C[C@@H]2N(C)CC3)cc1OC ; COc1ccc([C@@]23CC[C@H](OCOC(=O)C(C)(C)C)C[C@@H]2N(C)CC3)cc1OC ; COc1ccc([C@@]23CC[C@H](OC(=O)N(C)C)C[C@@H]2N(C)CC3)cc1OC ; COc1ccc([C@@]23CC[C@H](OC(=O)C(C)C)C[C@@H]2N(C)CC3)cc1OC ; COc1ccc([C@@]23CC[C@H](OC(=O)c4ccccc4)C[C@@H]2N(C)CC3)cc1OC]

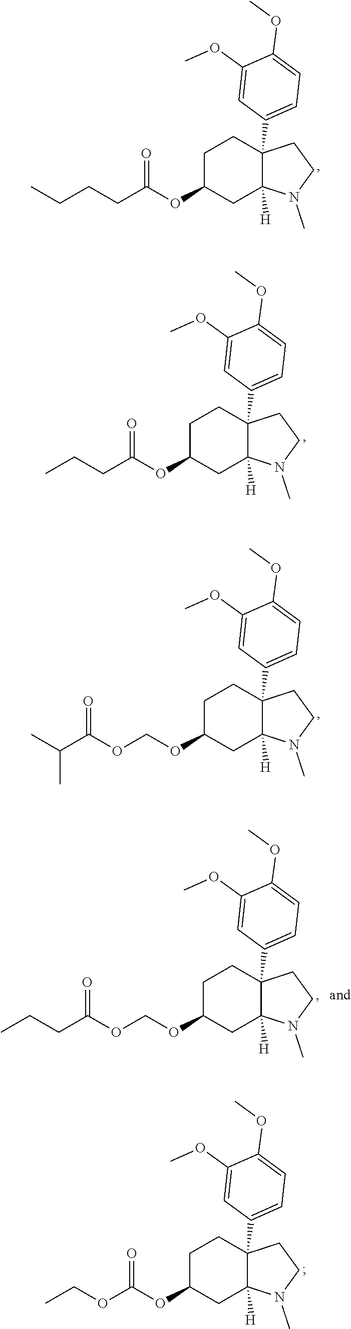 [SMILES: CCCCC(=O)O[C@H]1CC[C@@]2(c3ccc(OC)c(OC)c3)CCN(C)[C@H]2C1 ; CCCC(=O)O[C@H]1CC[C@@]2(c3ccc(OC)c(OC)c3)CCN(C)[C@H]2C1 ; COc1ccc([C@@]23CC[C@H](OCOC(=O)C(C)C)C[C@@H]2N(C)CC3)cc1OC ; CCCC(=O)OCO[C@H]1CC[C@@]2(c3ccc(OC)c(OC)c3)CCN(C)[C@H]2C1 ; CCOC(=O)O[C@H]1CC[C@@]2(c3ccc(OC)c(OC)c3)CCN(C)[C@H]2C1]

or a pharmaceutically acceptable salt thereof. In some embodiments, the compound has the absolute stercochemistry shown.

In some embodiments, the compound is selected from:

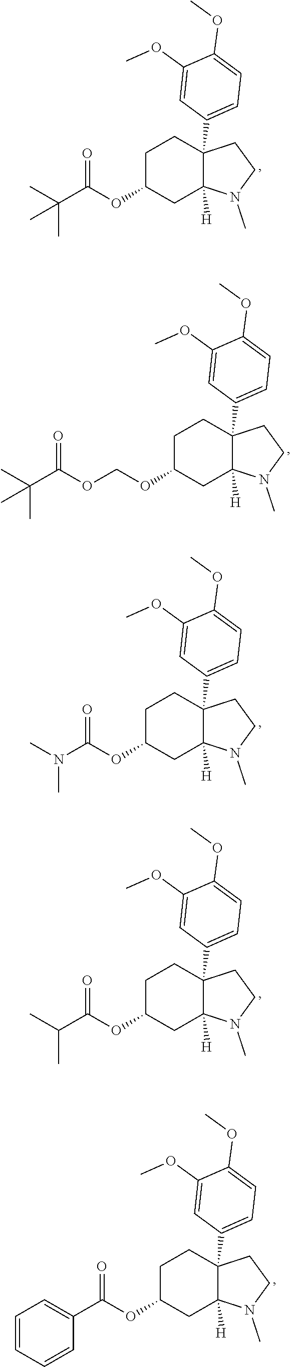 [SMILES: COc1ccc([C@@]23CC[C@@H](OC(=O)C(C)(C)C)C[C@@H]2N(C)CC3)cc1OC ; COc1ccc([C@@]23CC[C@@H](OCOC(=O)C(C)(C)C)C[C@@H]2N(C)CC3)cc1OC ; COc1ccc([C@@]23CC[C@@H](OC(=O)N(C)C)C[C@@H]2N(C)CC3)cc1OC ; COc1ccc([C@@]23CC[C@@H](OC(=O)C(C)C)C[C@@H]2N(C)CC3)cc1OC ; COc1ccc([C@@]23CC[C@@H](OC(=O)c4ccccc4)C[C@@H]2N(C)CC3)cc1OC]

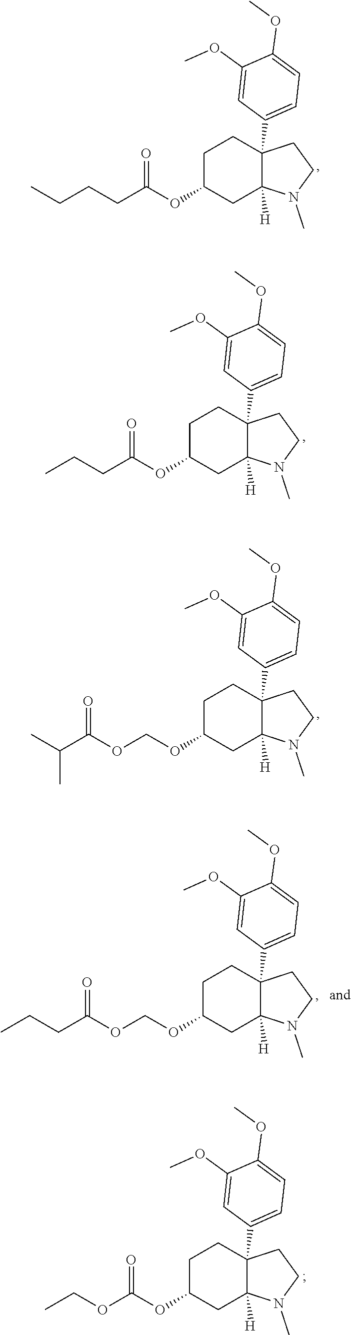 [SMILES: CCCCC(=O)O[C@@H]1CC[C@@]2(c3ccc(OC)c(OC)c3)CCN(C)[C@H]2C1 ; CCCC(=O)O[C@@H]1CC[C@@]2(c3ccc(OC)c(OC)c3)CCN(C)[C@H]2C1 ; COc1ccc([C@@]23CC[C@@H](OCOC(=O)C(C)C)C[C@@H]2N(C)CC3)cc1OC ; CCCC(=O)OCO[C@@H]1CC[C@@]2(c3ccc(OC)c(OC)c3)CCN(C)[C@H]2C1 ; CCOC(=O)O[C@@H]1CC[C@@]2(c3ccc(OC)c(OC)c3)CCN(C)[C@H]2C1]

or a pharmaceutically acceptable salt thereof. In some embodiments, the compound has the absolute stercochemistry shown.

In some embodiments, the compound is selected from

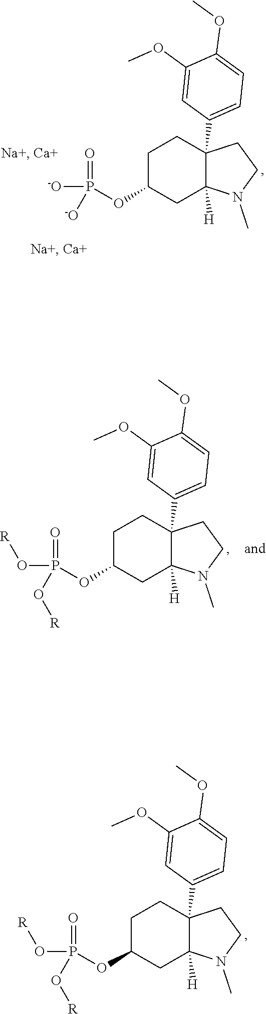

wherein each R is independently H, alkyl, aryl, heteroaryl, aralkyl, heteroaralkyl, acyl, —(CH₂O)ₙ—C(O)OC₁-C₆ alkyl, or —(CH₂O)ₙ—C(O)C₁-C₆ alkyl, wherein each hydrogen atom in alkyl, aryl, heteroaryl, aralkyl, heteroaralkyl is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy; or two Rs taken together with the —O—P(O)—O— to which they are attached form a 5-8 membered heterocycle, wherein each hydrogen atom in the 5-8 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy.

In certain embodiments, the compound is of formula (I-A) or (I-B):

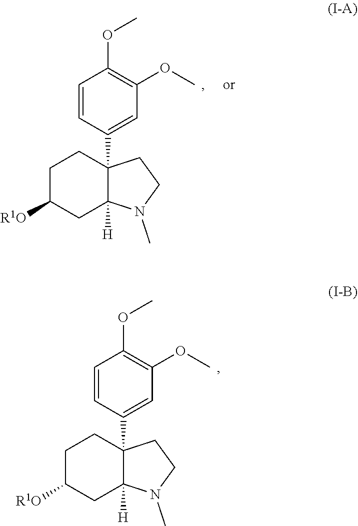

or a pharmaceutically acceptable salt thereof, wherein R¹ is —C(O)NR²R³; R² is C₁-C₆ alkyl; and R³ is H or C₁-C₆ alkyl. In certain embodiments, the compound is of formula (I-A) or (I-B), or a pharmaceutically acceptable salt thereof, wherein R¹ is —C(O)NR²R³; R² is C₁-C₆ alkyl; and R³ is C₁-C₆ alkyl. In certain embodiments, the compound is of formula (I-A) or (I-B), or a pharmaceutically acceptable salt thereof, wherein R¹ is —C(O)NR²R³; R² is C₁-C₄ alkyl; and R³ is C₁-C₄ alkyl. In certain embodiments, the compound is of formula (I-A) or (I-B), or a pharmaceutically acceptable salt thereof, wherein R¹ is —C(O)NR²R³; R² is methyl; and R³ is methyl.

In certain embodiments, the compound is of formula (I-A) or (I-B):

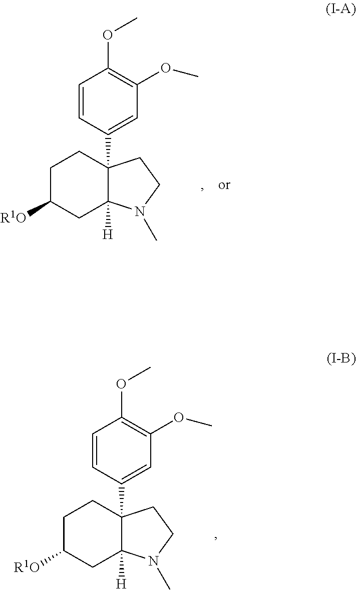

or a pharmaceutically acceptable salt thereof, wherein R¹ is —P(O)OR³OR⁴; R³ is H or C₁-C₆ alkyl, and R⁴ is H or C₁-C₆ alkyl. In certain embodiments, the compound is of formula (I-A) or (I-B), or a pharmaceutically acceptable salt thereof, wherein R¹ is —P(O)OR³OR⁴; R³ is H or C₁-C₄ alkyl, and R⁴ is H or C₁-C₄ alkyl. In certain embodiments, the compound is of formula (I-A) or (I-B), or a pharmaceutically acceptable salt thereof, wherein R¹ is —P(O)OR³OR⁴; R³ is H or methyl, and R⁴ is H or methyl. In certain embodiments, the compound is of formula (I-A) or (I-B), or a pharmaceutically acceptable salt thereof, wherein R¹ is —P(O)OR³OR⁴; R³ is H, and R⁴ is H.

In certain embodiments, the compound is of formula (I-A) or (I-B):

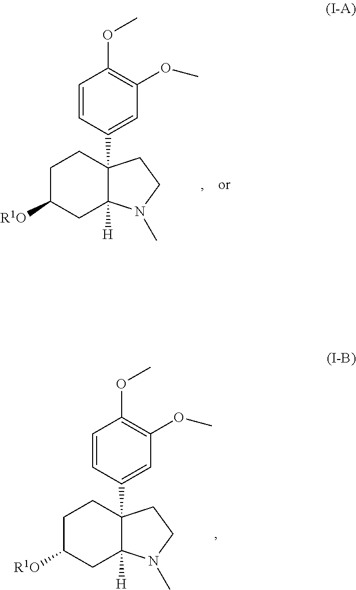

or a pharmaceutically acceptable salt thereof, wherein R¹ is —C(O)OR⁵; and R⁵ is C₁-C₆ alkyl. In certain embodiments, the compound is of formula (I-A) or (I-B), or a pharmaceutically acceptable salt thereof, wherein R¹ is —C(O)OR⁵; and R⁵ is C₁-C₄ alkyl. In certain embodiments, the compound is of formula (I-A) or (I-B), or a pharmaceutically acceptable salt thereof, wherein R¹ is —C(O)OR⁵; and R⁵ is C₂-C₄ alkyl. In certain embodiments, the compound is of formula (I-A) or (I-B), or a pharmaceutically acceptable salt thereof, wherein R¹ is —C(O)OR⁵, and R⁵ is methyl. In certain embodiments, the compound is of formula (I-A) or (I-B), or a pharmaceutically acceptable salt thereof, wherein R¹ is —C(O)OR⁵; and R⁵ is ethyl.

In certain embodiments, the compound is of formula (I-A) or (I-B):

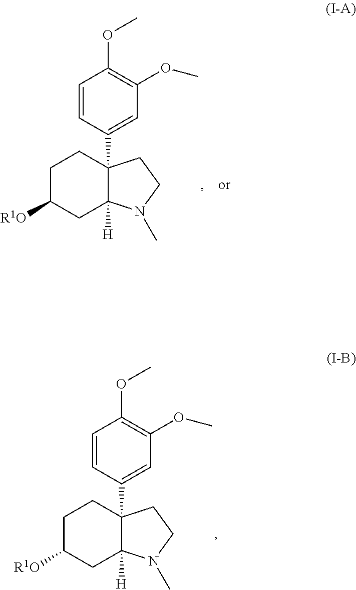

or a pharmaceutically acceptable salt thereof, wherein R¹ is —C(O)R⁶; and R⁶ is C₁-C₆ alkyl. In certain embodiments, the compound is of formula (I-A) or (I-B), or a pharmaceutically acceptable salt thereof, wherein R¹ is —C(O)R⁶; and R⁶ is n-butyl, isobutyl, or t-butyl. In certain embodiments, the compound is of formula (I-A) or (I-B), or a pharmaceutically acceptable salt thereof, wherein R¹ is —C(O)R⁶; and R⁶ is propyl. In certain embodiments, the compound is of formula (I-A) or (I-B), or a pharmaceutically acceptable salt thereof, wherein R¹ is —C(O)R⁶; and R⁶ is aryl optionally substituted by halo. In certain embodiments, the compound is of formula (I-A) or (I-B), or a pharmaceutically acceptable salt thereof, wherein R¹ is —C(O)R⁶; and R⁶ is phenyl optionally substituted by halo. In certain embodiments, the compound is of formula (I-A) or (I-B), or a pharmaceutically acceptable salt thereof, wherein R¹ is —C(O)R⁶; and R⁶ is 5-membered heteroaryl optionally substituted by halo. In certain embodiments, the compound is of formula (I-A) or (I-B), or a pharmaceutically acceptable salt thereof, wherein R¹ is —C(O)R⁶; and R⁶ is 6-membered heteroaryl optionally substituted by halo. In certain embodiments, the compound is of formula (I-A) or (I-B), or a pharmaceutically acceptable salt thereof, wherein R¹ is —C(O)R⁶; and R⁶ is pyridinyl. In certain embodiments, the compound is of formula (I-A) or (I-B), or a pharmaceutically acceptable salt thereof, wherein R¹ is —C(O)R⁶; and R⁶ is pyrimidinyl.

In certain embodiments, the compound is of formula (I-A) or (I-B):

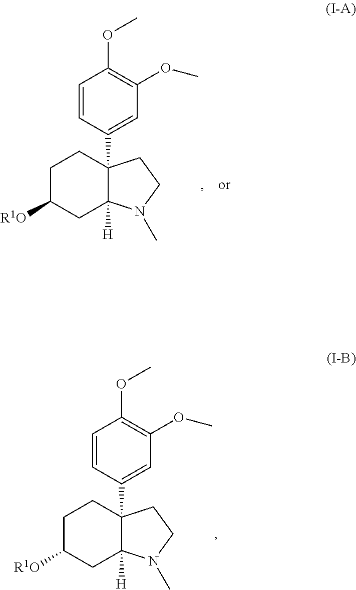

or a pharmaceutically acceptable salt thereof, wherein R¹ is —CH₂OC(O)R⁷; and R⁷ is C₁-C₆ alkyl. In certain embodiments, the compound is of formula (I-A) or (I-B), or a pharmaceutically acceptable salt thereof, wherein R¹ is —CH₂OC(O)R⁷; and R⁷ is propyl or butyl. In certain embodiments, the compound is of formula (I-A) or (I-B), or a pharmaceutically acceptable salt thereof, wherein R¹ is —CH₂OC(O)R⁷; and R⁷ is t-butyl, isobutyl, n-butyl, n-propyl, isopropyl, ethyl, or methyl.

In certain embodiments, the compound is of formula (II-A) or (II-B):

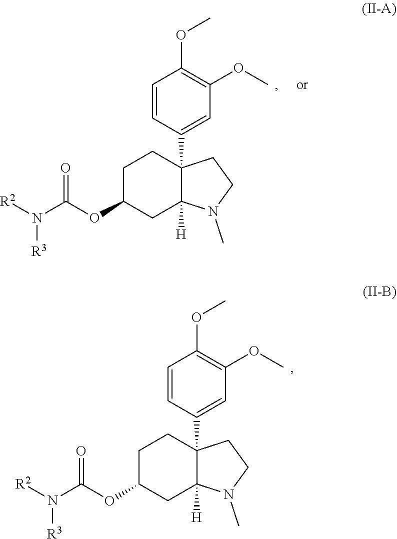

or a pharmaceutically acceptable salt thereof, wherein R² is C₁-C₆ alkyl and R³ is H or C₁-C₆ alkyl. In certain embodiments, the compound is of formula (II-A) or (II-B), or a pharmaceutically acceptable salt thereof, wherein R² is methyl and R³ is H or methyl. In certain embodiments, the compound is of formula (II-A) or (II-B), or a pharmaceutically acceptable salt thereof, wherein R² is methyl and R³ is methyl.

In certain embodiments, the compound is a compound of formula (III-A) or (III-B):

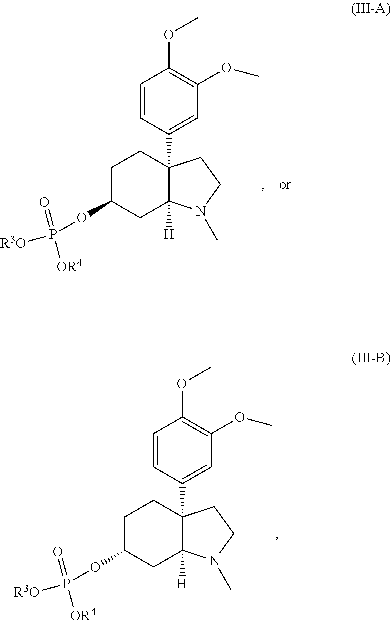

or a pharmaceutically acceptable salt thereof, wherein R³ is H or methyl, and R⁴ is H or methyl. In certain embodiments, the compound is of formula (III-A) or (III-B), or a pharmaceutically acceptable salt thereof, wherein R³ is H, and R⁴ is H or methyl. In certain embodiments, the compound is of formula (III-A) or (III-B), or a pharmaceutically acceptable salt thereof, wherein R³ is H or methyl, and R⁴ is H. In certain embodiments, the compound is of formula (III-A) or (III-B), or a pharmaceutically acceptable salt thereof, wherein R³ is methyl and R⁴ is H. In certain embodiments, the compound is of formula (III-A) or (III-B), or a pharmaceutically acceptable salt thereof, wherein R³ is H, and R⁴ is methyl. In certain embodiments, the compound is of formula (III-A) or (III-B), or a pharmaceutically acceptable salt thereof, wherein R³ is H and R⁴ is H.

In certain embodiments, the compound is of formula (IV-A) or (IV-B):

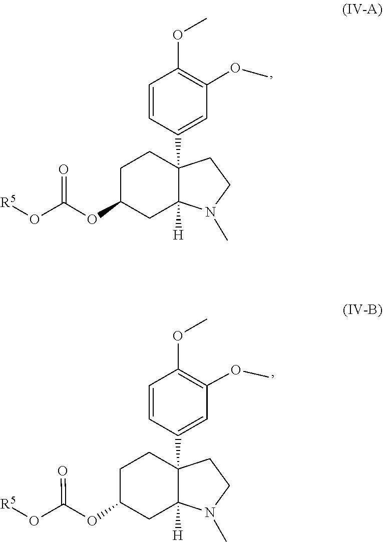

or a pharmaceutically acceptable salt thereof, wherein R⁵ is C₁-C₆ alkyl. In certain embodiments, the compound is of formula (IV-A) or (IV-B), or a pharmaceutically acceptable salt thereof, wherein R⁵ is ethyl.

In certain embodiments, the compound is of formula (V-A) or (V-B):

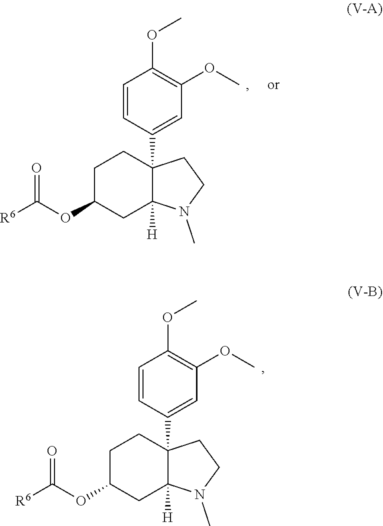

or a pharmaceutically acceptable salt thereof, wherein R⁶ is C₁-C₆ alkyl. In certain embodiments, the compound is of formula (V-A) or (V-B), or a pharmaceutically acceptable salt thereof, wherein R⁶ is C₃-C₆ alkyl. In certain embodiments, the compound is of formula (V-A) or (V-B), or a pharmaceutically acceptable salt thereof, wherein R⁶ is C₄-C₆ alkyl. In certain embodiments, the compound is of formula (V-A) or (V-B), or a pharmaceutically acceptable salt thereof, wherein R⁶ is t-butyl, isobutyl, or n-butyl. In certain embodiments, the compound is of formula (V-A) or (V-B), or a pharmaceutically acceptable salt thereof, wherein R⁶ is phenyl. In certain embodiments, the compound is of formula (V-A) or (V-B), or a pharmaceutically acceptable salt thereof, wherein R⁶ is pyridinyl. In certain embodiments, the compound is of formula (V-A) or (V-B), or a pharmaceutically acceptable salt thereof, wherein R⁶ is pyrimidinyl.

In certain embodiments, the compound is of formula (VI-A) or (VI-B):

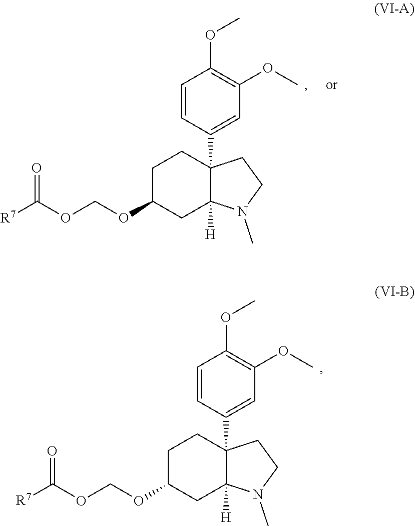

or a pharmaceutically acceptable salt thereof, wherein R⁷ is C₁-C₆ alkyl. In certain embodiments, the compound is of formula (VI-A) or (VI-B), or a pharmaceutically acceptable salt thereof, wherein R⁷ is C₃-C₆ alkyl. In certain embodiments, the compound is of formula (VI-A) or (VI-B), or a pharmaceutically acceptable salt thereof, wherein R⁷ is C₃-C₄ alkyl. In certain embodiments, the compound is of formula (VI-A) or (VI-B), or a pharmaceutically acceptable salt thereof, wherein R⁷ is butyl or propyl.

In some embodiments, methods of treating a patient suffering from a disease comprise administering to a patient a composition comprising a compound disclosed herein for the treatment or prevention of a mental health disorder. In some embodiments, methods of treating a patient suffering from a disease comprise administering to a patient a composition comprising a compound disclosed herein for the treatment or prevention of a diagnosed condition selected from anxiety and depression. In some embodiments, a method comprises the administration to a patient in need thereof of a therapeutically effective amount of a compound of Formula (I)-(VI) for the treatment of depression. In some embodiments, a method comprises the administration to a patient in need thereof of a therapeutically effective amount of a compound of Formula (I)-(VI) for the treatment of a condition selected from the group consisting of: anxiety associated with depression, anxiety with depression, mixed anxiety and depressive disorder. In some embodiments, a method comprises the administration to a patient in need thereof of a therapeutically effective amount of a compound of Formula (I)-(VI) for the treatment of anxiety and hysteria or anxiety and depression.

In certain embodiments, the present disclosure provides a method of treating a mental disorder, comprising administering to the subject a compound of the present disclosure (e.g., a compound of Formula (I)-(VI).

In certain embodiments, the present disclosure provides a method of inhibiting SERT, comprising administering to a mammal in need thereof a therapeutically effective amount of a pharmaceutical composition comprising a prodrug mesembranol. In certain embodiments, the mesembranol is (−) mesembranol. In certain embodiments, the mesembranol is (−) 6-epi-mesembranol. In certain embodiments, the composition comprises prodrug of mesembranol. In some embodiments, the compound is a compound of Formula (I). In some embodiments, the the prodrug of mesembranol is a compound of Formula (I) (e.g., a compound of Formula (I), (I-A), or (I-B). In some embodiments, the prodrug of mesembranol is a compound of Formula (II) (e.g., a compound of Formula (II), (II-A), or (II-B). In some embodiments, the prodrug of mesembranol is a compound of Formula (III) (e.g., a compound of Formula (III), (III-A), or (III-B). In some embodiments, the prodrug of mesembranol is a compound of Formula (IV) (e.g., a compound of Formula (IV), (IV-A), or (IV-B). In some embodiments, the prodrug of mesembranol is a compound of Formula (V) (e.g., a compound of Formula (V), (V-A), or (V-B). In some embodiments, the prodrug of mesembranol is a compound of Formula (VI) (e.g., a compound of Formula (VI), (VI-A), or (VI-B). In certain embodiments, pharmaceutical composition contains no more than about 10% of an alkaloid selected from (−) mesembrine, mesembrenone, mesembrenol, alone or in combination together. For example, the pharmaceutical composition may contain less than about 10%, less than about 5%, less than about 3%, less than about 2%, or less than about 1% by weight of the combination of alkaloids of (−) mesembrine and mesembrenone, or the combination of mesembrenone and mesembrenol, or the combination of (−) mesembrine, mesembrenone, and mesembrenol. In certain embodiments, the composition is substantially free of (−) mesembrine, mesembrenone, and mesembrenol.

In some embodiments, the compound disclosed herein is administered to the patient in a unit dose. In some embodiments, the compound disclosed herein is prescribed to a patient in an oral unit dose for such as a capsule or tablet once or more times per day. In some embodiments, a compound disclosed herein is administered to a patient for the treatment of a disease or condition for which a prodrug of mesembranol (e.g., compound of Formula (I)-(VI)) is safe and effective for treatment. In some embodiments, a method comprises the administration to a patient in need thereof of a therapeutically effective amount of a prodrug of mesembranol (e.g., compound of Formula (I)-(VI)) for the treatment of anxiety. In some embodiments, a method comprises the administration to a patient in need thereof of a therapeutically effective amount of a prodrug of mesembranol (e.g., compound of Formula (I)-(VI)) for the treatment of a disease selected from the group consisting of mild to moderate depression and major depressive episodes. In some embodiments, a method comprises the administration to a patient in need thereof of a therapeutically effective amount a prodrug of mesembranol (e.g., compound of Formula (I)-(VI)) for the treatment of a disease selected from the group consisting of psychological and psychiatric disorders where anxiety is present. In some embodiments, a method comprises the administration to a patient in need thereof of a therapeutically effective amount of a prodrug of mesembranol (e.g., compound of Formula (I)-(VI)) for the treatment of major depressive episodes. In some embodiments, a method comprises the administration to a patient in need thereof of a therapeutically effective amount a prodrug of mesembranol (e.g., compound of Formula (I)-(VI)) for the treatment of a disease selected from the group consisting of alcohol and drug dependence, bulimia nervosa, and obsessive-compulsive disorders. In some embodiments, an amount of from 20 micrograms to 2 milligrams of a prodrug of mesembranol (e.g., compound of Formula (I)-(VI)) is orally administered to a patient to treat the patient in need thereof. In some embodiments, an amount of from 20 micrograms to 2 milligrams of a prodrug of mesembranol (e.g., compound of Formula (I)-(VI)) is orally administered to a patient to treat the patient in need thereof.

### PHARMACEUTICAL COMPOSITIONS

In certain embodiments, the present application is directed to a pharmaceutical composition comprising an active pharmaceutical ingredient. In certain embodiments, the pharmaceutical composition comprises a compound as disclosed herein as the active pharmaceutical ingredient (API) and a pharmaceutically acceptable carrier comprising one or more excipients. In some embodiments, the pharmaceutical composition optionally further comprises an additional therapeutic compound (i.e., agent) with the pharmaceutically acceptable carrier. The pharmaceutical composition can be a medicament.

Pharmaceutically acceptable carriers include those known in the art. The choice of a pharmaceutically acceptable carrier can depend, for example, on the desired route of administration of the composition. A pharmaceutical composition (preparation) can be administered to a subject by any of a number of routes of administration including, for example, parenteral administration (e.g. intravenously, subcutaneously, or intramuscularly), oral administration (for example, tablets, and capsules); absorption through the oral mucosa (e.g., sublingually) or transdermally (for example as a patch applied to the skin) or topically (for example, as a cream, ointment or spray applied to the skin).

In some embodiments, pharmaceutical compositions comprising a prodrug of mesembranol (e.g., compound of Formula (I)-(VI)) or pharmaceutically acceptable salts thereof can be formulated for oral administration. For example, a compound provided herein can be combined with suitable compendial excipients to form an oral unit dosage form, such as a capsule or tablet, containing a target dose of a prodrug of mesembranol (e.g., compound of Formula (I)-(VI)). The drug product can be prepared by first manufacturing a prodrug of mesembranol (e.g., compound of Formula (I)-(VI)) as an active pharmaceutical ingredient (API), followed by roller compaction/milling with intragranular excipients and blending with extra granular excipients. A Drug Product can contain a prodrug of mesembranol (e.g., compound of Formula (I)-(VI)) as the API and excipient components in a tablet in a desired dosage strength. The blended material can be compressed to form tablets and then film coated. The excipients can be selected from materials appropriate for inclusion in a pharmaceutical composition for an intended purpose and route of delivery including providing a desired manufacturing and stability properties and/or desired in vivo characteristics or other properties to the pharmaceutical composition. In some embodiments, the pharmaceutical composition can include a prodrug of mesembranol (e.g., compound of Formula (I)-(VI)) as the API in combination with a filler (e.g., a form of microcrystalline cellulose), a dry binder or disintegrant (e.g., a cross-linked polymer), a glidant (e.g., colloidal silicon dioxide) and/or a lubricant (e.g., magnesium stearate). In some embodiments, the pharmaceutical composition can comprise a material such as an extended release or disintegrant involved in carrying or transporting the API pharmaceutical agent from one organ, or portion of the body, to another organ, or portion of the body of a subject, including materials to desirable control the absorption of the API in the intestine.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

To prepare solid dosage forms for oral administration, the active ingredient is mixed with one or more pharmaceutically acceptable carriers, such as sodium citrate or dicalcium phosphate, and/or any of the following: (1) fillers or extenders, (2) binders, (3) humectants, (4) disintegrating agents, (5) solution retarding agents, (6) absorption accelerators, (7) wetting agents, (8) absorbents, (9) lubricants, (10) complexing agents, and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using suitable excipients. The pharmaceutical compositions according to the present invention may contain conventional pharmaceutical carriers and/or auxiliary agents. In some embodiments, he pharmaceutical compositions according to the present invention may contain conventional carrier agents including a binder, a lubricant and/or a glidant selected from those products and materials generally used in pharmaceutical industry for preparation of pharmaceutical compositions for an intended route of administration.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

Liquid dosage forms useful for oral administration include pharmaceutically acceptable carriers and the active ingredient provided as a solid form for reconstitution prior to administration or as a liquid (e.g., solutions, suspensions, or emulsions). In addition to the active ingredient, a liquid dosage forms may contain inert diluents commonly used in the art. For example, formulations of pharmaceutically acceptable compositions for injection can include aqueous solutions such as water or physiologically buffered saline or other solvents or vehicles suitable for the intended route of administration. In some embodiments, the pharmaceutical composition is formulated for parenteral administration.

The therapeutically effective amount of a pharmaceutical composition can be determined by human clinical trials to determine the safe and effective dose for a patient with a relevant diagnosis. It is generally understood that the effective amount of the compound may vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the pharmaceutical composition at a dose and dose interval determined to be safe and effective for the patient.

The present disclosure includes the use of pharmaceutically acceptable salts of compounds of the invention in the compositions and methods of the present invention. Pharmaceutically-acceptable salts include, for example, acid-addition salts and base-addition salts. The acid that is added to a compound to form an acid-addition salt can be an organic acid or an inorganic acid. A base that is added to a compound to form a base-addition salt can be an organic base or an inorganic base. In some embodiments, a pharmaceutically-acceptable salt is a metal salt, in some embodiments, a pharmaceutically-acceptable salt is an ammonium salt. For example, a pharmaceutically acceptable acid addition salt can exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. Sec, e.g. “Principles of Neural Science”, McGraw-Hill Medical, New York, N. Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups, C₁-C₁₀ branched-chain alkyl groups, cycloalkyl (alicyclic) groups, alkyl-substituted cycloalkyl groups, and cycloalkyl-substituted alkyl groups. Preferably, the “alkyl” group refers to C₁-C₇ straight-chain alkyl groups or C₁-C₇ branched-chain alkyl groups. Most preferably, the “alkyl” group refers to C₁-C₃ straight-chain alkyl groups or C₁-C₃ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “haloalkyl” refers to an alkyl group substituted with at least one hydrogen atom on a carbon replaced by a halogen. Illustrative halogens include fluoro, chloro, bromo, and iodo. Illustrative haloalkyl groups include trifluoromethyl and 2,2,2-trifluoroethyl, etc.

The term “alkoxyalkyl” refers to an alkyl group substituted with an alkoxy group and may be represented by the general formula alkyl-O-alkyl.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

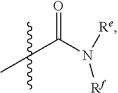

wherein R^{e} and R^{f} each independently represent a hydrogen or hydrocarbyl group, or R^{e} and R^{f} taken together with the N atom to which they are attached complete a heterocycle having from 4 to 8 atoms in the ring structure.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Preferably, the “alkoxy” group refers to C₁-C₇ straight-chain alkoxy groups or C₁-C₇ branched-chain alkoxy groups. Representative alkoxy groups include methoxy, ethoxy, propoxy, tert-butoxy and the like.

The term “aryloxy” refers to an aryl group having an oxygen attached thereto. Preferably, the “aryloxy” group refers to C₆-C₁₀ aryloxy groups or 5-7-membered heteroaryloxy groups. Representative aryloxy groups include phenoxy (C₆H₅—O—) and the like.

The terms “amine” and “amino” are art-recognized and refer to both unsubstituted and substituted amines and salts thereof, e.g., a moiety that can be represented by

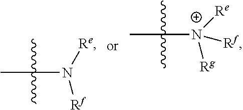

wherein R^{e}, R^{f}, and R⁸, each independently represent a hydrogen or a hydrocarbyl group, or R^{e} and R^{f} taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring, for example a phenyl. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

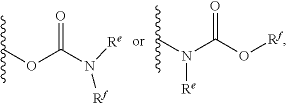

wherein R^{e} and R^{f} independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains six or fewer carbon atoms, preferably four or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

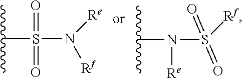

wherein R^{e} and R^{f} independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR^{e} or —SC(O)R^{e}
wherein R^{e} represents a hydrocarbyl.

The term “thioether”, as used herein, is equivalent to an ether, wherein the oxygen is replaced with a sulfur.

The term “urea” is art-recognized and may be represented by the general formula

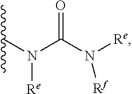

wherein R^{e} and R^{f} independently represent hydrogen or a hydrocarbyl.

The term “modulate” as used herein includes the inhibition or suppression of a function or activity (such as cell proliferation) as well as the enhancement of a function or activity.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. The mono- or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form mesembrine. Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs include using ester or phosphoramidate as biologically labile or cleavable (protecting) groups. The prodrugs of this disclosure are metabolized to produce mesembranol. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

### ADDITIONAL EMBODIMENTS

1. A compound of formula (I):

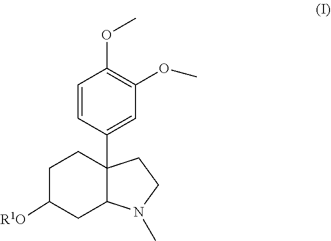

or a pharmaceutically acceptable salt thereof,
wherein:
R¹ is —C(O)NR²R³, —P(O)OR³OR⁴, —C(O)OR⁵, —C(O)R⁶, or —CH₂OC(O)R⁷;
R² is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy;
R³ is H, C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;
or R² and R³ taken together with the nitrogen atom to which they are attached form a 4-7 membered heterocycle, wherein each hydrogen atom in the 4-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;
R⁴ is H, C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;
or R³ and R⁴ taken together with the —O—P(O)—O— to which they are attached form a 5-7 membered heterocycle, wherein each hydrogen atom in the 5-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;
R⁵ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy;
R⁶ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, —OC(O)C₁-C₆ alkyl, nitro, —N(C₁-C₃ alkyl)₂, —NH₂, —N(H)C₁-C₃ alkyl, C₁-C₃ haloalkyl, —COOH, cyano, phenyl, or phenoxy;
R⁷ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy; and
n is 1 or 2.
2. The compound of embodiment 1, wherein the compound is of formula (I-A) or (I-B):

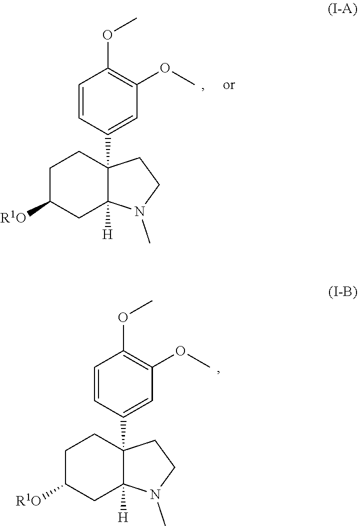

or a pharmaceutically acceptable salt thereof.
3. The compound of embodiment 1, wherein the compound is of formula (I-A) or (I-B):

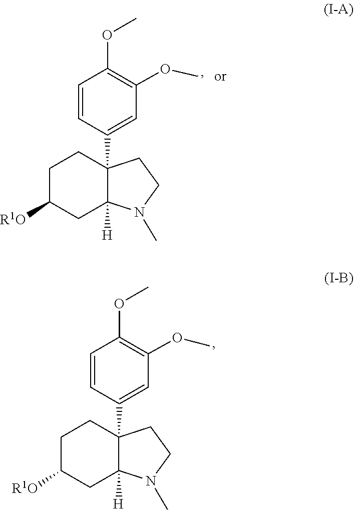

or a pharmaceutically acceptable salt thereof, wherein the compound has the absolute stereochemistry shown.
4. The compound of any one of embodiments 1-3, wherein R¹ is —C(O)NR²R³.
5. The compound of embodiment 4, wherein R² is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl.
6. The compound of embodiment 4 or 5, wherein R³ is C₁-C₆ alkyl, phenyl, —(CH₂O)ₙ—C(O)OC₁-C₆ alkyl, or —(CH₂O)ₙ—C(O)C₁-C₆ alkyl.
7. The compound of embodiment 4 or 5, wherein R³ is H.
8. The compound of embodiment 4, wherein each of R² and R³ is C₁-C₆ alkyl such as methyl.
9. The compound of embodiment 4, wherein R² and R³ taken together with the nitrogen atom to which they are attached form a 4-7 membered heterocycle.
10. The compound of any one of embodiments 1-3, wherein R¹ is —P(O)OR³OR⁴.
11. The compound of embodiment 10, wherein each of R³ and R⁴ is independently H, C₁-C₆ alkyl, phenyl, —(CH₂O)ₙ—C(O)OC₁-C₆ alkyl, or —(CH₂O)ₙ—C(O)C₁-C₆ alkyl.
12. The compound of embodiment 10, wherein R⁴ is C₁-C₆ alkyl, phenyl, —(CH₂O)ₙ—C(O)OC₁-C₆ alkyl, or —(CH₂O)ₙ—C(O)C₁-C₆ alkyl.
13. The compound of any one of embodiments 10-12, wherein n is 1.
14. The compound of embodiment 10, wherein each of R³ and R⁴ is H.
15. The compound of embodiment 10, wherein R³ and R⁴ taken together with the —O—P(O)—O— to which they are attached form a 5-7 membered heterocycle.
16. The compound of any one of embodiments 1-3, wherein R¹ is —C(O)OR⁵.
17. The compound of embodiment 16, wherein R⁵ is C₁-C₆ alkyl.
18. The compound of embodiment 16, wherein R⁵ is ethyl.
19. The compound of any one of embodiments 1-3, wherein R¹ is —C(O)R⁶.
20. The compound of embodiment 19, wherein R⁶ is C₁-C₆ alkyl or phenyl.
21. The compound of embodiment 19, wherein R⁶ is C₁-C₆ alkyl, such as t-butyl, isopropyl, or propyl.
22. The compound of embodiment 19, wherein R⁶ is phenyl.
23. The compound of any one of embodiments 1-3, wherein R¹ is —CH₂OC(O)R⁷.
24. The compound of embodiment 23, wherein R⁷ is C₁-C₆ alkyl.
25. The compound of embodiment 23, wherein R⁷ is unsubstituted C₁-C₆ alkyl.
26. The compound of embodiment 23, wherein R⁷ is isopropyl, propyl, or t-butyl.
27. The compound of embodiment 1, selected from:

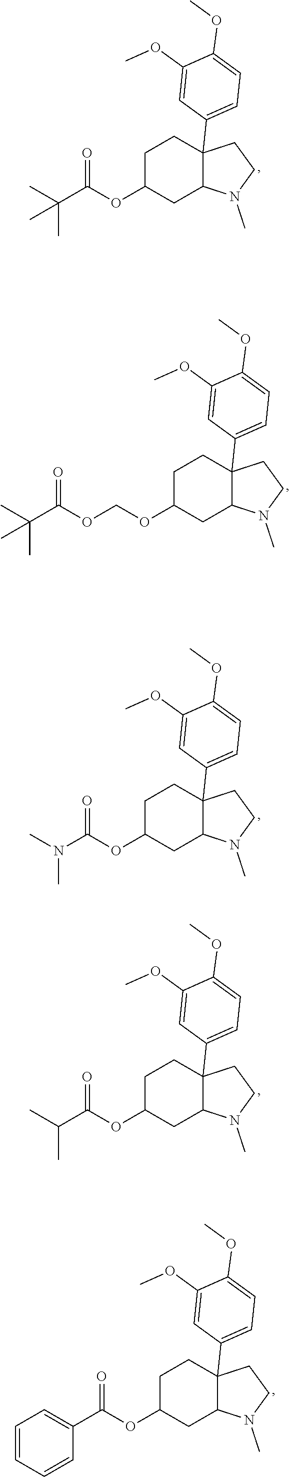 [SMILES: COc1ccc(C23CCC(OC(=O)C(C)(C)C)CC2N(C)CC3)cc1OC ; COc1ccc(C23CCC(OCOC(=O)C(C)(C)C)CC2N(C)CC3)cc1OC ; COc1ccc(C23CCC(OC(=O)N(C)C)CC2N(C)CC3)cc1OC ; COc1ccc(C23CCC(OC(=O)C(C)C)CC2N(C)CC3)cc1OC ; COc1ccc(C23CCC(OC(=O)c4ccccc4)CC2N(C)CC3)cc1OC]

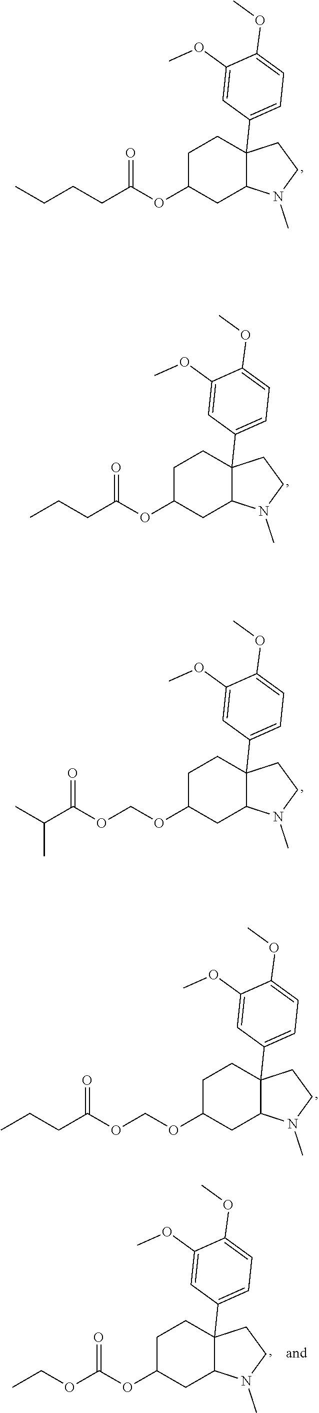 [SMILES: CCCCC(=O)OC1CCC2(c3ccc(OC)c(OC)c3)CCN(C)C2C1 ; CCCC(=O)OC1CCC2(c3ccc(OC)c(OC)c3)CCN(C)C2C1 ; COc1ccc(C23CCC(OCOC(=O)C(C)C)CC2N(C)CC3)cc1OC ; CCCC(=O)OCOC1CCC2(c3ccc(OC)c(OC)c3)CCN(C)C2C1 ; CCOC(=O)OC1CCC2(c3ccc(OC)c(OC)c3)CCN(C)C2C1]

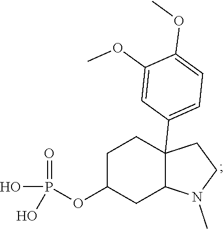 [SMILES: COc1ccc(C23CCC(OP(=O)(O)O)CC2N(C)CC3)cc1OC]

or a pharmaceutically acceptable salt thereof.
28. The compound of embodiment 1, selected from:

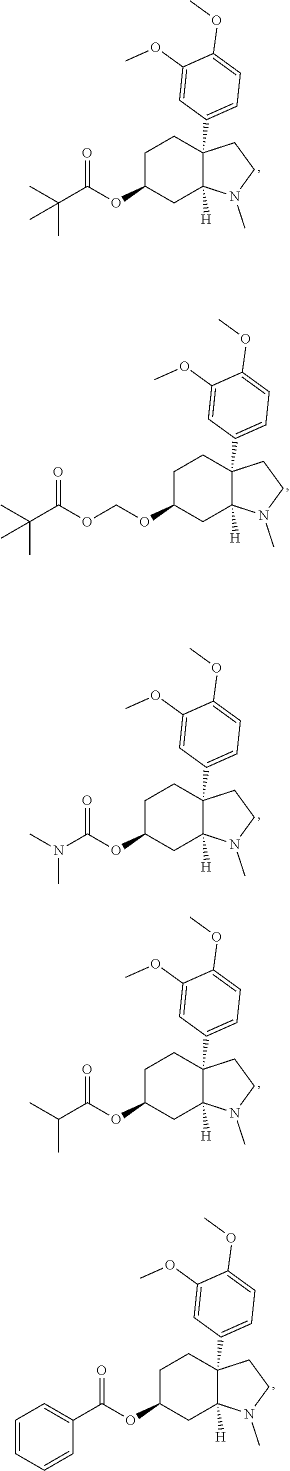 [SMILES: COc1ccc([C@@]23CC[C@H](OC(=O)C(C)(C)C)C[C@@H]2N(C)CC3)cc1OC ; COc1ccc([C@@]23CC[C@H](OCOC(=O)C(C)(C)C)C[C@@H]2N(C)CC3)cc1OC ; COc1ccc([C@@]23CC[C@H](OC(=O)N(C)C)C[C@@H]2N(C)CC3)cc1OC ; COc1ccc([C@@]23CC[C@H](OC(=O)C(C)C)C[C@@H]2N(C)CC3)cc1OC ; COc1ccc([C@@]23CC[C@H](OC(=O)c4ccccc4)C[C@@H]2N(C)CC3)cc1OC]

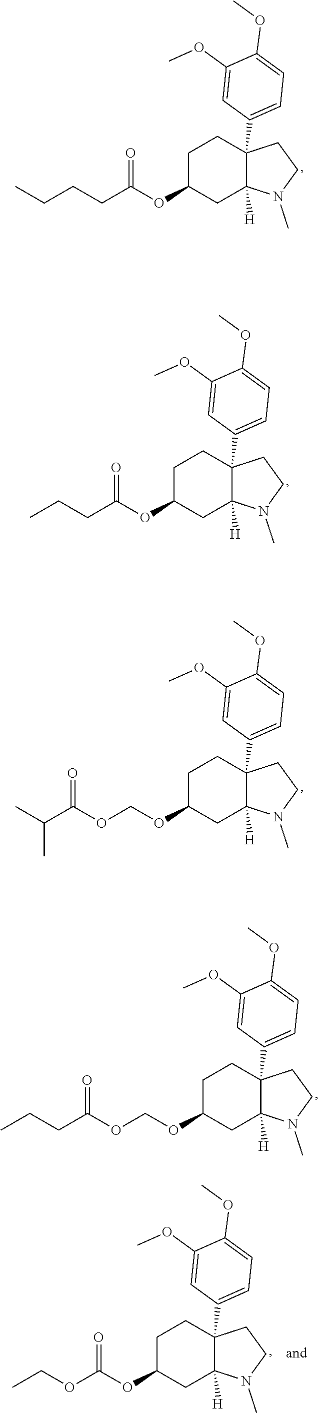 [SMILES: CCCCC(=O)O[C@H]1CC[C@@]2(c3ccc(OC)c(OC)c3)CCN(C)[C@H]2C1 ; CCCC(=O)O[C@H]1CC[C@@]2(c3ccc(OC)c(OC)c3)CCN(C)[C@H]2C1 ; COc1ccc([C@@]23CC[C@H](OCOC(=O)C(C)C)C[C@@H]2N(C)CC3)cc1OC ; CCCC(=O)OCO[C@H]1CC[C@@]2(c3ccc(OC)c(OC)c3)CCN(C)[C@H]2C1 ; CCOC(=O)O[C@H]1CC[C@@]2(c3ccc(OC)c(OC)c3)CCN(C)[C@H]2C1]

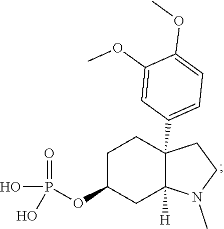 [SMILES: COc1ccc([C@@]23CC[C@H](OP(=O)(O)O)C[C@@H]2N(C)CC3)cc1OC]

or a pharmaceutically acceptable salt thereof.
29. The compound of embodiment 1, selected from:

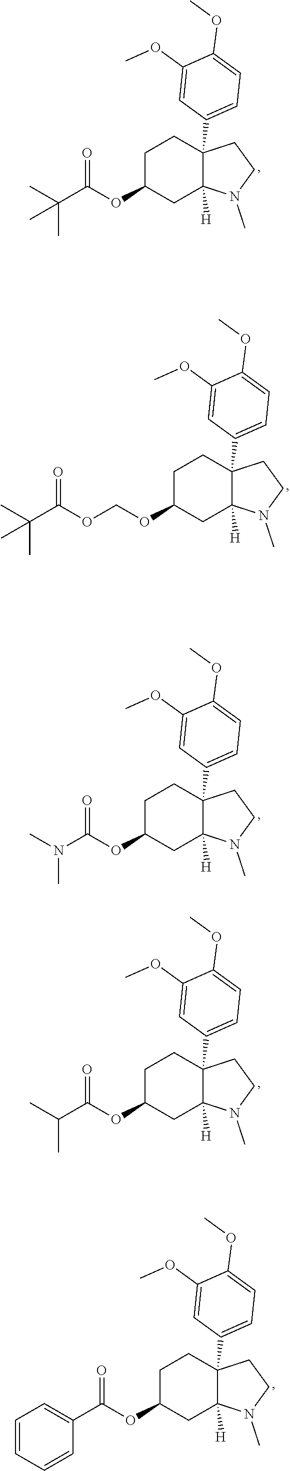 [SMILES: COc1ccc([C@@]23CC[C@H](OC(=O)C(C)(C)C)C[C@@H]2N(C)CC3)cc1OC ; COc1ccc([C@@]23CC[C@H](OCOC(=O)C(C)(C)C)C[C@@H]2N(C)CC3)cc1OC ; COc1ccc([C@@]23CC[C@H](OC(=O)N(C)C)C[C@@H]2N(C)CC3)cc1OC ; COc1ccc([C@@]23CC[C@H](OC(=O)C(C)C)C[C@@H]2N(C)CC3)cc1OC ; COc1ccc([C@@]23CC[C@H](OC(=O)c4ccccc4)C[C@@H]2N(C)CC3)cc1OC]

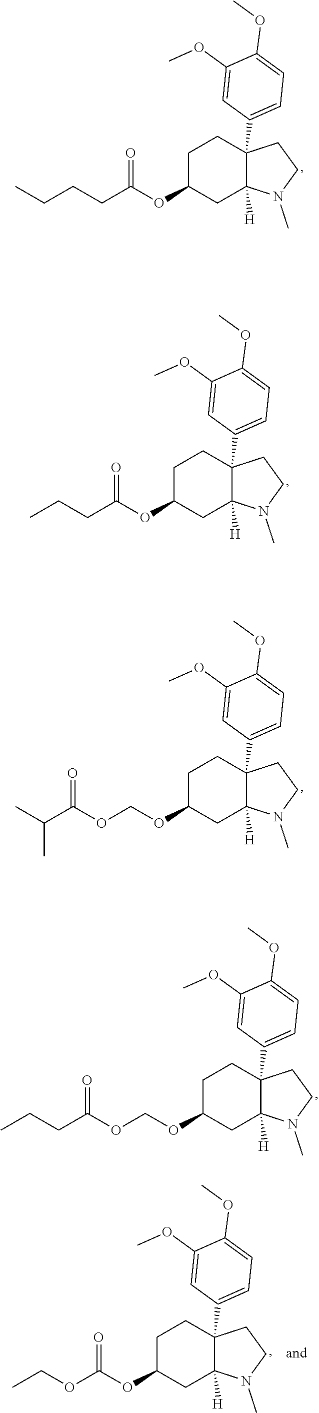 [SMILES: CCCCC(=O)O[C@H]1CC[C@@]2(c3ccc(OC)c(OC)c3)CCN(C)[C@H]2C1 ; CCCC(=O)O[C@H]1CC[C@@]2(c3ccc(OC)c(OC)c3)CCN(C)[C@H]2C1 ; COc1ccc([C@@]23CC[C@H](OCOC(=O)C(C)C)C[C@@H]2N(C)CC3)cc1OC ; CCCC(=O)OCO[C@H]1CC[C@@]2(c3ccc(OC)c(OC)c3)CCN(C)[C@H]2C1 ; CCOC(=O)O[C@H]1CC[C@@]2(c3ccc(OC)c(OC)c3)CCN(C)[C@H]2C1]

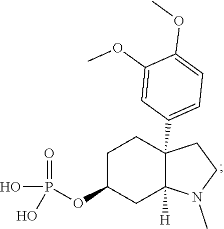 [SMILES: COc1ccc([C@@]23CC[C@H](OP(=O)(O)O)C[C@@H]2N(C)CC3)cc1OC]

or a pharmaceutically acceptable salt thereof; wherein the compound has the absolute stercochemistry shown.
30. The compound of embodiment 1, selected from:

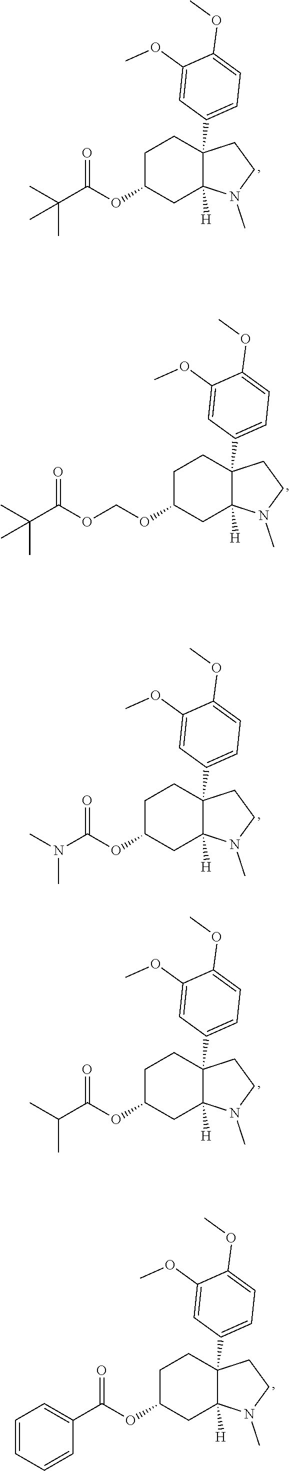 [SMILES: COc1ccc([C@@]23CC[C@@H](OC(=O)C(C)(C)C)C[C@@H]2N(C)CC3)cc1OC ; COc1ccc([C@@]23CC[C@@H](OCOC(=O)C(C)(C)C)C[C@@H]2N(C)CC3)cc1OC ; COc1ccc([C@@]23CC[C@@H](OC(=O)N(C)C)C[C@@H]2N(C)CC3)cc1OC ; COc1ccc([C@@]23CC[C@@H](OC(=O)C(C)C)C[C@@H]2N(C)CC3)cc1OC ; COc1ccc([C@@]23CC[C@@H](OC(=O)c4ccccc4)C[C@@H]2N(C)CC3)cc1OC]

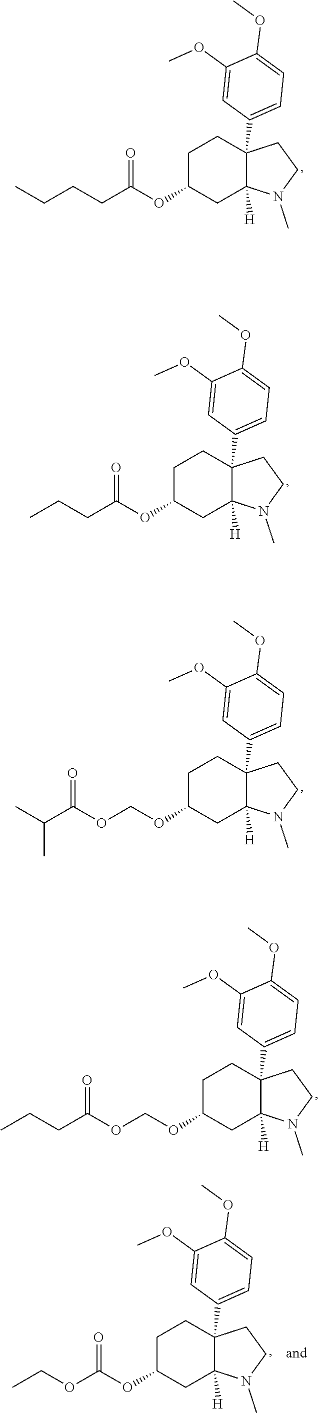 [SMILES: CCCCC(=O)O[C@@H]1CC[C@@]2(c3ccc(OC)c(OC)c3)CCN(C)[C@H]2C1 ; CCCC(=O)O[C@@H]1CC[C@@]2(c3ccc(OC)c(OC)c3)CCN(C)[C@H]2C1 ; COc1ccc([C@@]23CC[C@@H](OCOC(=O)C(C)C)C[C@@H]2N(C)CC3)cc1OC ; CCCC(=O)OCO[C@@H]1CC[C@@]2(c3ccc(OC)c(OC)c3)CCN(C)[C@H]2C1 ; CCOC(=O)O[C@@H]1CC[C@@]2(c3ccc(OC)c(OC)c3)CCN(C)[C@H]2C1]

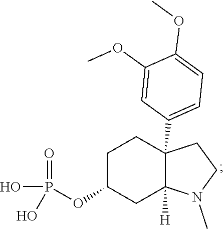 [SMILES: COc1ccc([C@@]23CC[C@@H](OP(=O)(O)O)C[C@@H]2N(C)CC3)cc1OC]

or a pharmaceutically acceptable salt thereof.
31. The compound of embodiment 1, selected from:

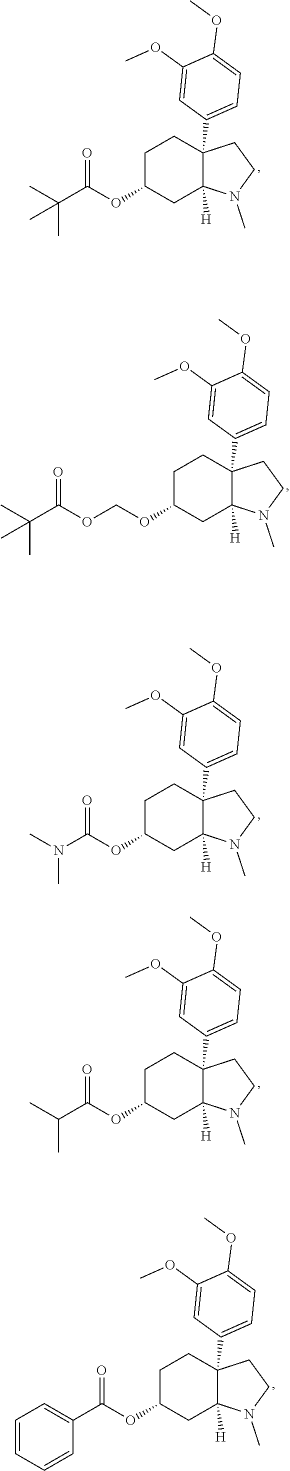 [SMILES: COc1ccc([C@@]23CC[C@@H](OC(=O)C(C)(C)C)C[C@@H]2N(C)CC3)cc1OC ; COc1ccc([C@@]23CC[C@@H](OCOC(=O)C(C)(C)C)C[C@@H]2N(C)CC3)cc1OC ; COc1ccc([C@@]23CC[C@@H](OC(=O)N(C)C)C[C@@H]2N(C)CC3)cc1OC ; COc1ccc([C@@]23CC[C@@H](OC(=O)C(C)C)C[C@@H]2N(C)CC3)cc1OC ; COc1ccc([C@@]23CC[C@@H](OC(=O)c4ccccc4)C[C@@H]2N(C)CC3)cc1OC]

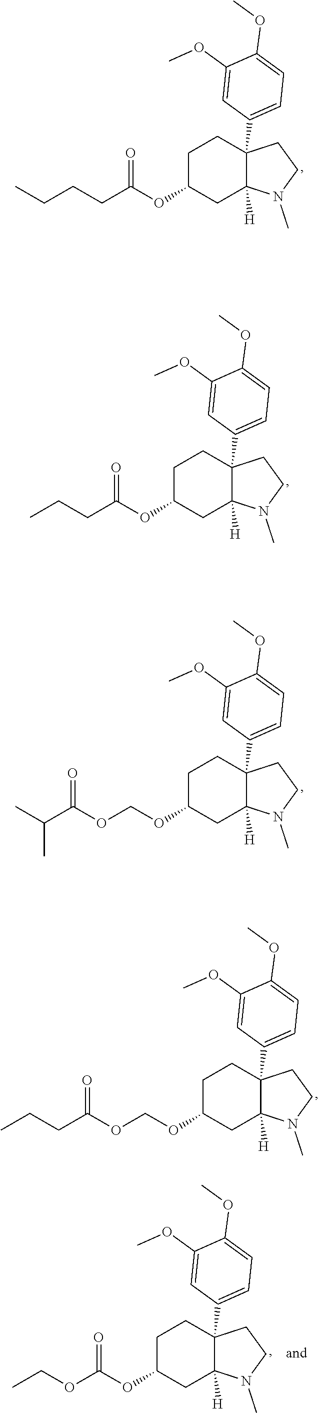 [SMILES: CCCCC(=O)O[C@@H]1CC[C@@]2(c3ccc(OC)c(OC)c3)CCN(C)[C@H]2C1 ; CCCC(=O)O[C@@H]1CC[C@@]2(c3ccc(OC)c(OC)c3)CCN(C)[C@H]2C1 ; COc1ccc([C@@]23CC[C@@H](OCOC(=O)C(C)C)C[C@@H]2N(C)CC3)cc1OC ; CCCC(=O)OCO[C@@H]1CC[C@@]2(c3ccc(OC)c(OC)c3)CCN(C)[C@H]2C1 ; CCOC(=O)O[C@@H]1CC[C@@]2(c3ccc(OC)c(OC)c3)CCN(C)[C@H]2C1]

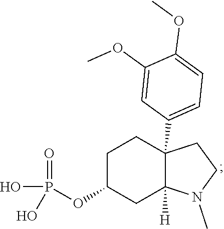 [SMILES: COc1ccc([C@@]23CC[C@@H](OP(=O)(O)O)C[C@@H]2N(C)CC3)cc1OC]

or a pharmaceutically acceptable salt thereof; wherein the compound has the absolute stercochemistry shown.
32. A compound of formula (II):

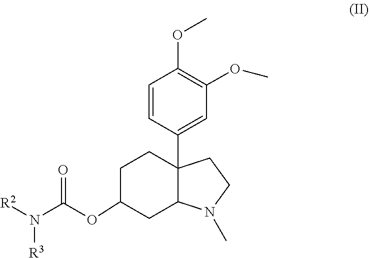

or a pharmaceutically acceptable salt thereof, wherein:
R² is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy;
R³ is H, C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;
or R² and R³ taken together with the nitrogen atom to which they are attached form a 4-7 membered heterocycle, wherein each hydrogen atom in the 4-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy; and
n is 1 or 2.
33. The compound of embodiment 32, wherein the compound is of formula (II-A) or (II-B):

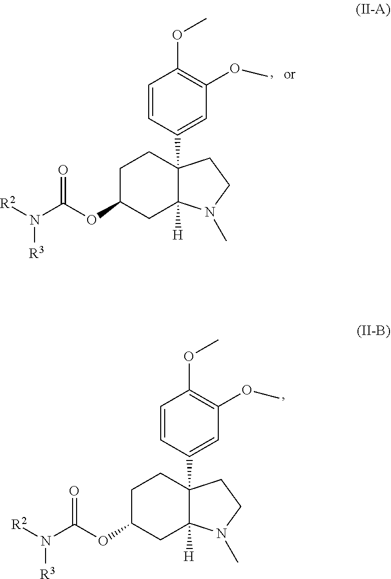

or a pharmaceutically acceptable salt thereof.
34. The compound of embodiment 32, wherein the compound is of formula (II-A) or (II-B):

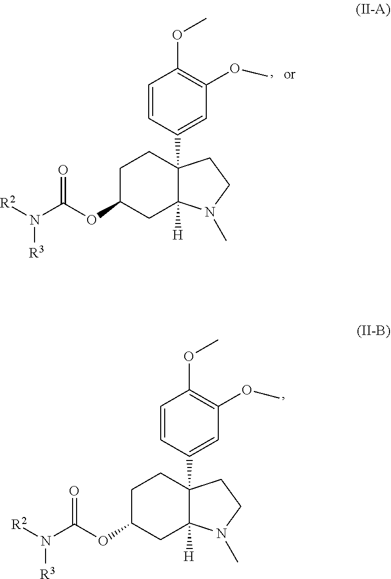

or a pharmaceutically acceptable salt thereof, wherein the compound has the absolute stereochemistry shown.
35. A compound of formula (III):

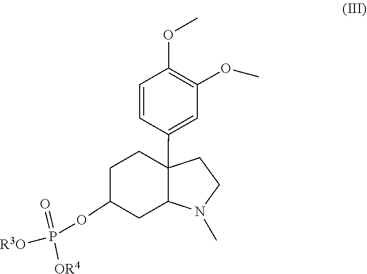

or a pharmaceutically acceptable salt thereof,
wherein:
R³ is H, C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;
R⁴ is H, C₁-C₆ alkyl, phenyl, —(CH₂O)ₙ—C(O)OC₁-C₆ alkyl, or —(CH₂O)ₙ—C(O)C₁-C₆ alkyl, wherein each hydrogen atom in C₁-C₆ alkyl is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;
or R³ and R⁴ taken together with the —O—P(O)—O— to which they are attached form a 5-7 membered heterocycle, wherein each hydrogen atom in the 5-7 membered heterocycle is optionally substituted by halo, hydroxy, C₁-C₃ alkyl, C₁-C₃ alkoxy, C₃-C₁₀ cycloalkyl, phenyl, 5-7 membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy; and
n is 1 or 2.
36. The compound of embodiment 35, wherein the compound is of formula (III-A) or (III-B):

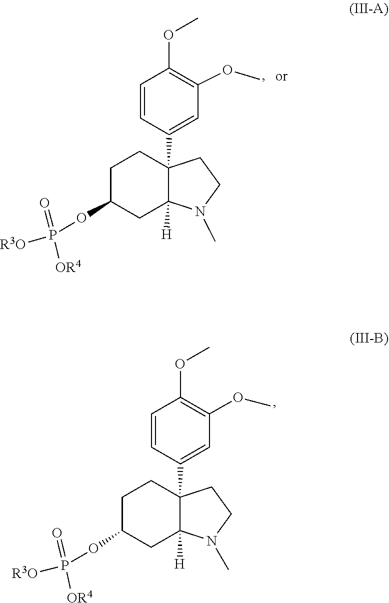

or a pharmaceutically acceptable salt thereof.
37. The compound of embodiment 35, wherein the compound is of formula (III-A) or (III-B):

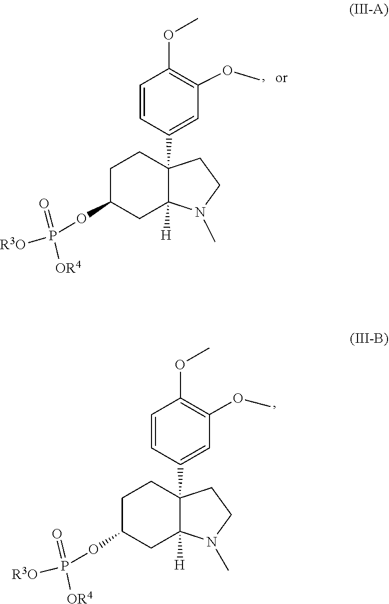

or a pharmaceutically acceptable salt thereof, wherein the compound has the absolute stereochemistry shown.
38. A compound of formula (IV):

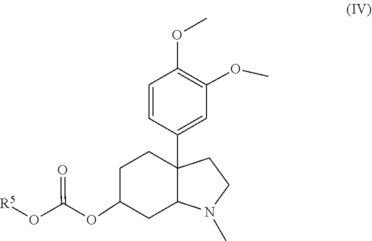

or a pharmaceutically acceptable salt thereof,
wherein:
R⁵ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy.
39. The compound of embodiment 38, wherein the compound is of formula (IV-A) or (IV-B):

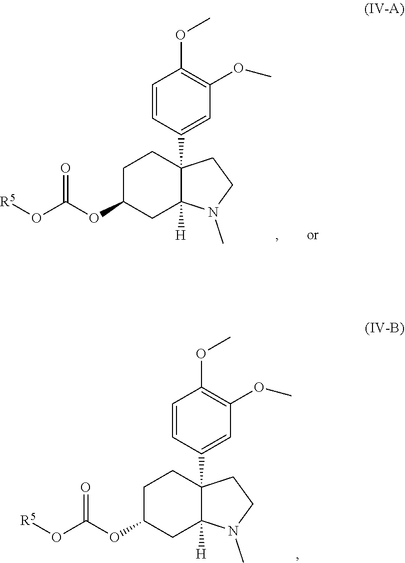

or a pharmaceutically acceptable salt thereof.
40. The compound of embodiment 38, wherein the compound is of formula (IV-A) or (IV-B):

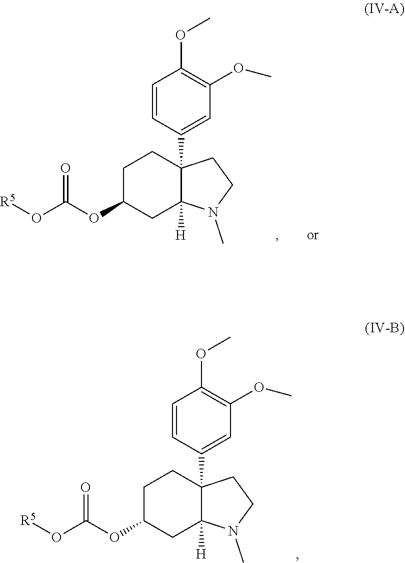

or a pharmaceutically acceptable salt thereof, wherein the compound has the absolute stereochemistry shown.
41. A compound of formula (V):

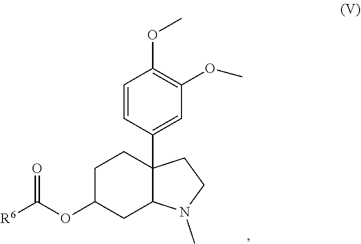

or a pharmaceutically acceptable salt thereof,
wherein:
R⁶ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, —OC(O)C₁-C₆ alkyl, nitro, —N(C₁-C₃ alkyl)₂, —NH₂, —N(H)C₁-C₃ alkyl, C₁-C₃ haloalkyl, —COOH, cyano, phenyl, or phenoxy.
42. The compound of embodiment 41, wherein the compound is of formula (V-A) or (V-B):

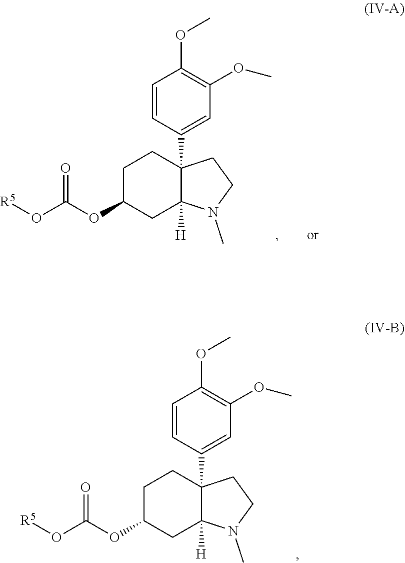

or a pharmaceutically acceptable salt thereof.
43. The compound of embodiment 41, wherein the compound is of formula (V-A) or (V-B):

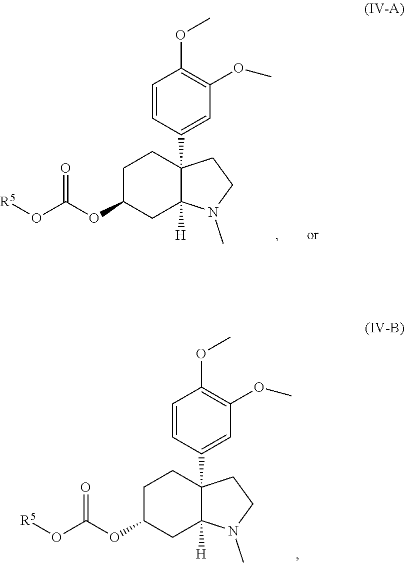

or a pharmaceutically acceptable salt thereof, wherein the compound has the absolute stereochemistry shown.
44. A compound of formula (VI):

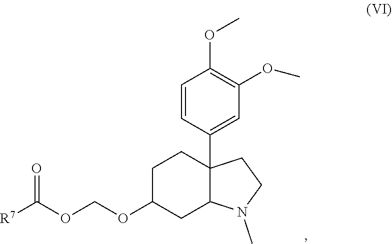

or a pharmaceutically acceptable salt thereof,
wherein:
R⁷ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy.
45. The compound of embodiment 44, wherein the compound is of formula (VI-A) or (VI-B):

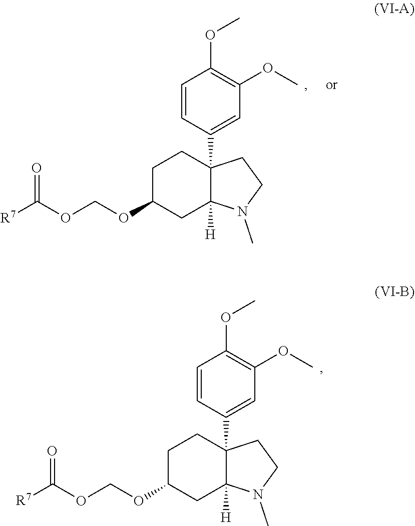

or a pharmaceutically acceptable salt thereof.
46. The compound of embodiment 44, wherein the compound is of formula (VI-A) or (VI-B):

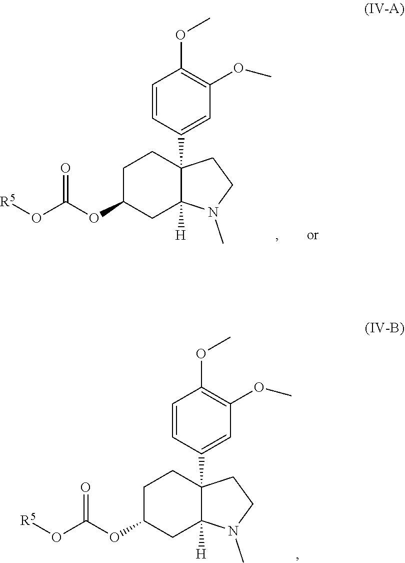

or a pharmaceutically acceptable salt thereof, wherein the compound has the absolute stereochemistry shown.
47. A pharmaceutical composition, comprising a compound of any one of embodiments 1-46; and a pharmaceutically acceptable excipient.
48. A method of treating a mental health disorder, comprising administering to a mammal in need thereof an effective amount of a compound of any one of embodiments 1-46.
49. The method of embodiment 48, wherein the mental health disorder is anxiety, stress, or depression.
50. The method of embodiment 49, wherein the mental health disorder is anxiety.
51. The method of embodiment 49, wherein the mental health disorder is stress.
52. The method of embodiment 49, wherein the mental health disorder is depression.
53. The method of any one of embodiments 48-52, wherein the mammal is a human.

### EXAMPLES

LC/MS spectra were obtained using Agilent 1200\G1956A or SHIMADZU LCMS-2020. Standard LC/MS conditions were as follows (running time 1.55 minutes):

Acidic condition: Mobile Phase A: 0.0375% TFA in water (v/v). Mobile Phase B: 0.01875% TFA in acetonitrile (v/v); Column: Kinetex EVO C18 30*2.1 mm, 5 μm.

Basic condition: Mobile Phase A: 0.025% NH₃·H₂O in water (v/v). Mobile Phase B: Acetonitrile; Column: Kinetex EVO C18 2.1×30 mm, 5 μm.

5-95AB_0.8 min

| Instrument | | SHIMADZU LCMS-2020; | | |
|---|---|---|---|---|
| Software | | LabSolution Version 5.97SP1 | | |
| | Column | Kinetex ® EVO C18 2.1 × 30 mm 5 um | | |
| | Mobile Phase | A: 0.0375% TFA in water (v/v) | | |
| | | B: 0.01875% TFA in Acetonitrile (v/v) | | |
| HPLC | Gradient | Time(min) | B(%) | Flow(mL/min) |
| | | 0.00 | 5.0 | 2.0 |
| | | 0.60 | 95.0 | 2.0 |
| | | 0.78 | 95.0 | 2.0 |
| | | 0.79 | 5.0 | 2.0 |
| | | 0.80 | 5.0 | 2.0 |

| | | |
|---|---|---|
| | Column Temp | 50° C. |
| | Detector | PDA (220 nm & 254 nm) |
| MS | Ionization source | ESI |
| | Drying Gas | N2 |
| | Drying Gas Flow | 15(L/min) |
| | DL Voltage | 120(v) |
| | Qarray DC Voltage | 20(V) |
| | MS Polarity | Positive |
| | MS Mode | Scan |
| | Mass range | 100-1000 |

Table of Abbreviations

| | |
|---|---|
| Ac | Acetyl |
| ACN | Acetonitrile |
| br | Broad |
| C. | Celsius |
| δ | Chemical shift |
| d | Doublet |
| DCM | Dichloromethane |
| de | Diastereomeric excess |
| DIBAL-H | Diisobutyl aluminum hydride |
| DMAP | 4-Dimethylaminopyridine |
| DMF | N,N-Dimethyformamide |
| ESI | Electrospray ionization |
| Et | Ethyl |
| FA | Formic acid |
| HPLC | High performance liquid chromatography |
| hr | Hours |
| Hz | Hertz |
| IPA | Isopropyl alcohol |
| J | Coupling constant |
| LC-MS | Liquid chromatography-mass spectrometry |
| m | Multiplet |
| M | Molar |
| m/z | Mass to charge ratio |
| Me | Methyl |
| min | Minutes |
| mol | Moles |
| NMR | Nuclear magnetic resonance |
| Psi | pound-force per square inch |
| Pr | propyl |
| q | Quartet |
| s | Singlet |
| t | Triplet |
| t-Bu | Tertiary butyl |
| TEA | Triethylamine |
| THF | Tetrahydrofuran |
| TMS | Trimethylsilyl |

### Summary of Mesembrine and Mesembranol Compound Designations

| Compound | (−) enantiomer |
|---|---|
| Mesembrine | Compound 001 |
| Mesembranol | Compound 018 |
| 6-epi-mesembranol | Compound 019 |

### Example 1: Synthesis of (+/−)-Mesembrine

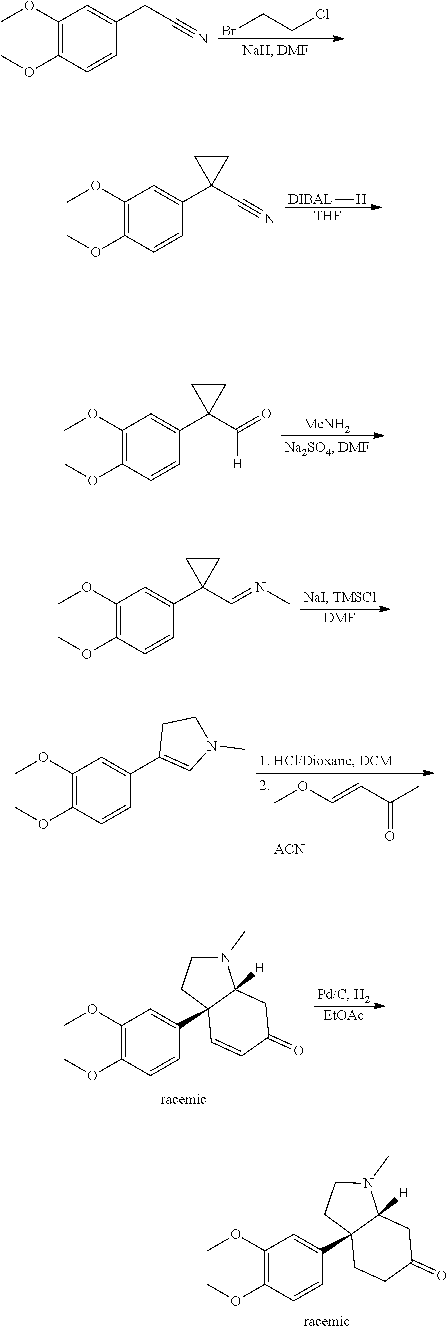

### Step 1: Synthesis of 1-(3,4-dimethoxyphenyl)cyclopropane-1-carbonitrile

To a solution of 2-(3,4-dimethoxyphenyl) acetonitrile (20 g, 112 mmol) in DMF (93 mL) was added NaH (18.0 g, 451 mmol, 60% purity) in portions. The mixture was allowed to stir at 25° C. for 20 min. 1-Bromo-2-chloro-ethane (16.1 g, 112 mmol) was added, and the mixture was allowed to stir at 25° C. for 16 hr. The reaction was quenched by the addition of a MeOH/water mixture (1:1; 1000 mL) and the resulting solution was extracted with EtOAc (3×500 mL). The organic solutions were combined, washed with water (4×500 mL) and brine (1×200 mL) and dried over (Na₂SO₄). The solution was filtered and the solvent was evaporated under reduced pressure. The resulting solid was purified by column chromatography (SiO₂, Petroleum ether/EtOAc=10/1 to 3/1) to give 1-(3,4-dimethoxyphenyl)cyclopropane-1-carbonitrile (15 g, 65%) as yellow oil. 1H NMR (400 MHZ, CDCl₃) δ 6.88 (s, 1H), 6.82 (d, J=1.2 Hz, 2H), 3.91 (s, 3H), 3.88 (s, 3H), 1.68-1.65 (m, 2H), 1.35 (d, J=2.4 Hz, 2H).

### Step 2: Synthesis of 1-(3,4-dimethoxyphenyl)cyclopropane-1-carbaldehyde

To a solution of 1-(3,4-dimethoxyphenyl)cyclopropane-1-carbonitrile (11 g, 54.1 mmol) in THF (160 mL) was added DIBAL-H (1M in toluene, 81.2 mL). The mixture was allowed to stir at 25° C. for 3 hr and then the reaction was cautiously quenched by addition of aqueous 2M HCl. The solution was extracted with DCM (3×200 mL). The organic solutions were combined, washed with water (2×200 mL) and brine (2×200 mL), and then dried over Na₂SO₄ to give 1-(3,4-dimethoxyphenyl)cyclopropane-1-carbaldehyde (9.6 g, 85%) as yellow oil. LC-MS (ESI+) m/z 207.0 (M+H)⁺. ¹H NMR (400 MHZ, CDCl₃) δ 9.26 (s, 1H), 6.94-6.61 (m, 3H), 3.89 (d, J=2.8 Hz, 6H), 1.61-1.52 (m, 2H), 1.42-1.37 (m, 2H)

### Step 3: Synthesis of (Z)-1-(1-(3,4-dimethoxyphenyl)cyclopropyl)-N-methylmethanimine

To a solution of 1-(3,4-dimethoxyphenyl)-cyclopropanecarbaldehyde (5.0 g, 24.2 mmol) in DCM (50 mL) was added MeNH₂ (2 M, 121 mL) and Na₂SO₄ (15.5 g, 109 mmol, 11.0 mL). The mixture was allowed to stir at 25° C. for 16 hr. The reaction mixture was filtered and the filtrate was concentrated under reduced pressure to give (Z)-1-(1-(3,4-dimethoxyphenyl)cyclopropyl)-N-methylmethanimine (5.1 g, 99%) as white solid. LC-MS (ESI⁺) m/z 219.9 (M+H)⁺; 1H NMR (400 MHZ, CDCl₃) δ 7.55 (q, J=1.2 Hz, 1H), 6.93-6.77 (m, 3H), 3.88 (d, J=7.2 Hz, 6H), 3.24 (d, J=1.6 Hz, 3H), 1.29-1.23 (m, 2H), 1.18-1.12 (m, 2H).

### Step 4: Synthesis of 4-(3,4-dimethoxyphenyl)-1-methyl-2,3-dihydro-1H-pyrrole

To a solution of (Z)-1-(1-(3,4-dimethoxyphenyl)cyclopropyl)-N-methylmethanimine (5.4 g, 24.6 mmol) in DMF (19 mL) was added NaI (366 mg, 2.44 mmol) and TMSCl (267 mg, 2.46 mmol). The mixture was allowed to stir at 90° C. for 3 hr. The reaction mixture was diluted with water (100 mL) and extracted with EtOAc (100 mL×3). The organic solutions were combined, washed with water and brine, dried over Na₂SO₄, and filtered. The filtrate was concentrated under reduced pressure to give 4-(3,4-dimethoxyphenyl)-1-methyl-2,3-dihydro-1H-pyrrole (6.25 g, 80%) as yellow oil. LC-MS (ESI⁺) m/z 220.0 (M+H)⁺. ¹H NMR (400 MHZ, CDCl₃) δ 6.90-6.66 (m, 3H), 6.31 (t, J=1.6 Hz, 1H), 3.95-3.80 (m, 6H), 3.18-3.11 (m, 2H), 2.79 (dt, J=1.2, 9.0 Hz, 2H), 2.65 (s, 3H).

### Step 5: Synthesis of rac-3a-(3,4-dimethoxyphenyl)-1-methyl-1,2,3,3a,7,7a-hexahydro-6H-indol-6-one

4-(3,4-Dimethoxyphenyl)-1-methyl-2,3-dihydro-1H-pyrrole (6.25 g, 28.5 mmol) was dissolved in DCM (100 mL). To this solution was added HCl (1M in dioxane, 25 mL, 100 mmol). The mixture was evaporated to dryness and then dissolved in ACN (90 mL). To this solution was added (E)-4-methoxybut-3-en-2-one (4.28 g, 42.7 mmol). The reaction mixture was allowed to stir at 90° C. for 16 hr. The reaction mixture was filtered and the filtrate was concentrated under reduced pressure. The residue was purified by HPLC (column: Phenomenex luna C18 (250*70 mm, 10 um); mobile phase: [water(NH₄HCO₃)-ACN]; B %: 22%-52%, 20 min). The eluant was acidified with aq. HCl to give rac-3a-(3,4-dimethoxyphenyl)-1-methyl-1,2,3,3a,7,7a-hexahydro-6H-indol-6-one (3.0 g, 30%) as a white solid. LC-MS (ESI⁺) m/z 288.3 (M+H)⁺. ¹H NMR (400 MHZ, CDCl₃) δ 6.90-6.88 (m, 1H), 6.87-6.83 (m, 2H), 6.74 (dd, J=2.0, 10.1 Hz, 1H), 6.11 (d, J=10.0 Hz, 1H), 3.89 (d, J=4.0 Hz, 6H), 3.33 (dt, J=2.4, 8.8 Hz, 1H), 2.69-2.66 (m, 1H), 2.58-2.51 (m, 2H), 2.50-2.41 (m, 2H), 2.33 (s, 3H), 2.27-2.18 (m, 1H)

### Step 6: Synthesis of rac-3a-(3,4-dimethoxyphenyl)-1-methyloctahydro-6H-indol-6-one

A mixture of rac-3a-(3,4-dimethoxyphenyl)-1-methyl-1,2,3,3a,7,7a-hexahydro-6H-indol-6-one (12.0 g, 43.9 mmol) and 10% Pd/C (300 mg) in EtOAc (120 mL) was degassed and then purged with H₂ for 3 times. The mixture was allowed to stir at 25° C. for 2 hr under 15 psi H₂. The reaction mixture was filtered and the filtrate was concentrated in vacuo to give rac-3a-(3,4-dimethoxyphenyl)-1-methyloctahydro-6H-indol-6-one (10 g, 80%) as brown oil. LC-MS (ESI⁺) m/z 290.4 (M+H)⁺. ¹H NMR (400 MHZ, CDCl₃) δ 6.99-6.89 (m, 2H), 6.89-6.84 (m, 1H), 3.91 (d, J=7.6 Hz, 6H), 3.20-3.11 (m, 1H), 2.97 (t, J=3.6 Hz, 1H), 2.69-2.56 (m, 2H), 2.51-2.31 (m, 5H), 2.27-2.18 (m, 3H), 2.18-2.07 (m, 2H).

### Example 2: Chiral Resolution of rac-3a-(3,4-dimethoxyphenyl)-1-methyloctahydro-6H-indol-6-one to give (−) mesembrine 001

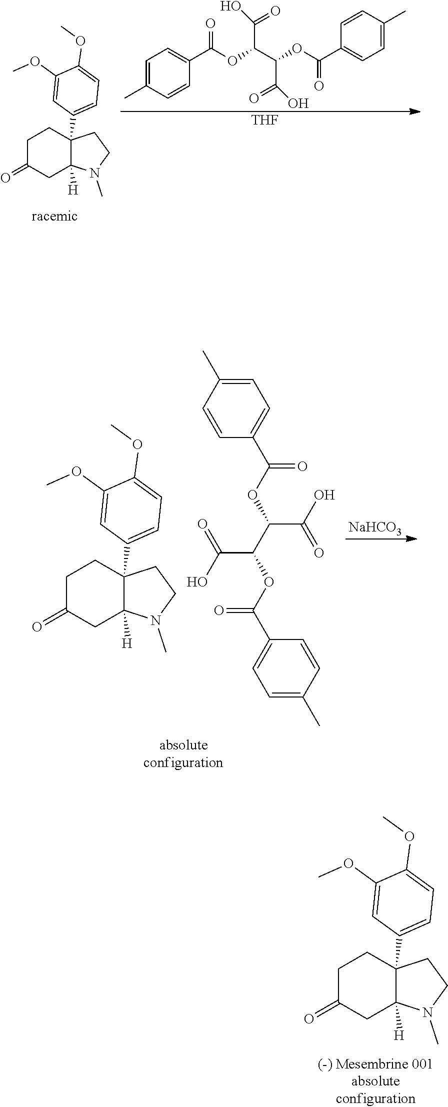

To a solution of rac-3a-(3,4-dimethoxyphenyl)-1-methyloctahydro-6H-indol-6-one (28.0 g, 85.1 mmol) in THF (1400 mL) was added (2S,3S)-2,3-bis[(4-methylbenzoyl)oxy]butanedioic acid (19.7 g, 51.1 mmol). The suspension was allowed to stir at 25° C. for 16 hr and then filtered. The solid was dried in vacuo and then triturated 3 times with THF at 25° C. for 16 hr. The resulting solid was filtered and then added to a saturated sodium bicarbonate solution (500 mL). The mixture was extracted with EtOAc (500 mL). The organic solutions were combined, dried over Na₂SO₄, and filtered. The filtrate was concentrated in vacuo to give (−)-Mesembrine 001 (7.50 g, 95% ee) as a yellow gum. LC-MS (ESI⁺) m/z 290.6 (M+H)⁺. ¹H NMR (400 MHZ, CDCl₃) δ 6.89-6.81 (m, 2H), 6.80-6.75 (m, 1H), 3.82 (d, J=8.0 Hz, 6H), 3.11-3.03 (m, 1H), 2.88 (t, J=3.6 Hz, 1H), 2.59-2.48 (m, 2H), 2.43-2.32 (m, 1H), 2.31-2.21 (m, 4H), 2.20-2.09 (m, 3H), 2.08 (br s, 2H).

### Example 3: Synthesis of (3aS,6S,7aS)-3a-(3,4-dimethoxyphenyl)-1-methyloctahydro-1H-indol-6-yl dimethylcarbamate (101)

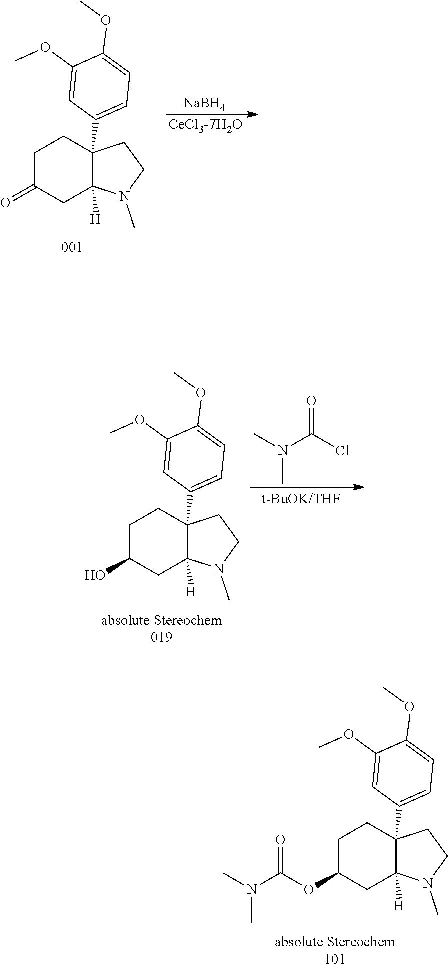

### Step 1: Synthesis of (3aS,6S,7aS)-3a-(3,4-dimethoxyphenyl)-1-methyloctahydro-1H-indol-6-ol (019)

To a solution of 001 (2.00 g, 6.91 mmol) and CeCl₃·7H₂O (3.09 g, 8.29 mmol, 788 uL) in MeOH (80 mL) was added NaBH₄ (1.57 g, 41.4 mmol). The mixture was allowed to stir at 0° C. for 2 hr. The reaction mixture was added into 50 mL NH₄Cl aqueous solution, the organic and aqueous layers were separated, and the aqueous solution was extracted with DCM (50 mL×3). The organic solutions were combined, dried over anhydrous Na₂SO₄, filtered and concentrated under reduced pressure. The residue was purified by prep-HPLC (column: Welch Xtimate C18 150*25 mm*5 um; mobile phase: [water(NH₃H₂O)-ACN]; B %: 28%-58%, 8 min) to give (3aS,6S,7aS)-3a-(3,4-dimethoxyphenyl)-1-methyloctahydro-1H-indol-6-ol (019) (730 mg, 37%) as white oil. ¹H NMR (400 MHZ, CDCl₃) δ 6.95-6.88 (m, 2H), 6.86-6.80 (m, 1H), 3.95 (s, 1H), 3.90 (d, J=6.8 Hz, 6H), 3.46-3.35 (m, 1H), 2.93 (s, 1H), 2.50 (s, 3H), 2.45-2.29 (m, 2H), 2.19 (dd, J=2.4, 14.9 Hz, 1H), 2.01-1.82 (m, 2H), 1.79-1.72 (m, 1H), 1.70-1.59 (m, 3H), 1.44 (tt, J=2.8, 13.6 Hz, 1H).

### Step 2: Synthesis of (3aS,6S,7aS)-3a-(3,4-dimethoxyphenyl)-1-methyloctahydro-1H-indol-6-yl dimethylcarbamate (101)

To a solution of 019 (100 mg, 343 umol) in THF (2 mL) was added t-BuOK (1 M in THF, 686 uL) and N,N-dimethylcarbamoyl chloride (73.8 mg, 686 umol, 63.0 uL). The reaction mixture was allowed to stir at 25° C. for 1 hr and then concentrated in vacuo. The resulting residue was purified by prep-HPLC (column: Phenomenex luna C_{18 150*25} mm*10 um; mobile phase: [water (FA)-ACN]; B %: 5%-35%, 8 min) to give 101 (419) (86.7 mg, 72%) as white oil. LC-MS (ESI⁺) m/z 363.2 (M+H)⁺. ¹H NMR (400 MHZ, CDCl₃) δ 6.94-6.89 (m, 1H), 6.88-6.81 (m, 2H), 4.76 (td, J=3.6, 7.2 Hz, 1H), 3.89 (d, J=10.8 Hz, 6H), 3.52-3.41 (m, 1H), 3.30 (t, J=6.8 Hz, 1H), 2.93 (s, 6H), 2.68-2.58 (m, 1H), 2.42 (s, 3H), 2.30-2.11 (m, 4H), 2.05-1.94 (m, 1H), 1.91-1.81 (m, 1H), 1.75-1.62 (m, 2H).

### Example 4: Synthesis of (3aS,6S,7aS)-3a-(3,4-dimethoxyphenyl)-1-methyloctahydro-1H-indol-6-yl pivalate (102)

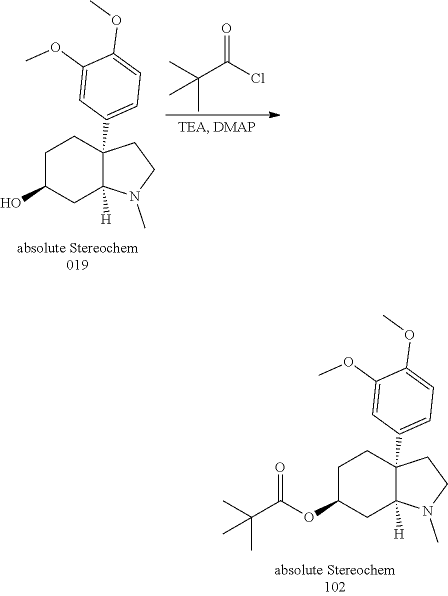

To a solution of (3aS,6S,7aS)-3a-(3,4-dimethoxyphenyl)-1-methyloctahydro-1H-indol-6-ol (019) (100 mg, 343 umol) and 2,2-dimethylpropanoyl chloride (82.7 mg, 686 umol, 84.4 uL) in DCM (1.0 mL) was added TEA (69.4 mg, 686 umol, 95.5 uL) and DMAP (4.19 mg, 34.32 umol). The reaction mixture was allowed to stir at 25° C. for 2 hr and then concentrated. The residue was purified by prep-HPLC (column: Waters xbridge 150*25 mm 10 um; mobile phase: [water (NH₄HCO₃)-ACN]; B %: 54%-84%, 11 min) to give 102 (90.99 mg, 88%) as a brown gum. LC-MS (ESI⁺) m/z 376.2 (M+H)⁺. ¹H NMR (400 MHZ, CDCl₃) δ 6.96-6.87 (m, 2H), 6.87-6.78 (m, 1H), 4.84 (t, J=4.4 Hz, 1H), 3.90 (d, J=9.2 Hz, 6H), 3.17-3.00 (m, 1H), 2.84-2.66 (m, 1H), 2.42-2.21 (m, 5H), 2.14-1.98 (m, 2H), 1.96-1.73 (m, 3H), 1.70-1.61 (m, 2H), 1.23 (s, 9H).

### Example 5: Synthesis of (3aS,6S,7aS)-3a-(3,4-dimethoxyphenyl)-1-methyloctahydro-1H-indol-6-yl isobutyrate (103)

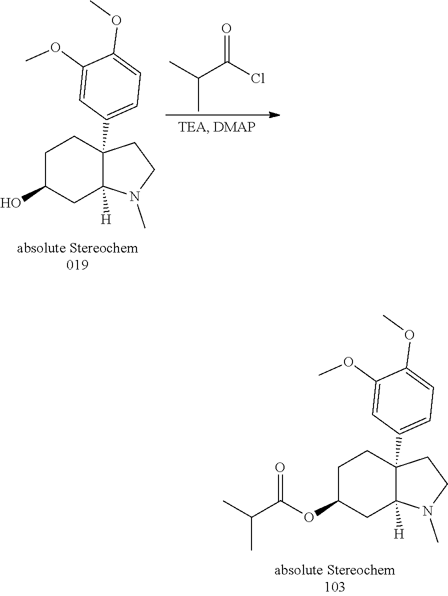

To a solution of (3aS,6S,7aS)-3a-(3,4-dimethoxyphenyl)-1-methyloctahydro-1H-indol-6-ol (019) (100 mg, 343 umol) in DCM (1.0 mL) was added DMAP (4.19 mg, 34.3 umol), TEA (104 mg, 1.03 mmol, 143 uL) and 2-methylpropanoyl chloride (73.1 mg, 686 umol, 71.7 uL). The reaction mixture was allowed to stir at 25° C. for 2 hr and then concentrated in vacuo. The residue was purified by prep-HPLC (column: Welch Xtimate C18 150*25 mm*5 um; mobile phase: [water(NH3H2O)-ACN]; B %: 50%-80%, 8 min) to give 103 (83.6 mg, 81%) as a yellow gum. LC-MS (ESI⁺) m/z 362.1 (M+H)⁺. ¹H NMR (400 MHZ, CDCl₃) δ 6.97-6.86 (m, 2H), 6.85-6.77 (m, 1H), 4.86 (t, J=4.6 Hz, 1H), 3.89 (d, J=9.2 Hz, 6H), 3.06 (br s, 1H), 2.76 (br s, 1H), 2.64-2.45 (m, 1H), 2.42-2.17 (m, 5H), 2.13-1.97 (m, 2H), 1.95-1.84 (m, 2H), 1.82-1.74 (m, 1H), 1.69-1.63 (m, 1H), 1.56-1.49 (m, 1H), 1.28-0.99 (m, 6H).

### Example 6: Synthesis of (3aS,6S,7aS)-3a-(3,4-dimethoxyphenyl)-1-methyloctahydro-1H-indol-6-yl benzoate (104)

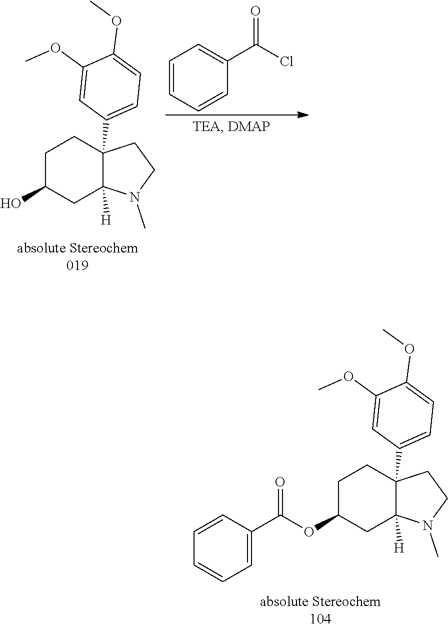

A mixture of (3aS,6S,7aS)-3a-(3,4-dimethoxyphenyl)-1-methyloctahydro-1H-indol-6-ol (019) (100 mg, 343 umol), benzoyl chloride (57.9 mg, 411 umol, 47.8 uL), and TEA (52.1 mg, 514 umol, 71.6 uL) in DCM (3.00 mL) was degassed and purged with N₂ 3 times, and then the reaction mixture was allowed to stir at 25° C. for 5 hr under an atmosphere of N₂. The reaction mixture was concentrated in vacuo and the residue was purified by prep-HPLC (FA condition: column: Phenomenex luna C18 150*25 mm*10 um; mobile phase: [water (FA)-ACN]; B %: 15%-45%, 8 min) to give 104 (61.70 mg, 45%) as white gum. LC-MS (ESI⁺) m/z 396.1 (M+H)⁺. ¹H NMR (400 MHZ, CDCl₃) δ 8.14-8.03 (m, 2H), 7.62-7.54 (m, 1H), 7.51-7.42 (m, 2H), 6.98-6.93 (m, 1H), 6.91-6.86 (m, 2H), 5.17-5.02 (m, 1H), 3.92 (d, J=15.6 Hz, 6H), 3.84-3.71 (m, 2H), 2.84 (dt, J=8.4, 10.6 Hz, 2H), 2.55 (s, 3H), 2.44-2.36 (m, 2H), 2.30-2.23 (m, 2H), 2.02-1.89 (m, 2H), 1.81 (ddd, J=4.4, 8.8, 13.0 Hz, 1H).

### Example 7: Synthesis of (3aS,6S,7aS)-3a-(3,4-dimethoxyphenyl)-1-methyloctahydro-1H-indol-6-yl pentanoate (105)

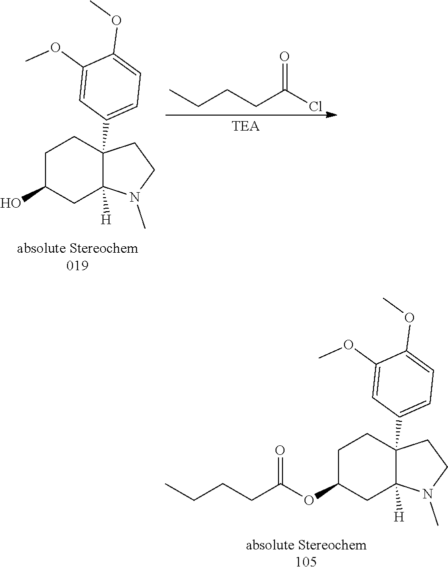

To a solution of a solution of (3aS,6S,7aS)-3a-(3,4-dimethoxyphenyl)-1-methyloctahydro-1H-indol-6-ol (019) (100 mg, 343 umol) and pentanoyl chloride (62.0 mg, 515 umol, 62.4 ul) in DCM (3 mL) was added TEA (52.1 mg, 515 umol, 71.7 uL). The reaction mixture was allowed to stir at 25° C. for 3 hr and then concentrated in vacuo. The residue was purified by prep-HPLC (column: Phenomenex luna C18 150*25 mm*10 um; mobile phase: [water (FA)-ACN]; B %: 11%-41%, 10 min) to give 105 (57.4 mg, 31%) as a yellow gum. LC-MS (ESI⁺) m/z. 376.3 (M+H)⁺. ¹H NMR (400 MHZ, CDCl₃) δ 6.93-6.87 (m, 2H), 6.84-6.80 (m, 1H), 4.87 (m, 1H), 3.88 (d, J=9.2 Hz, 6H), 3.08 (t, J=7.6 Hz, 1H), 2.84-2.75 (m, 1H), 2.39-2.28 (m, 6H), 2.24-2.17 (m, 1H), 2.08-1.94 (m, 3H), 1.93-1.87 (m, 2H), 1.82-1.77 (m, 1H), 1.68-1.62 (m, 2H), 1.62-1.51 (m, 2H), 1.38 (m, 2H), 0.93 (t, J=7.2 Hz, 3H).

### Example 8: Synthesis of (3aS,6S,7aS)-3a-(3,4-dimethoxyphenyl)-1-methyloctahydro-1H-indol-6-yl butyrate (106)

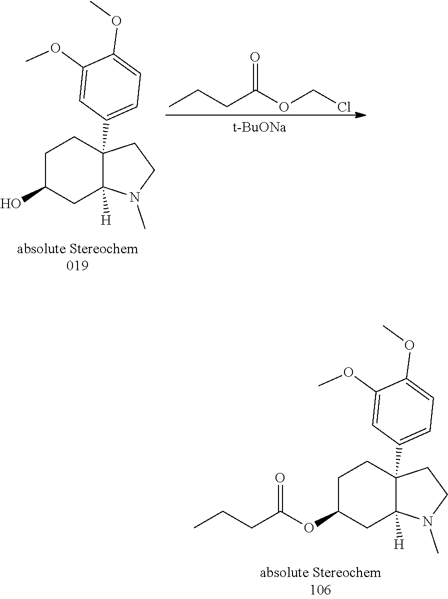

A mixture of (3aS,6S,7aS)-3a-(3,4-dimethoxyphenyl)-1-methyloctahydro-1H-indol-6-ol (019) (400 mg, 1.37 mmol), chloromethyl butanoate (375 mg, 2.75 mmol) and t-BuONa (264 mg, 2.75 mmol) in THF (5 mL) was degassed and purged with N₂ 3 times. The mixture was allowed to stir at 25° C. for 2 hr under and atmosphere of N₂. The reaction mixture was filtered and the filtrate was concentrated under reduced pressure. The residue was purified by prep-HPLC (FA condition: column: Phenomenex luna C18 150*25 mm*10 um; mobile phase: [water (FA)-ACN]; B %: 17%-37%, 58 min) to give 106 (169 mg, 34%) as a grey oil. LC-MS (ESI⁺) m/z 362.3 (M+H)⁺1H NMR (400 MHZ, CDCl₃) δ=6.93-6.81 (m, 3H), 4.91-4.84 (m, 1H), 3.89 (d, J=10.0 Hz, 6H), 3.47-3.37 (m, 1H), 3.22 (t, J=6.4 Hz, 1H), 2.60 (q, J=9.2 Hz, 1H), 2.42 (s, 3H), 2.32 (t, J=7.2 Hz, 2H), 2.25-2.09 (m, 4H), 2.00 (td, J=7.2, 14.4 Hz, 1H), 1.91-1.82 (m, 1H), 1.72-1.60 (m, 4H), 0.96 (t, J=7.2 Hz, 3H).

### Example 9: Synthesis of (((3aS,6S,7aS)-3a-(3,4-dimethoxyphenyl)-1-methyloctahydro-1H-indol-6-yl)oxy)methyl pivalate (107)

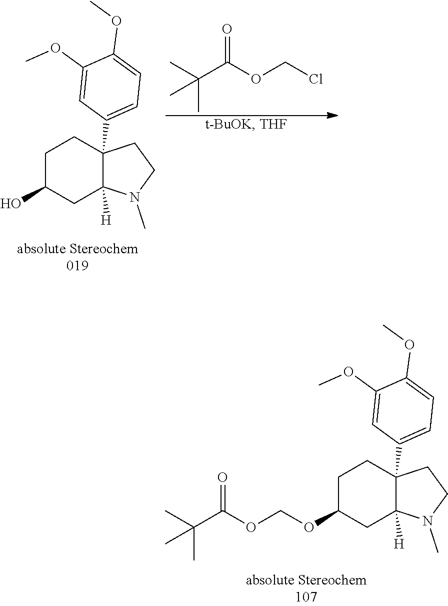

To a solution of (3aS,6S,7aS)-3a-(3,4-dimethoxyphenyl)-1-methyloctahydro-1H-indol-6-ol (019) (120 mg, 411 umol) in THF (1.0 mL) was added t-BuOK (1M, 823 uL). The mixture was allowed to stir at 0° C. for 10 min and then chloromethyl 2,2-dimethylpropanoate (124 mg, 823 umol, 119 uL) was added. The reaction mixture was allowed to stir at 25° C. for 2 hr and then concentrated in vacuo. The residue was purified by prep-HPLC (column: Waters xbridge 150*25 mm 10 um; mobile phase: [water(NH₄HCO₃)-ACN]; B %: 28%-58%, 8 min) to give 107 (14.62 mg, 8%) as a yellow gum. LC-MS (ESI⁺) m/z 406.2 (M+H)⁺; 1H NMR (400 MHZ, CDCl₃) δ 6.87-6.78 (m, 2H), 6.77-6.70 (m, 1H), 5.35-5.24 (m, 2H), 3.81 (d, J=9.6 Hz, 6H), 3.73-3.60 (m, 1H), 3.17-3.00 (m, 1H), 2.69 (s, 1H), 2.33 (s, 3H), 2.18-2.08 (m, 1H), 2.03-1.78 (m, 5H), 1.76-1.68 (m, 2H), 1.48-1.37 (m, 1H), 1.14 (s, 9H).

### Example 10: Synthesis of (((3aS,6S,7aS)-3a-(3,4-dimethoxyphenyl)-1-methyloctahydro-1H-indol-6-yl)oxy)methyl isobutyrate (108)

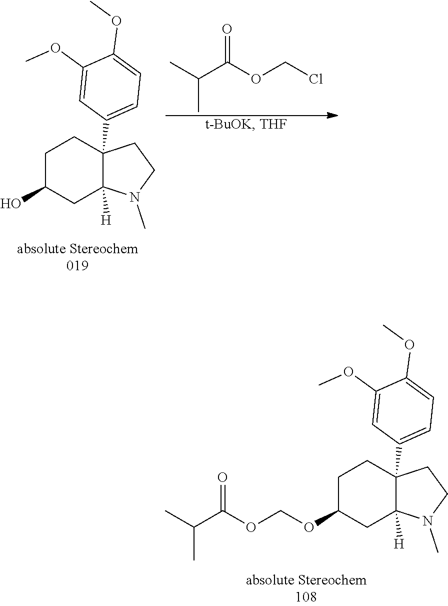

A mixture of chloromethyl 2-methylpropanoate (210 mg, 1.54 mmol), (3aS,6S,7aS)-3a-(3,4-dimethoxyphenyl)-1-methyloctahydro-1H-indol-6-ol (019) (150 mg, 514 umol) and t-BuOK (1 M in THF, 1.54 mL) in THF (1 mL) was degassed and purged with N₂ 3 times. The mixture was allowed to stir at 80° C. for 2 hr under an atmosphere of N₂. The reaction mixture was filtered and concentrated in vacuo. The residue was purified by prep-HPLC (column: Phenomenex luna C18 150*25 mm*10 um; mobile phase: [water (FA)-ACN]; B %: 12%-42%, 8 min) to give 108 (18.29 mg, 20%) as a white solid. LC-MS (ESI⁺) m/z 392.5 (M+H)⁺. ¹H NMR (400 MHz, CDCl₃): δ 6.74-7.01 (m, 3H), 4.73 (br dd, J=6.4, 4.8 Hz, 1H), 3.93-3.97 (m, 1H), 3.91 (s, 3H), 3.88 (s, 3H), 3.32-3.55 (m, 2H), 2.55-2.96 (m, 1H), 2.48 (s, 1H), 2.46 (s, 2H), 2.25-2.33 (m, 2H), 2.07-2.21 (m, 3H), 1.96-2.04 (m, 2H), 1.89-1.91 (m, 1H), 1.85-1.93 (m, 5H), 1.37-1.39 (m, 1H), 1.23-1.37 (m, 1H), 1.34 (s, 1H), 0.98 (s, 2H), 0.94-1.02 (m, 1H).

### Example 11: Synthesis of (3aS,6S,7aS)-3a-(3,4-dimethoxyphenyl)-1-methyloctahydro-1H-indol-6-yl ethyl carbonate (109)

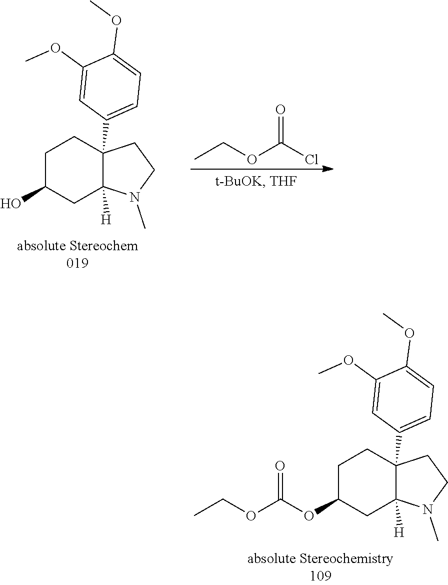

To a solution of (3aS,6S,7aS)-3a-(3,4-dimethoxyphenyl)-1-methyloctahydro-1H-indol-6-ol (019) (100 mg, 343 umol) in THF (1.0 mL) was added t-BuOK (1M, 686 uL). The mixture was allowed to stir at 0° C. for 30 min and then add ethyl carbonochloridate (74.5 mg, 686 umol, 65.3 uL) was added to the mixture at 0° C. The reaction mixture was stirred at 25° C. for 2 hr and then poured into aq.NaHCO₃ (50 mL). The mixture was extracted with ethyl acetate (50 mL). The organic solutions were combined, dried over sodium sulfate, filtered and concentrated in vacuo. The residue was purified by prep-HPLC (column: Phenomenex luna C18 150*25 mm*10 um; mobile phase: [water (FA)-ACN]; B %: 7%-37%, 8 min) to give 109 (17.7 mg, 17%) as an off-white gum. LC-MS (ESI⁺) m/z 364.3 (M+H)⁺. ¹H NMR (400 MHZ, CDCl₃) δ 6.94-6.85 (m, 2H), 6.85-6.81 (m, 1H), 4.78-4.70 (m, 1H), 4.29-4.11 (m, 2H), 3.89 (d, J=10.4 Hz, 6H), 3.44-3.38 (m, 1H), 3.26 (t, J=6.4 Hz, 1H), 2.70-2.59 (m, 1H), 2.43 (s, 3H), 2.28-2.02 (m, 5H), 1.93-1.82 (m, 1H), 1.79-1.66 (m, 2H), 1.33 (t, J=7.2 Hz, 3H).

### Example 12: Synthesis of (3aS,6R,7aS)-3a-(3,4-dimethoxyphenyl)-1-methyloctahydro-1H-indol-6-ol (018, (−)-mesembranol)

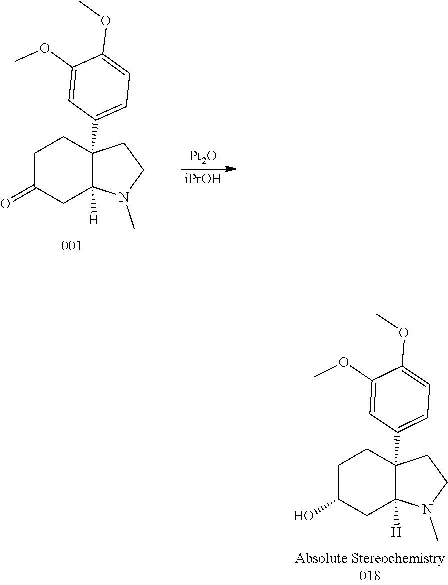

A mixture of 001 (200 mg, 691 umol) and PtO₂ (20.0 mg, 88.0 umol) in IPA (4 mL) was degassed and purged with N₂ 3 times. The mixture was allowed to stir at 25° C. for 16 hr under an atmosphere of N₂. The reaction mixture was filtered and concentrated under reduced pressure. The residue was purified by re-crystallization from EtOH (1 mL) at 25° C. to give (3aS,6R,7aS)-3a-(3,4-dimethoxyphenyl)-1-methyloctahydro-1H-indol-6-ol (018, (−)-mesembranol) (100 mg, 49%) as a white solid. LC-MS (ESI⁺) m/z 292.4 (M+H). 1H NMR (400 MHZ, CDCl₃) δ 6.86-6.78 (m, 2H), 6.77-6.71 (m, 1H), 3.86 (s, 1H), 3.81 (d, J=6.8 Hz, 6H), 3.30 (dt, J=6.8, 9.6 Hz, 1H), 2.83 (s, 1H), 2.40 (s, 3H), 2.33-2.20 (m, 1H), 2.09 (dd, J=2.8, 14.8 Hz, 1H), 1.90-1.82 (m, 2H), 1.78 (dd, J=6.8, 11.6 Hz, 1H), 1.67-1.62 (m, 2H), 1.57 (td, J=2.8, 14.8 Hz, 1H), 1.39-1.30 (m, 2H).

### Example 13: Synthesis of (((3aS,6S,7aS)-3a-(3,4-dimethoxyphenyl)-1-methyloctahydro-1H-indol-6-yl)oxy)methyl butyrate (110)

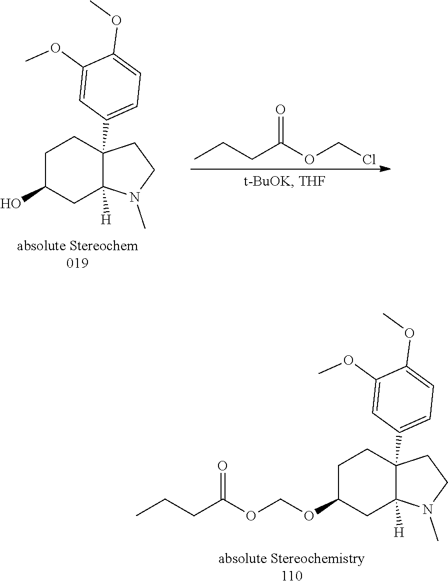

Compound 019 will be reacted with chloromethyl butyrate in the presence of a base such as tBuOK in a solvent such as THF to give 110.

### Example 14: Synthesis of (3aS,6R,7aS)-3a-(3,4-dimethoxyphenyl)-1-methyloctahydro-1H-indol-6-yl dimethylcarbamate (201)

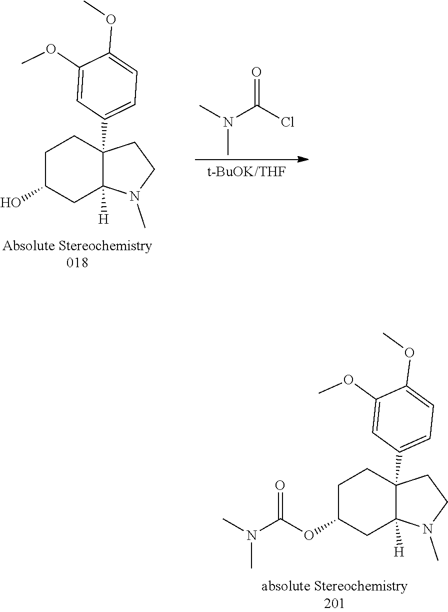

To a solution of 018 in THF (4 mL) at 0° C. was added t-BuOK (1M, 1.37 mL). The reaction mixture was allowed to stir at 0° C. for 0.5 hr and then N,N-dimethylcarbamoyl chloride (147 mg, 1.37 mmol) was added. The reaction mixture was allowed to stir at 25° C. for 2 hr and then concentrated in vacuo. The residue was purified by prep-HPLC (column: Phenomenex luna C18 150*25 mm*10 um; mobile phase: [water (FA)-ACN]; B %: 1%-31%, 10 min) to give 201 (108 mg, 54%) as a colorless gum.

### Example 15: Synthesis of (3aS,6R,7aS)-3a-(3,4-dimethoxyphenyl)-1-methyloctahydro-1H-indol-6-yl pivalate (202)

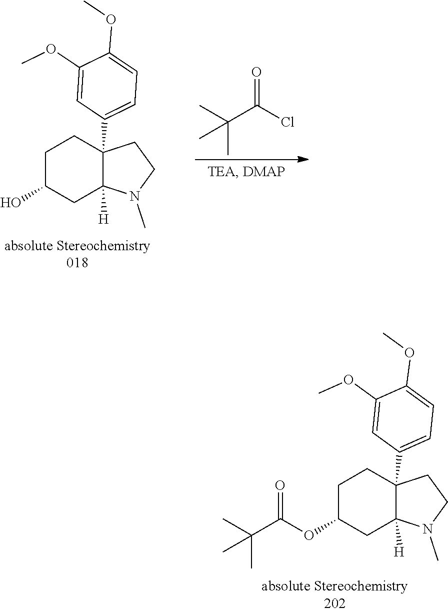

To a solution of 018 (100 mg, 343 umol) and 2,2-dimethylpropanoyl chloride (82.7 mg, 686 umol, 84 uL) in DCM (1.0 mL) was added TEA (69.4 mg, 686 umol, 95 uL) and DMAP (4.19 mg, 34.3 umol). The mixture was allowed to stir at 25° C. for 2 hr and then concentrated in vacuo. The residue was purified by prep-HPLC (column: Waters xbridge 150*25 mm 10 um; mobile phase: [water(NH₄HCO₃)-ACN]; B %: 48%-78%, 8 min) to give 202 (86.44 mg, 83%) as an off-white solid. LC-MS (ESI⁺) m/z 376.3 (M+H)⁺. ¹H NMR (400 MHZ, CDCl₃) δ 6.90-6.73 (m, 3H), 5.00 (s, 1H), 3.82 (d, J=6.0 Hz, 6H), 3.39-3.10 (m, 1H), 2.95-2.62 (m, 1H), 2.34 (s, 4H), 2.18-1.98 (m, 3H), 1.92-1.67 (m, 3H), 1.31-1.16 (m, 2H), 1.07 (s, 9H).

### Example 16: Synthesis of (3aS,6R,7aS)-3a-(3,4-dimethoxyphenyl)-1-methyloctahydro-1H-indol-6-yl isobutyrate (203)

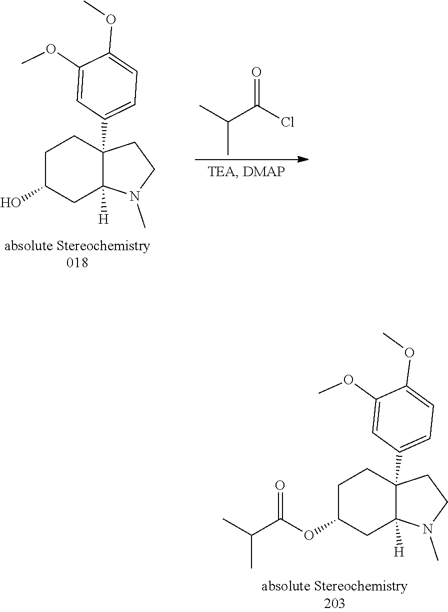

To a solution of 018 (10 mg, 343 umol) and 2-methylpropanoyl chloride (73.1 mg, 686 umol, 71 uL) in DCM (1.0 mL) was added TEA (69.4 mg, 686 umol, 95 uL) and DMAP (4.19 mg, 34.3 umol). The mixture was allowed to stir at 25° C. for 2 hr and then concentrated in vacuo. The residue was purified by prep-HPLC (column: Waters xbridge 150*25 mm 10 um; mobile phase: [water(NH₄HCO₃)-ACN]; B %: 42%-72%, 8 min) to give 203 (71.8 mg, 71%) as a yellow gum. LC-MS (ESI⁺) m/z 362.4 (M+H)⁺. ¹H NMR (400 MHZ, CDCl₃) δ=6.91-6.70 (m, 3H), 5.07-4.87 (m, 1H), 3.82 (d, J=6.8 Hz, 6H), 3.16 (d, J=1.2 Hz, 1H), 2.71 (d, J=2.4 Hz, 1H), 2.37 (td, J=7.2, 14.0 Hz, 1H), 2.33-2.18 (m, 4H), 2.16-1.93 (m, 4H), 1.89-1.79 (m, 1H), 1.77-1.67 (m, 2H), 1.25-1.19 (m, 1H), 1.04 (dd, J=3.2, 6.8 Hz, 6H).

### Example 17: Synthesis of (3aS,6R,7aS)-3a-(3,4-dimethoxyphenyl)-1-methyloctahydro-1H-indol-6-yl benzoate (204)

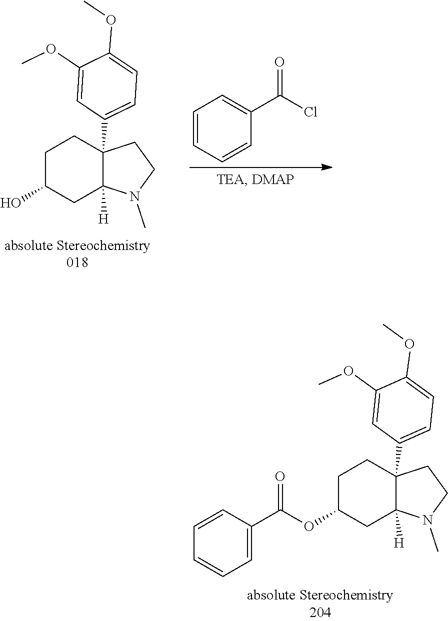

To a solution of 018 (100 mg, 343 umol) and DMAP (4.19 mg, 34.3 umol) in DCM (1.0 mL) was added TEA (104 mg, 1.03 mmol, 143 uL) and benzoyl chloride (57.9 mg, 412 umol, 47.8 uL). The mixture was allowed to stir at 25° C. for 1 hr and then concentrated in vacuo. The residue was purified by prep-HPLC (column: Phenomenex luna C18 150*25 mm*10 um; mobile phase: [water (FA)-ACN]; B %: 12%-42%, 8 min) to give 204 (73.2 mg, 72%) as a yellow gum. LC-MS (ESI⁺) m/z 396.2 (M+H)⁺. ¹H NMR (400 MHZ, CDCl₃) δ 8.04-7.95 (m, 2H), 7.58-7.51 (m, 1H), 7.46-7.37 (m, 2H), 6.97-6.91 (m, 1H), 6.90-6.83 (m, 2H), 5.56-5.40 (m, 1H), 3.90 (d, J=3.2 Hz, 6H), 3.81-3.70 (m, 1H), 3.38 (t, J=4.0 Hz, 1H), 2.75-2.62 (m, 4H), 2.52-2.40 (m, 1H), 2.27-2.14 (m, 3H), 2.14-2.07 (m, 1H), 2.07-1.95 (m, 2H), 1.64-1.50 (m, 1H)

### Example 18: Synthesis of (3aS,6S,7aS)-3a-(3,4-dimethoxyphenyl)-1-methyloctahydro-1H-indol-6-yl pentanoate (205)

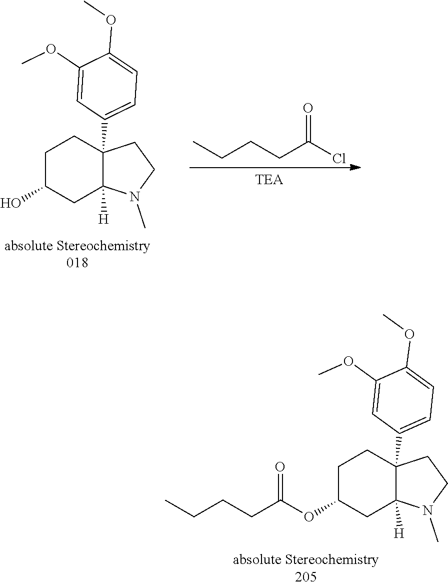

To a solution of 018 and DMAP (4.19 mg, 34.3 umol) in DCM (1.0 mL) was added TEA (104 mg, 1.03 mmol, 143 uL) and pentanoyl chloride (49.6 mg, 412 umol, 49.9 uL). The reaction mixture was allowed to stir at 25° C. for 1 hr and then concentrated in vacuo. The residue was purified by prep-HPLC (column: Phenomenex luna C18 150*25 mm*10 um; mobile phase: [water (FA)-ACN]; B %: 10%-40%, 10 min) to give 205 (77.6 mg, 78%) as a colorless oil. LC-MS (ESI⁺) m/z 376.2 (M+H)⁺. ¹H NMR (400 MHZ, CDCl₃) δ 6.94-6.80 (m, 3H), 5.23-5.16 (m, 1H), 3.90 (d, J=7.0 Hz, 6H), 3.77-3.67 (m, 1H), 3.28 (t, J=3.8 Hz, 1H), 2.71-2.55 (m, 4H), 2.38-2.27 (m, 1H), 2.24 (t, J=7.6 Hz, 2H), 2.19-2.09 (m, 3H), 2.08-1.99 (m, 1H), 1.90-1.78 (m, 2H), 1.64-1.53 (m, 2H), 1.43-1.26 (m, 3H), 0.89 (t, J=7.2 Hz, 3H).

### Example 19: Synthesis of (3aS,6S,7aS)-3a-(3,4-dimethoxyphenyl)-1-methyloctahydro-1H-indol-6-yl butyrate (206)

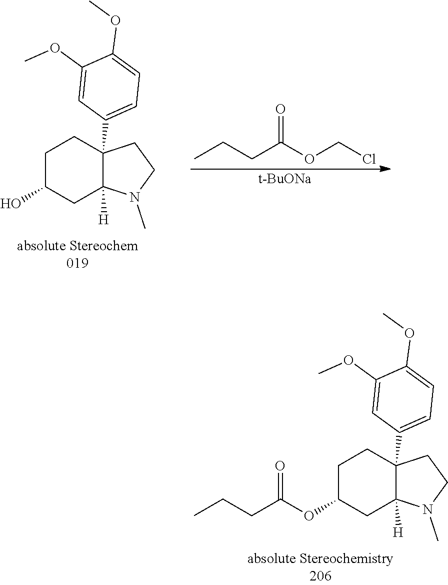

To a solution of butanoylbutanoate (108 mg, 686 umol, 112 uL) in DCM (2 mL) was added TEA (69.4 mg, 686 umol, 95.5 uL), 018 (100 mg, 343 umol) and DMAP (4.19 mg, 34.3 umol). The reaction mixture was allowed to stir at 25° C. for 2 hr and then concentrated in vacuo. The residue was purified by prep-HPLC (column: Phenomenex C_{18 150*25} mm*10 um; mobile phase: [water(NH₄HCO₃)-ACN]; B %: 39%-79%, 14 min) to give 206 (47.7 mg, 48%) as a grey solid. LC-MS (ESI⁺) m/z 362.2 (M+H)⁺; ¹H NMR (400 MHZ, CDCl₃) δ=6.96-6.79 (m, 3H), 5.16-5.00 (m, 1H), 4.02-3.83 (m, 6H), 3.31-3.15 (m, 1H), 2.86-2.69 (m, 1H), 2.42-2.35 (m, 3H), 2.31 (br dd, J=5.4, 6.4 Hz, 1H), 2.25-2.16 (m, 3H), 2.16-2.02 (m, 2H), 1.99-1.87 (m, 1H), 1.84-1.76 (m, 2H), 1.82-1.75 (m, 1H), 1.68-1.60 (m, 3H), 1.66-1.55 (m, 7H), 1.39-1.22 (m, 2H), 1.00-0.83 (m, 3H).

### Example 20: Synthesis of (((3aS,6R,7aS)-3a-(3,4-dimethoxyphenyl)-1-methyloctahydro-1H-indol-6-yl)oxy)methyl pivalate (207)

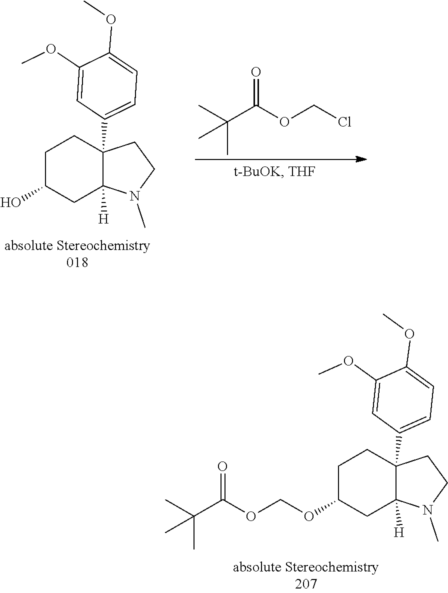

To a solution of 018 (150 mg, 515 umol) in THF (4 mL) was added t-BuOK (1M in THF, 1.03 mL). To the reaction mixture was added chloromethyl 2,2-dimethylpropanoate (310 mg, 2.06 mmol, 298 uL). The reaction mixture was allowed to stir at 25° C. for 2 hr and then filtered. The eluant was concentrated in vacuo. The residue was purified by prep-HPLC (column: Phenomenex luna C18 150*25 mm*10 um; mobile phase: [water (FA)-ACN]; B %: 10%-40%, 8 min) to give 207 (55 mg, 28%) as a colourless oil. LC-MS (ESI⁺) m/z 406.6 (M+H)⁺. ¹H NMR (400 MHZ, CDCl₃) δ=6.89-6.76 (m, 3H), 5.39-5.32 (m, 1H), 5.28 (d, J=6.4 Hz, 1H), 4.24-4.05 (m, 1H), 3.89 (d, J=6.8 Hz, 6H), 3.85-3.76 (m, 1H), 3.46-3.14 (m, 1H), 2.63 (s, 4H), 2.27 (s, 1H), 2.21-2.00 (m, 4H), 1.96-1.80 (m, 2H), 1.39-1.27 (m, 1H), 1.21 (s, 9H).

### Example 21: Synthesis of (((3aS,6R,7aS)-3a-(3,4-dimethoxyphenyl)-1-methyloctahydro-1H-indol-6-yl)oxy)methyl isobutyrate (208)

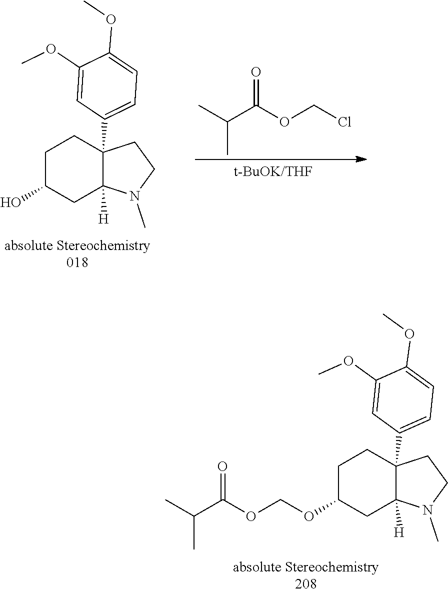

To a solution of 018 (150 mg, 514 umol) in THF (4 mL) was added t-BuOK (1M in THF, 1.03 mL). To the reaction mixture was added chloromethyl 2-methylpropanoate (281 mg, 2.06 mmol). The reaction mixture was allowed to stir at 25° C. for 2 hr and then filtered. The eluant was concentrated in vacuo. The residue was purified by prep-HPLC (column: Waters xbridge 150*25 mm 10 um; mobile phase: [water(NH₄HCO₃)-ACN]; B %: 42%-72%, 8 min) to give 208 (15 mg, 8%) as a yellow gum. LC-MS (ESI⁺) m/z 392.1 (M+H)⁺. ¹H NMR (400 MHZ, CDCl₃) δ=6.88-6.67 (m, 3H), 5.40-5.14 (m, 2H), 3.81 (d, J=6.4 Hz, 7H), 3.33-3.02 (m, 1H), 2.67 (d, J=4.0 Hz, 1H), 2.53-2.43 (m, 1H), 2.42-2.05 (m, 5H), 1.99 (s, 2H), 1.92-1.67 (m, 3H), 1.35-1.14 (m, 2H), 1.12 (d, J=1.6 Hz, 3H), 1.11-1.08 (m, 3H)

### Example 22: Synthesis of (3aS,6R,7aS)-3a-(3,4-dimethoxyphenyl)-1-methyloctahydro-1H-indol-6-yl ethyl carbonate (209)

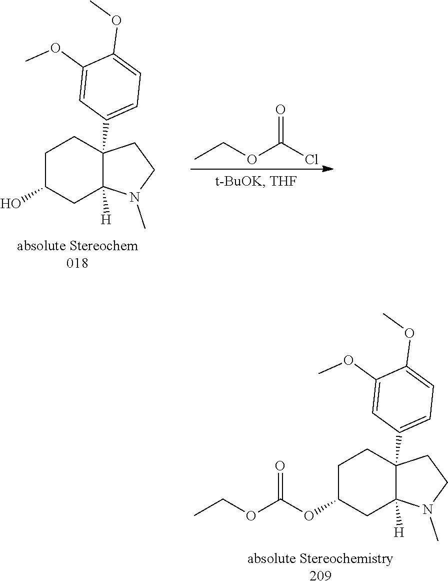

To a solution of 018 (150 mg, 514 umol) in THF (2 mL) at 0° C. was added t-BuOK (1M, 1.03 mL) over 0.5 h, then ethyl carbonochloridate (111 mg, 1.03 mmol, 98.0 uL) was added. The reaction mixture was allowed to stir at 25° C. for 2 hr and then filtered. The filtrate was concentrated in vacuo. The residue was purified by prep-HPLC (column: Phenomenex luna C18 150*25 mm*10 um; mobile phase: [water (FA)-ACN]; B %: 5%-35%, 8 min) to give 209 (27.6 mg, 18%) as gray gum. LC-MS (ESI⁺) m/z 364.0 (M+H)⁺; 1H NMR (400 MHZ, CDCl₃) δ 7.14-6.70 (m, 3H), 4.13-3.99 (m, 1H), 3.98-3.83 (m, 6H), 3.24 (dt, J=4.4, 9.2 Hz, 1H), 2.77 (br s, 1H), 2.39 (s, 2H), 2.30-2.28 (m, 1H), 2.27-2.17 (m, 1H), 2.13-2.04 (m, 2H), 1.99-1.89 (m, 1H), 1.88-1.76 (m, 2H), 1.74-1.67 (m, 1H), 1.58-1.51 (m, 1H), 1.20 (br s, 1H), 1.43-1.15 (m, 1H).

### Example 23: Synthesis of (((3aS,6R,7aS)-3a-(3,4-dimethoxyphenyl)-1-methyloctahydro-1H-indol-6-yl)oxy)methyl butyrate (210)

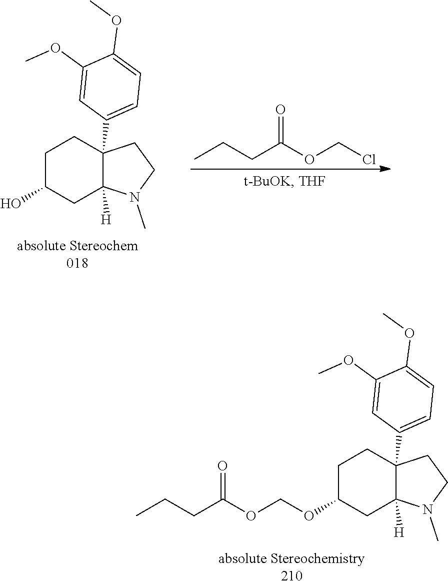

To a solution of 018 (150 mg, 515 umol) in THF (4 mL) was added t-BuOK (1 M in THF, 1.03 mL). To the reaction mixture was added chloromethyl butanoate (281 mg, 2.06 mmol). The reaction mixture was allowed to stir at 25° C. for 2 hr and the filtered. The eluant was concentrated in vacuo to give a residue that was purified by prep-HPLC (column: Waters xbridge 150*25 mm 10 um; mobile phase: [water(NH₄HCO₃)-ACN]; B %: 42%-72%, 8 min) to give 210 (18.0 mg, 10%) as a yellow gum. LC-MS (ESI⁺) m/z 392.1 (M+H)⁺. ¹H NMR (400 MHZ, CDCl₃-d) δ=7.02-6.60 (m, 3H), 5.46-5.19 (m, 2H), 3.89 (d, J=6.0 Hz, 7H), 3.20 (d, J=9.2 Hz, 1H), 2.91-2.66 (m, 1H), 2.54-2.25 (m, 6H), 2.24-2.13 (m, 1H), 2.10-2.00 (m, 2H), 1.98-1.75 (m, 3H), 1.74-1.62 (m, 3H), 1.26 (s, 1H), 0.96 (t, J=7.4 Hz, 3H).

### Example 24: Synthesis of (3aS,6R,7aS)-3a-(3,4-dimethoxyphenyl)-1-methyloctahydro-1H-indol-6-yl dihydrogen phosphate (211)

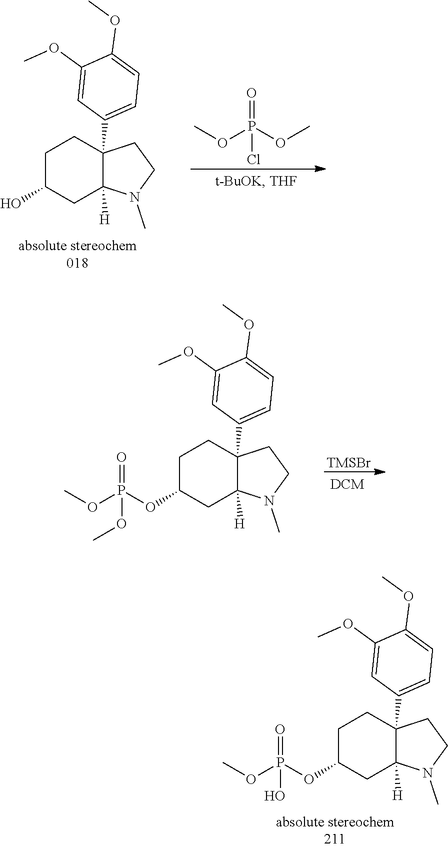

### Step 1: Synthesis of (3aS,6R,7aS)-3a-(3,4-dimethoxyphenyl)-1-methyloctahydro-1H-indol-6-yl dimethyl phosphate

To a solution of 018 (100 mg, 343 umol) in THF (2.0 mL) was added t-BuOK (1M in THF, 686 uL) at 0° C. The reaction mixture was allowed to stir at 0° C. for 10 min and then [chloro(methoxy)phosphoryl]oxymethane (148 mg, 1.03 mmol, 111 uL) was added. The reaction mixture was allowed to stir at 25° C. for 2 hr and then filtered. The filtrate was concentrated in vacuo to give the title compound (110 mg, 67%) as a brown oil. LC-MS (ESI⁺) m/z 400.2 (M+H)⁺.

### Step 2: Synthesis of (3aS,6R,7aS)-3a-(3,4-dimethoxyphenyl)-1-methyloctahydro-1H-indol-6-yl dihydrogen phosphate (211)

To a solution of (3aS,6R,7aS)-3a-(3,4-dimethoxyphenyl)-1-methyloctahydro-1H-indol-6-yl dimethyl phosphate (100 mg, 250 umol) in DCM (1.0 mL) at 0° C. was added TMSBr (114 mg, 751 umol, 97 uL). The reaction mixture was allowed to stir at 25° C. for 16 hr and then filtered. The filtrate was concentrated in vacuo. The residue was purified by prep-HPLC (column: Waters xbridge 150*25 mm 10 um; mobile phase: [water(NH₃H₂O)-ACN]; B %: 0%-28%, 10 min) to give 211 (9.55 mg, 9%) as a white solid. LC-MS (ESI⁺) m/z 372.2 (M+H)⁺; 1H NMR (400 MHZ, CDCl₃) δ 6.92-6.66 (m, 3H), 4.80-4.49 (m, 2H), 3.90-3.77 (m, 6H), 3.62 (s, 1H), 3.08-2.73 (m, 4H), 2.71-2.54 (m, 1H), 2.25-1.64 (m, 6H), 1.37-1.05 (m, 1H).

### Example 25: Synthesis of (3aS,6S,7aS)-3a-(3,4-dimethoxyphenyl)-1-methyloctahydro-1H-indol-6-yl dihydrogen phosphate (111)

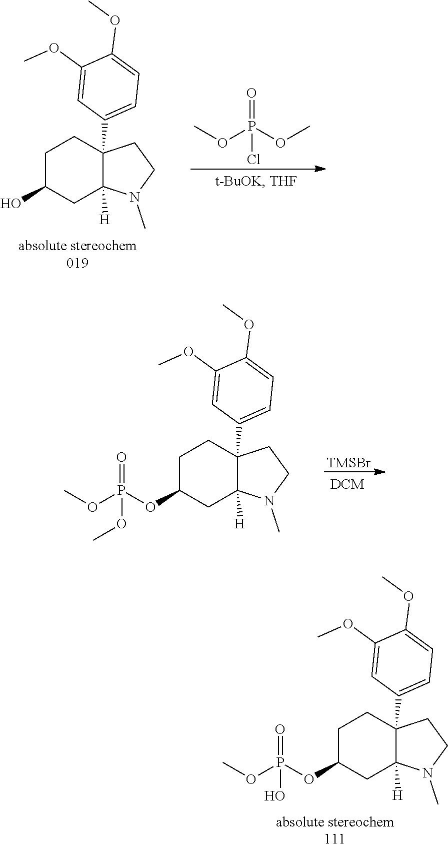

### Step 1: Synthesis of (3aS,6S,7aS)-3a-(3,4-dimethoxyphenyl)-1-methyloctahydro-1H-indol-6-yl dimethyl phosphate

To a solution of 019 (300 mg, 1.03 mmol) in THF (6 mL) at 0° C. was added t-BuOK (1M, 2.06 mL) and then [chloro(methoxy)phosphoryl]oxymethane (297 mg, 2.06 mmol). The reaction mixture was allowed to stir at 25° C. for 4 hr and then concentrated to give the title compound (300 mg, 45%, 62% purity) as a white solid.

### Step 2: Synthesis of (3aS,6S,7aS)-3a-(3,4-dimethoxyphenyl)-1-methyloctahydro-1H-indol-6-yl dihydrogen phosphate (111)

To a solution of (3aS,6S,7aS)-3a-(3,4-dimethoxyphenyl)-1-methyloctahydro-1H-indol-6-yl dimethyl phosphate (300 mg, 751 μmol) in DCM (5 mL) at 0° C. was added TMSBr (459 mg, 3.00 mmol, 389 μL). The reaction mixture was allowed to stir at 25° C. for 16 hr and then concentrated in vacuo. The residue was purified by prep-HPLC (column: Waters xbridge 150*25 mm 10 um; mobile phase: [water(NH₄HCO₃)-ACN]; B %: 1%-25%, 10 min) to give 111 (40.5 mg, 20%) as a white solid. LC-MS (ESI⁺) m/z 372.2 (M+H)⁺; ¹H NMR (400 MHZ, CD₃OD) δ=7.05-6.93 (m, 3H), 4.50-4.42 (m, 1H), 4.03-3.93 (m, 1H), 3.92-3.82 (m, 7H), 3.30-3.21 (m, 1H), 3.13-3.03 (m, 3H), 2.71-2.61 (m, 1H), 2.33-2.08 (m, 4H), 2.03-1.87 (m, 2H), 1.47-1.35 (m, 1H).

### Assay Procedures

### Example A1: Plasma Stability Assay

Compound stability in rat and human plasma was measured by incubating compound with rat or human plasma at 37° C. in a microwell plate for sequential time points, monitoring pro-drug disappearance and metabolite appearance (mesembranol or 6-epi-mesembranol). A 2 mM stock solution of test compounds were prepared by dissolving appropriate amount of compounds in DMSO. The 2 mM stock was further diluted 200-folds in rat or human plasma to attain a final concentration of 10 μM respectively (0.5% DMSO). 50 μL aliquots of positive controls and test compounds spiked into rat or human plasma (n=2) were added to a pre-warmed plate (37° C.) and shaken at 50 rpm. At each respective time point, the reaction was stopped by adding 500 μL of acetonitrile containing internal standards (100 nM aprozolam, 200 nM caffeine, 100 nM tolbutamide). All samples were vortexed for 10 minutes, followed by centrifugation at 3,220 g for 30 minutes to precipitate proteins. 100 μL of the supernatant is transferred to a new plate. The supernatant will be diluted with ultrapure water according to the LC-MS signal response and peak shape. The concentrations of test compounds and positive control were quantified in the test samples using LC-MS/MS. Results reported as % of prodrug compound remaining at time point, and % of metabolite (mesembranol or 6-epi-mesembranol,) accumulated at time point. The data is presented in Table 1.

TABLE 1

| | | rat plasma | | human plasma | |
|---|---|---|---|---|---|
| | Time | Prodrug | mesembranol/6-epi- | Prodrug | mesembranol/6-epi- |
| Compound | (min) | (%) | mesembranol (%) | (%) | mesembranol (%) |
| 102 | 0 | 100.0 | 3.2 | 100.00 | 3.4 |
| | 10 | >99.0 | 13.2 | 97.52 | 3.5 |
| | 30 | 77.8 | 27.0 | 95.20 | 4.0 |
| | 60 | 68.1 | 50.1 | 92.88 | 4.6 |
| | 120 | 42.3 | 69.6 | >99.0 | 5.8 |
| | 240 | 15.4 | 91.4 | 94.80 | 7.7 |
| 103 | 0 | 100.0 | 5.3 | 100.00 | 2.2 |
| | 10 | 56.1 | 54.0 | >99.0 | 3.3 |
| | 30 | 12.2 | >99.0 | >99.0 | 5.6 |
| | 60 | 0.6 | >99.0 | 97.28 | 9.2 |
| | 120 | 0.1 | >99.0 | 97.48 | 15.4 |
| | 240 | 0.0 | >99.0 | 87.17 | 27.8 |
| 104 | 0 | 100.0 | 5.2 | 100.00 | 0.4 |
| | 10 | 62.0 | 59.1 | >99.0 | 0.8 |
| | 30 | 27.5 | >99.0 | 97.34 | 1.5 |
| | 60 | 7.5 | >99.0 | 97.40 | 1.9 |
| | 120 | 1.4 | >99.0 | 95.15 | 3.2 |
| | 240 | 0.6 | >99.0 | 90.28 | 5.3 |
| 105 | 0 | 100.0 | 19.1 | 100.00 | 2.2 |
| | 10 | 10.8 | >99.0 | 96.40 | 11.1 |
| | 30 | 0.5 | >99.0 | 89.85 | 23.7 |
| | 60 | 0.1 | >99.0 | 81.61 | 39.8 |
| | 120 | 0.0 | >99.0 | 72.68 | 63.0 |
| | 240 | 0.0 | >99.0 | 50.16 | 86.1 |
| 101 | 0 | 100.0 | 0.0 | 100.00 | 0.0 |
| | 10 | 88.7 | 0.0 | >99.0 | 0.0 |
| | 30 | 95.0 | 0.0 | >99.0 | 0.0 |
| | 60 | 98.2 | 0.0 | >99.0 | 0.0 |
| | 120 | 99.4 | 0.0 | >99.0 | 0.0 |
| | 240 | 92.2 | 0.0 | >99.0 | 0.0 |
| 106 | 0 | 100.0 | 11.3 | 100.00 | 4.3 |
| | 10 | 39.9 | 89.0 | 93.04 | 11.7 |
| | 30 | 2.75 | >99.0 | 87.33 | 22.0 |
| | 60 | 0.7 | >99.0 | 79.39 | 34.0 |
| | 120 | 0.2 | >99.0 | 67.04 | 50.2 |
| | 240 | 0.1 | >99.0 | 50.22 | 76.6 |
| 202 | 0 | 100.0 | 2.2 | 100.0 | 0.0 |
| | 10 | 80.6 | 44.4 | >99.0 | 1.1 |
| | 30 | 38.9 | 86.7 | >99.0 | 2.6 |
| | 60 | 9.6 | >99.0 | 96.3 | 4.2 |
| | 120 | 0.6 | >99.0 | 94.7 | 6.3 |
| | 240 | 0.0 | >99.0 | 88.1 | 9.4 |
| 203 | 0 | 100.0 | 17.8 | 100.0 | 0.1 |
| | 10 | 0.1 | >99.0 | 0.1 | 1.4 |
| | 30 | 0.1 | >99.0 | 0.1 | 3.2 |
| | 60 | 0.0 | >99.0 | 0.1 | 5.2 |
| | 120 | 0.0 | >99.0 | 0.1 | 7.5 |
| | 240 | 0.0 | >99.0 | 0.1 | 10.9 |
| 204 | 0 | 100.0 | 4.4 | 100.00 | 0.0 |
| | 10 | 38.7 | 48.5 | 97.40 | 0.2 |
| | 30 | 5.9 | 72.6 | 94.26 | 0.4 |
| | 60 | 0.6 | 70.2 | 92.22 | 0.6 |
| | 120 | 0.1 | 68.7 | 93.59 | 0.8 |
| | 240 | 0.0 | 65.6 | 91.87 | 1.2 |
| 205 | 0 | 100.0 | 33.0 | 100.0 | 0.4 |
| | 10 | 0.6 | >99.0 | 96.7 | 6.3 |
| | 30 | 0.1 | >99.0 | 90.7 | 13.0 |
| | 60 | 0.1 | >99.0 | 84.3 | 19.7 |
| | 120 | 0.1 | >99.0 | 80.3 | 26.8 |
| | 240 | 0.0 | >99.0 | 67.9 | 38.6 |
| 207 | 0 | 100.0 | 12.1 | 100.0 | 7.4 |
| | 10 | 26.5 | 90.7 | 42.0 | 69.3 |
| | 30 | 0.5 | >99.0 | 6.9 | 78.6 |
| | 60 | 0.0 | >99.0 | 1.6 | 81.9 |
| | 120 | 0.0 | >99.0 | 0.7 | 80.2 |
| | 240 | 0.0 | >99.0 | 0.1 | 76.4 |
| 208 | 0 | 100.0 | 79.0 | 100.0 | 76.3 |
| | 10 | 0.0 | >99.0 | 0.1 | 80.1 |
| | 30 | 0.0 | >99.0 | 0.2 | 76.9 |
| | 60 | 0.0 | >99.0 | 0.2 | 80.9 |
| | 120 | 0.0 | >99.0 | 0.0 | 77.5 |
| | 240 | 0.0 | >99.0 | 0.0 | 79.0 |
| 210 | 0 | 100.0 | >99.0 | 100.0 | 88.9 |
| | 10 | 0.0 | >99.0 | 0.5 | 90.6 |
| | 30 | 0.0 | >99.0 | 1.0 | 91.1 |
| | 60 | 0.0 | >99.0 | 0.6 | 91.4 |
| | 120 | 0.1 | >99.0 | 0.1 | 89.6 |
| | 240 | 0.1 | >99.0 | 0.1 | 88.7 |
| 209 | 0 | 100.0 | 4.7 | 100.0 | 0.2 |
| | 10 | 11.5 | 70.9 | 96.0 | 1.7 |
| | 30 | 0.0 | 79.5 | 91.8 | 3.4 |
| | 60 | 0.0 | 73.7 | 87.6 | 5.5 |
| | 120 | 0.0 | 71.1 | 84.1 | 9.3 |
| | 240 | 0.0 | 73.4 | 72.5 | 12.1 |
| 201 | 0 | 100.0 | 0.0 | 100.0 | 0.0 |
| | 10 | >99.0 | 0.0 | 96.3 | 0.0 |
| | 30 | >99.0 | 0.0 | 97.0 | 0.0 |
| | 60 | >99.0 | 0.0 | 94.8 | 0.0 |
| | 120 | >99.0 | 0.0 | 97.7 | 0.0 |
| | 240 | 97.6 | 0.0 | >99.0 | 0.0 |
| 206 | 0 | 100.0 | 18.3 | 100.0 | 0.1 |
| | 10 | 0.8 | 76.8 | >99.0 | 2.7 |
| | 30 | 0.1 | 80.0 | 92.7 | 5.1 |
| | 60 | 0.0 | 76.7 | 83.8 | 8.9 |
| | 120 | 0.0 | 73.3 | 79.1 | 13.9 |
| | 240 | 0.0 | 74.4 | 68.0 | 19.2 |

### Example B1: SERT Inhibition Assay

SERT inhibition was measured using a Neruotransmitter Transportation Fluorescence assay. Briefly, stable 5HTT HEK293 cells were prepared in a 384 microwell plate. Compounds were prepared by in assay buffer (20 mM HEPES in HBSS, 0.1% BSA) at a top concentration of 1 μM. 10 doses of test compound (3-fold serial dilution) were added to the plated cells and incubated for 30 minutes at 37° C. 25 μL of dye solution (Molecular Devices Neurotransmitter Transporter Uptake Assay Kit) was added per well and incubated for 30 minutes at 37° C. The plates were then read on a plate reader. The results are provided in Table 2 (n=6±SD).

TABLE 2

| Compound Name | Compound ID | SERT IC₅₀ (nM) |
|---|---|---|
| (−)-mesembranol | 018 | 21.9 ± 8.0 |
| (−)-6-epi-mesembranol | 019 | 10.6 ± 10.1 |

1-30. (canceled)

31. A compound of formula (I): 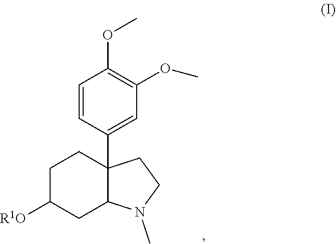
or a pharmaceutically acceptable salt thereof,
wherein:
R¹ is —C(O)NR²R³, —P(O)OR³OR⁴, —C(O)OR⁵, or —CH₂OC(O)R⁷;
R² is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein C₁-C₆ alkyl, C₂-C₆ alkenyl, C₃-C₁₀ cycloalkyl, phenyl, and 5- to 7-membered heteroaryl are optionally substituted with halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy;
R³ is H, C₁-C₆ alkyl, phenyl, —(CH₂O)ₙ—C(O)OC₁-C₆ alkyl, or —(CH₂O)ₙ—C(O)C₁-C₆ alkyl, wherein C₁-C₆ alkyl is optionally substituted with halo, hydroxy, C₁-C₃ alkyl, C₁-C₃ alkoxy, C₃-C₁₀ cycloalkyl, phenyl, 5- to 7-membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;
or R² and R³ taken together with the nitrogen atom to which they are attached form a 4- to 7-membered heterocycle, wherein the 4- to 7-membered heterocycle is optionally substituted with halo, hydroxy, C₁-C₃ alkyl, C₁-C₃ alkoxy, C₃-C₁₀ cycloalkyl, phenyl, 5- to 7-membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;
R⁴ is H, C₁-C₆ alkyl, phenyl, —(CH₂O)ₙ—C(O)OC₁-C₆ alkyl, or —(CH₂O)ₙ—C(O)C₁-C₆ alkyl, wherein C₁-C₆ alkyl is optionally substituted with halo, hydroxy, C₁-C₃ alkyl, C₁-C₃ alkoxy, C₃-C₁₀ cycloalkyl, phenyl, 5- to 7-membered heterocycle, 5- to 7-membered heteroaryl, nitro, —N (C₁-C₃ alkyl)₂, —NH₂, —N(H)C₁-C₃ alkyl, C₁-C₃ haloalkyl, —COOH, cyano, phenyl, or phenoxy;
or R³ and R⁴ taken together with the —O—P(O)—O— to which they are attached form a 5- to 7-membered heterocycle, wherein the 5- to 7-membered heterocycle is optionally substituted with halo, hydroxy, C₁-C₃ alkyl, C₁-C₃ alkoxy, C₃-C₁₀ cycloalkyl, phenyl, 5- to 7-membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy;
R⁵ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein C₁-C₆ alkyl, C₂-C₆ alkenyl, C₃-C₁₀ cycloalkyl, phenyl, and 5- to 7-membered heteroaryl are optionally substituted with halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy;
R⁷ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein C₁-C₆ alkyl, C₂-C₆ alkenyl, C₃-C₁₀ cycloalkyl, phenyl, and 5- to 7-membered heteroaryl are optionally substituted with halo, hydroxy, C₁-C₆ alkyl, C₁-C₃ alkoxy, nitro, —N(C₁-C₃ alkyl)₂, —NH₂, —N(H)C₁-C₃ alkyl, C₁-C₃ haloalkyl, —COOH, cyano, phenyl, or phenoxy; and
n is 1 or 2.

32. The compound of claim 31, wherein R¹ is —C(O)NR²R³.

33. The compound of claim 32, wherein R² and R³ taken together with the nitrogen atom to which they are attached form a 4- to 7-membered heterocycle, wherein the 4- to 7-membered heterocycle is optionally substituted with halo, hydroxy, C₁-C₃ alkyl, C₁-C₃ alkoxy, C₃-C₁₀ cycloalkyl, phenyl, 5- to 7-membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy.

34. The compound of claim 32, wherein R² is C₁-C₆ alkyl; and R³ is H, or C₁-C₆ alkyl.

35. The compound of claim 34, wherein the compound is of formula (II-A): 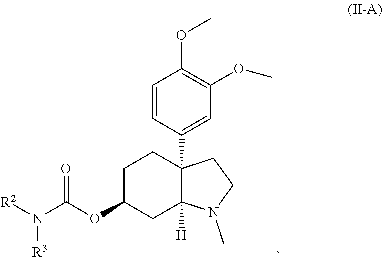
or a pharmaceutically acceptable salt thereof, wherein R² and R³ are independently H or methyl.

36. The compound of claim 34, wherein the compound is of formula (II-B): 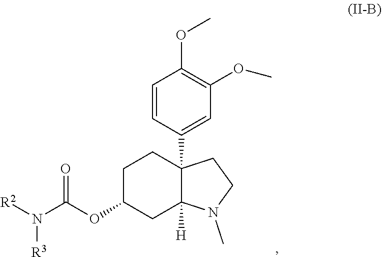
or a pharmaceutically acceptable salt thereof, wherein R² and R³ are independently H or methyl.

37. The compound of claim 34, wherein the compound is 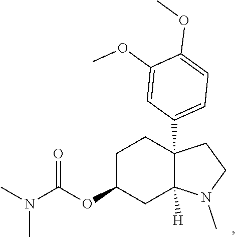 [SMILES: COc1ccc([C@@]23CC[C@H](OC(=O)N(C)C)C[C@@H]2N(C)CC3)cc1OC]
or a pharmaceutically acceptable salt thereof.

38. The compound of claim 31, wherein R¹ is —P(O)OR³OR⁴.

39. The compound of claim 38, wherein
a. R³ is H, C₁-C₆ alkyl, phenyl, —(CH₂O)ₙ—C(O)OC₁-C₆ alkyl, or —(CH₂O)ₙ—C(O)C₁-C₆ alkyl, wherein C₁-C₆ alkyl is optionally substituted with halo, hydroxy, C₁-C₃ alkyl, C₁-C₃ alkoxy, C₃-C₁₀ cycloalkyl, phenyl, 5- to 7-membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy; and
b. R⁴ is H, C₁-C₆ alkyl, phenyl, —(CH₂O)ₙ—C(O)OC₁-C₆ alkyl, or —(CH₂O)ₙ—C(O)C₁-C₆ alkyl, wherein C₁-C₆ alkyl is optionally substituted with halo, hydroxy, C₁-C₃ alkyl, C₁-C₃ alkoxy, C₃-C₁₀ cycloalkyl, phenyl, 5- to 7-membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy.

40. The compound of claim 39, wherein R³ is H or C₁-C₆ alkyl and R⁴ is H or C₁-C₆ alkyl.

41. The compound of claim 40, wherein the compound is 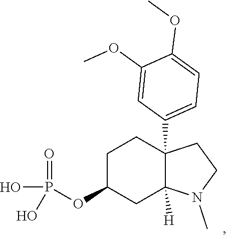 [SMILES: COc1ccc([C@@]23CC[C@H](OP(=O)(O)O)C[C@@H]2N(C)CC3)cc1OC]
or a pharmaceutically acceptable salt thereof.

42. The compound of claim 38, wherein R³ and R⁴ taken together with the —O—P(O)—O— to which they are attached form a 5- to 7-membered heterocycle, wherein the 5-7 membered heterocycle is optionally substituted with halo, hydroxy, C₁-C₃ alkyl, C₁-C₃ alkoxy, C₃-C₁₀ cycloalkyl, phenyl, 5- to 7-membered heterocycle, 5- to 7-membered heteroaryl, nitro, —N(C₁-C₃ alkyl)₂, —NH₂, —N(H)C₁-C₃ alkyl, C₁-C₃ haloalkyl, —COOH, cyano, phenyl, or phenoxy.

43. The compound of claim 42, wherein R³ and R⁴ taken together with the —O—P(O)—O— to which they are attached form a 6-membered heterocycle.

44. The compound of claim 31, wherein R¹ is —CH₂OC(O)R⁷.

45. The compound of claim 44, wherein R⁷ is C₁-C₆ alkyl.

46. The compound of claim 45, wherein the compound is 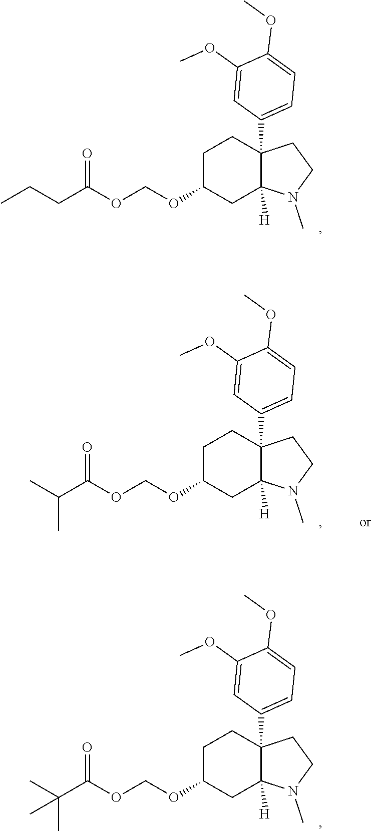 [SMILES: CCCC(=O)OCO[C@@H]1CC[C@@]2(c3ccc(OC)c(OC)c3)CCN(C)[C@H]2C1 ; COc1ccc([C@@]23CC[C@@H](OCOC(=O)C(C)C)C[C@@H]2N(C)CC3)cc1OC ; COc1ccc([C@@]23CC[C@@H](OCOC(=O)C(C)(C)C)C[C@@H]2N(C)CC3)cc1OC]
or a pharmaceutically acceptable salt thereof.

47. A compound of formula (I): 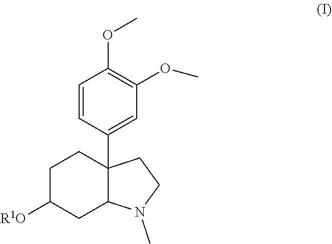
or a pharmaceutically acceptable salt thereof,
wherein:
R¹ is —C(O)R⁶; and
R⁶ is C₁-C₆ alkyl, C₂-C₆ alkenyl, C₃-C₁₀ cycloalkyl, C₁-C₃ alkyl-C₃-C₁₀ cycloalkyl, phenyl, or 5- to 7-membered heteroaryl, wherein C₁-C₆ alkyl, C₂-C₆ alkenyl, C₃-C₁₀ cycloalkyl, phenyl, and 5- to 7-membered heteroaryl are optionally substituted with halo, hydroxy, C₁-C₆ alkyl, C₁-C₃ alkoxy, —OC(O)C₁-C₆ alkyl, nitro, —N(C₁-C₃ alkyl)₂, —NH₂, —N(H)C₁-C₃ alkyl, C₁-C₃ haloalkyl, —COOH, cyano, phenyl, or phenoxy;
provided the compound is not

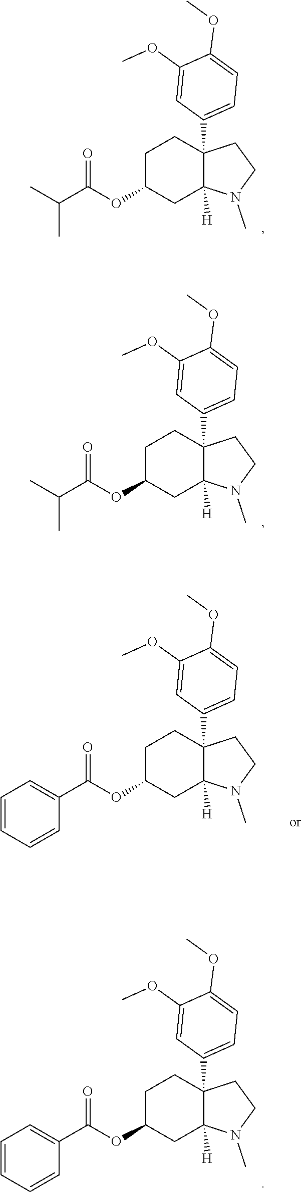 [SMILES: COc1ccc([C@@]23CC[C@@H](OC(=O)C(C)C)C[C@@H]2N(C)CC3)cc1OC ; COc1ccc([C@@]23CC[C@H](OC(=O)C(C)C)C[C@@H]2N(C)CC3)cc1OC ; COc1ccc([C@@]23CC[C@@H](OC(=O)c4ccccc4)C[C@@H]2N(C)CC3)cc1OC ; COc1ccc([C@@]23CC[C@H](OC(=O)c4ccccc4)C[C@@H]2N(C)CC3)cc1OC]

48. The compound of claim 47, wherein R⁶ is C₁-C₆ alkyl.

49. A pharmaceutical composition, comprising a compound of claim 31, and a pharmaceutically acceptable excipient.

50. A method of treating social anxiety disorder, generalized anxiety disorder or depression, the method comprising administering to a mammal in need thereof an effective amount of a compound of claim 31.
